(19) United States
(12) Patent Application Publication
(10) Pub. No.: US 2024/0355472 A1
(43) Pub. Date: Oct. 24, 2024

(54) Deep Learning and Artificial Intelligence-Based Non-Sequence Altering Change Latent Space using Variational Autoencoders (VAEs)

(71) Applicant: H42 Inc., Foster City, CA (US)

(72) Inventors: Wim VAN CRIEKINGE, Foster City, CA (US); An Nijs, Sint-Amandsber (BE); Johan Vandersmissen, Hoeselt (BE)

(73) Assignee: H42 Inc., Foster City, CA (US)

(21) Appl. No.: 18/604,287

(22) Filed: Mar. 13, 2024

Related U.S. Application Data
(60) Provisional application No. 63/527,675, filed on Jul. 19, 2023.
(60) Provisional application No. 63/527,674, filed on Jul. 19, 2023.
(60) Provisional application No. 63/451,916, filed on Mar. 13, 2023.

(51) Int. Cl. G16H 50/20; C12Q 1/6834; C12Q 1/686; G01N 21/64; G16B 40/30; G16H 50/30

(52) U.S. Cl. CPC G16H 50/20; C12Q 1/6834; C12Q 1/686; G01N 21/6486; G16B 40/30; G16H 50/30; C12Q 2600/154

(57) ABSTRACT

A variational autoencoder, comprising an encoder neural network and a decoder neural network, is configured to process a plurality of non-sequence altering change marker inputs, and to encode them in a non-sequence altering change latent space, which comprises respective feature embeddings that compress respective non-sequence altering change marker inputs in the plurality of non-sequence altering change marker inputs into a jointly Gaussian distribution. A method for predictive diagnosis of rheumatoid arthritis (RA) in a patient is also disclosed. Selected marker genes associated with patients having rheumatoid arthritis are formed by methods including deep learning analysis (e.g., via the variational autoencoder). A patient's blood products are assayed to detect the marker genes associated with rheumatoid arthritis. Selected marker genes include RCNA3, HDAC4, and SIPA1. The method may be extended to detect diseases different than or in addition to rheumatoid arthritis.

### PRIORITY APPLICATIONS

This application claims the benefit of and priority to U.S. Provisional Patent Application No. 63/451,916, titled “DEEP LEARNING FOR BIOLOGICAL REGULATION OF AGEING,” filed Mar. 13, 2023 (Attorney Docket No. H421003USP01). The priority application is incorporated herein by reference in its entirety and for all purposes as if completely and fully set forth herein.

This application claims the benefit of and priority to U.S. Provisional Patent Application No. 63/527,674, titled “VARIATIONAL AUTOENCODERS TO PREDICT DNA-METHYLATION AGE AND UNRAVEL BIOLOGICAL REGULATION OF AGEING,” filed Jul. 19, 2023 (Attorney Docket No. H421004USP01). The priority application is incorporated herein by reference in its entirety and for all purposes as if completely and fully set forth herein.

This application claims the benefit of and priority to U.S. Provisional Patent Application No. 63/527,675, titled “METHYLATION DIFFERENCES IN RHEUMATOID ARTHRITIS PATIENTS COMPARED TO NORMAL CONTROLS USING MACHINE LEARNING,” filed Jul. 19, 2023 (Attorney Docket No. H421005USP01). The priority application is incorporated herein by reference in its entirety and for all purposes as if completely and fully set forth herein.

### INCORPORATIONS BY REFERENCE

The following are hereby incorporated by reference in their entirety and for all purposes as if completely and fully set forth herein:
U.S. Nonprovisional patent application Ser. No. 17/525,552, filed Nov. 12, 2021, titled “EPIGENETIC AGE PREDICTOR”;
U.S. Nonprovisional patent application Ser. No. 17/831,427, now U.S. Pat. No. 11,781,175 B1, filed Jun. 2, 2022, titled “PCR-BASED EPIGENETIC AGE PREDICTION”;
U.S. Nonprovisional patent application Ser. No. 17/831,430, filed Jun. 2, 2022, titled “EPIGENETIC AGE PREDICTOR”; and
PCT Application No. PCT/IB2022/060944, filed Nov. 14, 2022, titled “GENERATION OF EPIGENETIC AGE INFORMATION”.

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Deep learning is a frontier for artificial intelligence, aiming to be closer to its primary goal—artificial intelligence. Deep learning has seen great success in a wide variety of applications, such as natural language processing, speech recognition, medical applications, computer vision, and intelligent transportation systems. The great success of deep learning is due to the larger models. The scale of these models has included hundreds of millions of parameters. These hundreds of millions of parameters allow the model to have more degrees of freedom enough to produce awe-inspiring description capability.

However, the large number of parameters requires a massive amount of training data with labels. Improving model performance by data annotation has two crucial challenges. On the one hand, the data growth rate is far behind the growth rate of model parameters, so data growth has primarily hindered the further development of the model. On the other hand, the emergence of new tasks has far exceeded the speed of data updates, and annotating for all samples is laborious.

To tackle this challenge, new datasets are built by generating synthetic samples, thereby speeding up model iteration and reducing the cost of data annotation. Pre-training methods and transfer learning have also been used to solve this challenge, such as Transformers, BERT, and GPT. These works have achieved incredible results.

However, the generated data is only used as base data to initialize the model. In order to obtain a high-precision usable model, it is often necessary to label and update specific data.

Integrating apriori knowledge in the learning framework is an effective means to deal with sparse data, as the learner does not need to induce the knowledge from the data itself. As special agents, humans have rich prior knowledge. If the machine can learn human wisdom and knowledge, it will help deal with sparse data.

Human-in-the-loop (HITL) addresses these issues by incorporating human knowledge into the modeling process. HITL aims to train an accurate prediction model with minimum cost by integrating human knowledge and experience. Humans can provide training data for machine learning applications and directly accomplish some tasks that are hard for computers in the pipeline with the help of machine-based approaches.

At present, there is still a high degree of coupling between deep learning tasks and data, and the performance of deep learning largely depends on the quality of the data. For a new task, if you want to obtain better performance, you need to provide a large amount of high-quality labeled data. However, the labeled data requires a large amount of labor. In addition, large-scale data annotation takes a long time, and many iterations of tasks cannot wait such a long time. Unlike weak annotate and automatic annotate, HITL-based methods emphasize finding the key samples that play a decisive factor in new sample data.

A core set is a weighted subset of a larger set. A core set guarantees that a model fitting the core set also fits the larger set. Core set construction methods perform importance sampling with respect to sensitivity score, to provide high-probability solutions for a particular problem, such as k-means and k-median clustering, naïve Bayes and nearest-neighbors, mixture models, low rank approximation, spectral approximation, Nystrom methods, and Bayesian inference.

Supervised learning usually requires a large set of labeled data to train the prediction model. As the learning algorithms become more and more complicated, the required size of training set gets larger and larger. Meanwhile, labeling data examples is rather expensive, because the annotation process is usually time-consuming and needs high expertise in some difficult tasks. It is thus a significant challenge to learn with insufficient labeled data.

Active learning is a primary approach to overcome this challenge. It iteratively selects the most useful examples from the unlabeled dataset to query their labels from the oracle. After adding the newly labeled data into the training set, the model can be updated to achieve better performance. The key task in active learning is how to accurately estimate the potential utility of an example on improving the performance, such that the model can be well trained with minimal queries.

An opportunity arises to use human-in-the-loop (HITL) active learning for core set discovery. Accelerated deep learning with minimal coding may result.

Epigenetic ageing clocks based on age-associated changes in DNA-methylation (DNAm) profiles have shown great potential as predictors to quantify age-related health and disease. The majority of these clocks are built using penalized linear regression models with the aim to predict chronological and/or biological age. However, the precise underlying biological mechanisms influencing these clocks remain unclear. In this work, we explored if a Variational Autoencoder (VAE) can be trained to model DNAm age, and whether this VAE captures biologically relevant features of ageing in the latent space.

Epigenetic ageing clocks based on age-associated changes in DNA-methylation (DNAm) profiles have shown great potential as predictors to quantify age-related health and disease. The majority of these clocks are built using penalized linear regression models with the aim to predict chronological and/or biological age. However, the precise underlying biological mechanisms influencing these clocks remain unclear. In this work, we explored if a Variational Autoencoder (VAE) can be trained to model DNAm age, and whether this VAE captures biologically relevant features of ageing in the latent space.

Genomics, in the broad sense, also referred to as functional genomics, aims to characterize the function of every genomic element of an organism by using genome-scale assays such as genome sequencing, transcriptome profiling and proteomics. Genomics arose as a data driven science, and operates by discovering novel properties from explorations of genome-scale data rather than by testing preconceived models and hypotheses. Applications of genomics include finding associations between genotype and phenotype, discovering biomarkers for patient stratification, predicting the function of genes, and charting biochemically active genomic regions such as transcriptional enhancers.

Genomics data are too large and too complex to be mined solely by visual investigation of pairwise correlations. Instead, analytical tools are required to support the discovery of unanticipated relationships, to derive novel hypotheses and models and to make predictions. Unlike some algorithms, in which assumptions and domain expertise are hard coded, machine learning algorithms are designed to automatically detect patterns in data. Hence, machine learning algorithms are suited to data-driven sciences and, in particular, to genomics. However, the performance of machine learning algorithms can strongly depend on how the data are represented, that is, on how each variable (also called a feature) is computed. For instance, to classify a tumor as malign or benign from a fluorescent microscopy image, a preprocessing algorithm could detect cells, identify the cell type, and generate a list of cell counts for each cell type.

A machine learning model can take the estimated cell counts, which are examples of handcrafted features, as input features to classify the tumor. A central issue is that classification performance depends heavily on the quality and the relevance of these features. For example, relevant visual features such as cell morphology, distances between cells or localization within an organ are not captured in cell counts, and this incomplete representation of the data may reduce classification accuracy.

Deep learning, a subdiscipline of machine learning, addresses this issue by embedding the computation of features into the machine learning model itself to yield end-to-end models. This outcome has been realized through the development of deep neural networks, machine learning models that comprise successive elementary operations, which compute increasingly more complex features by taking the results of preceding operations as input. Deep neural networks are able to improve prediction accuracy by discovering relevant features of high complexity, such as the cell morphology and spatial organization of cells in the above example. The construction and training of deep neural networks have been enabled by the explosion of data, algorithmic advances, and substantial increases in computational capacity, particularly through the use of graphical processing units (GPUs).

The goal of supervised learning is to obtain a model that takes features as input and returns a prediction for a so-called target variable. An example of a supervised learning problem is one that predicts whether an intron is spliced out or not (the target) given features on the RNA such as the presence or absence of the canonical splice site sequence, the location of the splicing branchpoint or intron length. Training a machine learning model refers to learning its parameters, which commonly involves minimizing a loss function on training data with the aim of making accurate predictions on unseen data.

For many supervised learning problems in computational biology, the input data can be represented as a table with multiple columns, or features, each of which contains numerical or categorical data that are potentially useful for making predictions. Some input data are naturally represented as features in a table (such as temperature or time), whereas other input data need to be first transformed (such as deoxyribonucleic acid (DNA) sequence into k-mer counts) using a process called feature extraction to fit a tabular representation. For the intron-splicing prediction problem, the presence or absence of the canonical splice site sequence, the location of the splicing branchpoint and the intron length can be preprocessed features collected in a tabular format. Tabular data are standard for a wide range of supervised machine learning models, ranging from simple linear models, such as logistic regression, to more flexible nonlinear models, such as neural networks and many others.

Logistic regression is a binary classifier, that is, a supervised learning model that predicts a binary target variable. Specifically, logistic regression predicts the probability of the positive class by computing a weighted sum of the input features mapped to the [0, 1] interval using the sigmoid function, a type of activation function. The parameters of logistic regression, or other linear classifiers that use different activation functions, are the weights in the weighted sum. Linear classifiers fail when the classes, for instance, that of an intron spliced out or not, cannot be well discriminated with a weighted sum of input features. To improve predictive performance, new input features can be manually added by transforming or combining existing features in new ways, for example, by taking powers or pairwise products.

Neural networks use hidden layers to learn these nonlinear feature transformations automatically. Each hidden layer can be thought of as multiple linear models with their output transformed by a nonlinear activation function, such as the sigmoid function or the more popular rectified-linear unit (ReLU). Together, these layers compose the input features into relevant complex patterns, which facilitates the task of distinguishing two classes.

Deep neural networks use many hidden layers, and a layer is said to be fully-connected when each neuron receives inputs from all neurons of the preceding layer. Neural networks are commonly trained using stochastic gradient descent, an algorithm suited to training models on very large data sets. Implementation of neural networks using modern deep learning frameworks enables rapid prototyping with different architectures and data sets. Fully-connected neural networks can be used for a number of genomics applications, which include predicting the percentage of exons spliced in for a given sequence from sequence features such as the presence of binding motifs of splice factors or sequence conservation; prioritizing potential disease-causing genetic variants; and predicting cis-regulatory elements in a given genomic region using features such as chromatin marks, gene expression and evolutionary conservation.

Local dependencies in spatial and longitudinal data must be considered for effective predictions. For example, shuffling a DNA sequence or the pixels of an image severely disrupts informative patterns. These local dependencies set spatial or longitudinal data apart from tabular data, for which the ordering of the features is arbitrary. Consider the problem of classifying genomic regions as bound versus unbound by a particular transcription factor, in which bound regions are defined as high-confidence binding events in chromatin immunoprecipitation following by sequencing (ChlP-seq) data. Transcription factors bind to DNA by recognizing sequence motifs. A fully-connected layer based on sequence-derived features, such as the number of k-mer instances or the position weight matrix (PWM) matches in the sequence, can be used for this task. As k-mer or PWM instance frequencies are robust to shifting motifs within the sequence, such models could generalize well to sequences with the same motifs located at different positions. However, they would fail to recognize patterns in which transcription factor binding depends on a combination of multiple motifs with well-defined spacing. Furthermore, the number of possible kmers increases exponentially with k-mer length, which poses both storage and overfitting challenges.

A convolutional layer is a special form of fully-connected layer in which the same fully-connected layer is applied locally, for example, in a 6 bp window, to all sequence positions. This approach can also be viewed as scanning the sequence using multiple PWMs, for example, for transcription factors GATAI and TAL I. By using the same model parameters across positions, the total number of parameters is drastically reduced, and the network is able to detect a motif at positions not seen during training. Each convolutional layer scans the sequence with several filters by producing a scalar value at every position, which quantifies the match between the filter and the sequence. As in fully-connected neural networks, a nonlinear activation function (commonly ReLU) is applied at each layer. Next, a pooling operation is applied, which aggregates the activations in contiguous bins across the positional axis, commonly taking the maximal or average activation for each channel. Pooling reduces the effective sequence length and coarsens the signal. The subsequent convolutional layer composes the output of the previous layer and is able to detect whether a GATAI motif and TALI motif were present at some distance range. Finally, the output of the convolutional layers can be used as input to a fully-connected neural network to perform the final prediction task. Hence, different types of neural network layers (e.g., fully-connected layers and convolutional layers) can be combined within a single neural network.

Convolutional neural networks (CNNs) can predict various molecular phenotypes on the basis of DNA sequence alone. Applications include classifying transcription factor binding sites and predicting molecular phenotypes such as chromatin features, DNA contact maps, DNA methylation, gene expression, translation efficiency, RBP binding, and microRNA (miRNA) targets. In addition to predicting molecular phenotypes from the sequence, convolutional neural networks can be applied to more technical tasks traditionally addressed by handcrafted bioinformatics pipelines. For example, convolutional neural networks can predict the specificity of guide RNA, denoise ChlP-seq, enhance Hi-C data resolution, predict the laboratory of origin from DNA sequences and call genetic variants. Convolutional neural networks have also been employed to model long-range dependencies in the genome. Although interacting regulatory elements may be distantly located on the unfolded linear DNA sequence, these elements are often proximal in the actual 3D chromatin conformation. Hence, modelling molecular phenotypes from the linear DNA sequence, albeit a crude approximation of the chromatin, can be improved by allowing for long-range dependencies and allowing the model to implicitly learn aspects of the 3D organization, such as promoter-enhancer looping. This is achieved by using dilated convolutions, which have a receptive field of up to 32 kb. Dilated convolutions also allow splice sites to be predicted from sequence using a receptive field of 10 kb, thereby enabling the integration of genetic sequence across distances as long as typical human introns (See Jaganathan, K. et al. Predicting splicing from primary sequence with deep learning. Cell 176, 535-548 (2019)).

Different types of neural network can be characterized by their parameter-sharing schemes. For example, fully-connected layers have no parameter sharing, whereas convolutional layers impose translational invariance by applying the same filters at every position of their input. Recurrent neural networks (RNNs) are an alternative to convolutional neural networks for processing sequential data, such as DNA sequences or time series, that implement a different parameter-sharing scheme. Recurrent neural networks apply the same operation to each sequence element. The operation takes as input the memory of the previous sequence element and the new input. It updates the memory and optionally emits an output, which is either passed on to subsequent layers or is directly used as model predictions. By applying the same model at each sequence element, recurrent neural networks are invariant to the position index in the processed sequence. For example, a recurrent neural network can detect an open reading frame in a DNA sequence regardless of the position in the sequence. This task requires the recognition of a certain series of inputs, such as the start codon followed by an in-frame stop codon.

The main advantage of recurrent neural networks over convolutional neural networks is that they are, in theory, able to carry over information through infinitely long sequences via memory. Furthermore, recurrent neural networks can naturally process sequences of widely varying length, such as mRNA sequences. However, convolutional neural networks combined with various tricks (such as dilated convolutions) can reach comparable or even better performances than recurrent neural networks on sequence-modelling tasks, such as audio synthesis and machine translation. Recurrent neural networks can aggregate the outputs of convolutional neural networks for predicting single-cell DNA methylation states, RBP binding, transcription factor binding, and DNA accessibility. Moreover, because recurrent neural networks apply a sequential operation, they cannot be easily parallelized and are hence much slower to compute than convolutional neural networks.

Each human has a unique genetic code, though a large portion of the human genetic code is common for all humans. In some cases, a human genetic code may include an outlier, called a genetic variant, that may be common among individuals of a relatively small group of the human population. For example, a particular human protein may comprise a specific sequence of amino acids, whereas a variant of that protein may differ by one amino acid in the otherwise same specific sequence.

Genetic variants may be pathogenetic, leading to diseases. Though most of such genetic variants have been depleted from genomes by natural selection, an ability to identify which genetic variants are likely to be pathogenic can help researchers focus on these genetic variants to gain an understanding of the corresponding diseases and their diagnostics, treatments, or cures. The clinical interpretation of millions of human genetic variants remains unclear. Some of the most frequent pathogenic variants are single nucleotide missense mutations that change the amino acid of a protein. However, not all missense mutations are pathogenic.

Models that can predict molecular phenotypes directly from biological sequences can be used as in silico perturbation tools to probe the associations between genetic variation and phenotypic variation and have emerged as new methods for quantitative trait loci identification and variant prioritization. These approaches are of major importance given that the majority of variants identified by genome-wide association studies of complex phenotypes are non-coding, which makes it challenging to estimate their effects and contribution to phenotypes. Moreover, linkage disequilibrium results in blocks of variants being co-inherited, which creates difficulties in pinpointing individual causal variants. Thus, sequence-based deep learning models that can be used as interrogation tools for assessing the impact of such variants offer a promising approach to find potential drivers of complex phenotypes. One example includes predicting the effect of noncoding single-nucleotide variants and short insertions or deletions (indels) indirectly from the difference between two variants in terms of transcription factor binding, chromatin accessibility or gene expression predictions. Another example includes predicting novel splice site creation from sequence or quantitative effects of genetic variants on splicing.

Human health and age can be measured in a variety of different ways. A human's chronological age, that is a time that an individual is alive, is one form of measure of a human's health and age. Another form of measuring a human's age is a subjective biological age that is used to account for a shortfall between a population average life expectancy and the perceived life expectancy of an individual of the same age. A human's environment or behavior can cause their body to biologically age at an accelerated rate. It has been difficult in the past to estimate an individual's biological age with accuracy.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which.

FIG. 1 illustrates one implementation of a system-level diagram of the technology disclosed.

FIG. 2 is a flowchart showing one method implementation of the technology disclosed.

FIG. 3 shows one implementation of a model Architecture of the disclosed variational autoencoder (VAE) to embed the methylation data into a latent space.

FIG. 4 shows one implementation of a three-layer multi-layer perceptron (MLP) for age prediction using the VAE embedding as input features.

FIG. 5 illustrates one implementation of the disclosed logistic regression model for binary classification to predict rheumatoid arthritis (RA) status from the VAE embedding.

FIGS. 6A and 6B illustrate another implementation of a system-level diagram of the technology disclosed.

FIG. 7 depict one implementation of the disclosed imAGE construction.

FIG. 8 is a flowchart showing another method implementation of the technology disclosed.

FIG. 9 shows another implementation of a model Architecture of the disclosed variational autoencoder (VAE) to embed the methylation data into a latent space.

FIG. 10 shows another implementation of a three-layer multi-layer perceptron (MLP) for age prediction using the VAE embedding as input features.

FIG. 11 illustrates another implementation of the disclosed logistic regression model for binary classification to predict rheumatoid arthritis (RA) status from the VAE embedding.

FIG. 12 shows different logics and functions of the technology disclosed.

FIG. 13 shows the original age density distribution (panel A) and the resulting density distribution of one such bootstrap (panel B).

FIGS. 14, 15, 16, 17, 18, and 19 shows various performance results of the technology disclosed.

FIG. 20 illustrates a diagram showing an example portion of DNA.

FIG. 21 illustrates a molecular structure diagram showing an example methylated cytosine molecule.

FIG. 22 illustrates a diagram showing an example portion of DNA.

FIG. 23A-N illustrate diagrams showing genomic diagrams of chromosomes.

FIG. 24 illustrates a diagram showing an example array chip used to detect methylation.

FIG. 25 illustrates a diagram showing example beads that respond to methylated and unmethylated CpG sites.

FIG. 26 illustrates a flow diagram showing an example operation of training a model.

FIGS. 27-28 illustrate flow diagrams showing example operations of predicting an epigenetic age.

FIG. 29 illustrates a flow diagram showing an example operation of processing a sample to provide an epigenetic age to a user.

FIGS. 30A-30H illustrate diagrams showing example amplifications of portions of DNA.

FIG. 31 illustrates a flow diagram showing an example operation of acquiring a sample from a user.

FIG. 32 shows an example computer system that can be used to implement the technology disclosed.

FIG. 33 is a schematic representation of an encoder-decoder architecture.

FIG. 34 shows an overview of an attention mechanism added onto an RNN encoder-decoder architecture.

FIG. 35 is a schematic representation of the calculation of self-attention showing one attention head.

FIG. 36 is a depiction of several attention heads in a Transformer block.

FIG. 37 is an illustration that shows how one can use multiple workers to compute the multi-head attention in parallel, as the respective heads compute their outputs independently of one another.

FIG. 38 is a portrayal of one encoder layer of a Transformer network.

FIG. 39 shows a schematic overview of a Transformer model.

FIGS. 40A and 40B is a depiction of a Vision Transformer (ViT).

FIGS. 41A, 41B, 41C and 41D illustrate a processing flow of the Vision Transformer (ViT).

FIG. 42 shows example software code that implements a Transformer block.

FIGS. 43A and 43B are flowcharts of one implementation of the technology disclosed forming marker genes for predicting the likelihood of a patient contracting rheumatoid arthritis.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The detailed description of various implementations will be better understood when read in conjunction with the appended drawings. To the extent that the figures illustrate diagrams of the functional blocks of the various implementations, the functional blocks are not necessarily indicative of the division between hardware circuitry. Thus, for example, one or more of the functional blocks (e.g., modules, processors, or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various implementations are not limited to the arrangements and instrumentality shown in the drawings.

The processing engines and databases of the figures, designated as modules, can be implemented in hardware or software, and need not be divided up in precisely the same blocks as shown in the figures. Some of the modules can also be implemented on different processors, computers, or servers, or spread among a number of different processors, computers, or servers. In addition, it will be appreciated that some of the modules can be combined, operated in parallel or in a different sequence than that shown in the figures without affecting the functions achieved. The modules in the figures can also be thought of as flowchart steps in a method. A module also need not necessarily have all its code disposed contiguously in memory; some parts of the code can be separated from other parts of the code with code from other modules or other functions disposed in between. Wherever practicable, similar or like reference characters are used in the drawings to indicate similar or like functionality.

### INTRODUCTION

Some implementations of the technology disclosed relate to using a Transformer model to provide an AI system. In particular, the technology disclosed proposes a parallel input, parallel output (PIPO) AI system based on the Transformer architecture. The Transformer model relies on a self-attention mechanism to compute a series of context-informed vector-space representations of elements in the input sequence and the output sequence, which are then used to predict distributions over subsequent elements as the model predicts the output sequence element-by-element. Not only is this mechanism straightforward to parallelize, but as each input's representation is also directly informed by all other inputs' representations, this results in an effectively global receptive field across the whole input sequence. This stands in contrast to, e.g., convolutional architectures which typically only have a limited receptive field.

In one implementation, the disclosed AI system is a multilayer perceptron (MLP). In another implementation, the disclosed AI system is a feedforward neural network. In yet another implementation, the disclosed AI system is a fully connected neural network. In a further implementation, the disclosed AI system is a fully convolution neural network. In a yet further implementation, the disclosed AI system is a semantic segmentation neural network. In a yet another further implementation, the disclosed AI system is a generative adversarial network (GAN) (e.g., CycleGAN, StyleGAN, pixelRNN, text-2-image, DiscoGAN, IsGAN). In a yet another implementation, the disclosed AI system includes self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, various ChatGPT versions, various LLaMA versions, BERT, SpanBERT, RoBERTa, XLNet, ELECTRA, UniLM, BART, T5, ERNIE (THU), KnowBERT, DeiT-Ti, DeiT-S, DeiT-B, T2T-ViT-14, T2T-ViT-19, T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCiT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN 41 FPN, DETR-DC5, TSP-FCOS, TSP-RCNN, ACT41MKDD (L=32), ACT41MKDD (L=16), SMCA, Efficient DETR, UP-DETR, UP-DETR, ViTB/16-FRCNN, ViT-B/16-FRCNN, PVT-Small41RetinaNet, Swin-T41RetinaNet, Swin-T4IATSS, PVT-Small41DETR, TNT-S41DETR, YOLOS-Ti, YOLOS-S, and YOLOS-B.

In one implementation, the disclosed AI system is a convolution neural network (CNN) with a plurality of convolution layers. In another implementation, the disclosed AI system is a recurrent neural network (RNN) such as a long short-term memory network (LSTM), bi-directional LSTM (Bi-LSTM), or a gated recurrent unit (GRU). In yet another implementation, the disclosed AI system includes both a CNN and an RNN.

In yet other implementations, the disclosed AI system can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The disclosed AI system can use one or more loss functions such as logistic regression/log loss, multi-class cross-entropy/softmax loss, binary cross-entropy loss, mean-squared error loss, L1 loss, L2 loss, smooth L1 loss, and Huber loss. The disclosed AI system can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The disclosed AI system can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms.

The disclosed AI system can be a linear regression model, a logistic regression model, an Elastic Net model, a support vector machine (SVM), a random forest (RF), a decision tree, and a boosted decision tree (e.g., XGBoost), or some other tree-based logic (e.g., metric trees, kd-trees, R-trees, universal B-trees, X-trees, ball trees, locality sensitive hashes, and inverted indexes). The disclosed AI system can be an ensemble of multiple models, in some implementations.

In some implementations, the disclosed AI system can be trained using backpropagation-based gradient update techniques. Example gradient descent techniques that can be used for training the disclosed AI system include stochastic gradient descent, batch gradient descent, and mini-batch gradient descent. Some examples of gradient descent optimization algorithms that can be used to train the disclosed AI system are Momentum, Nesterov accelerated gradient, Adagrad, Adadelta, RMSprop, Adam, AdaMax, Nadam, and AMSGrad.

### Transformer Logic

Machine learning is the use and development of computer systems that can learn and adapt without following explicit instructions, by using algorithms and statistical models to analyze and draw inferences from patterns in data. Some of the state-of-the-art models use Transformers, a more powerful and faster model than neural networks alone. Transformers originate from the field of natural language processing (NLP), but can be used in computer vision and many other fields. Neural networks process input in series and weight relationships by distance in the series. Transformers can process input in parallel and do not necessarily weigh by distance. For example, in natural language processing, neural networks process a sentence from beginning to end with the weights of words close to each other being higher than those further apart. This leaves the end of the sentence very disconnected from the beginning causing an effect called the vanishing gradient problem. Transformers look at each word in parallel and determine weights for the relationships to each of the other words in the sentence. These relationships are called hidden states because they are later condensed for use into one vector called the context vector. Transformers can be used in addition to neural networks. This architecture is described here.

### Encoder-Decoder Architecture

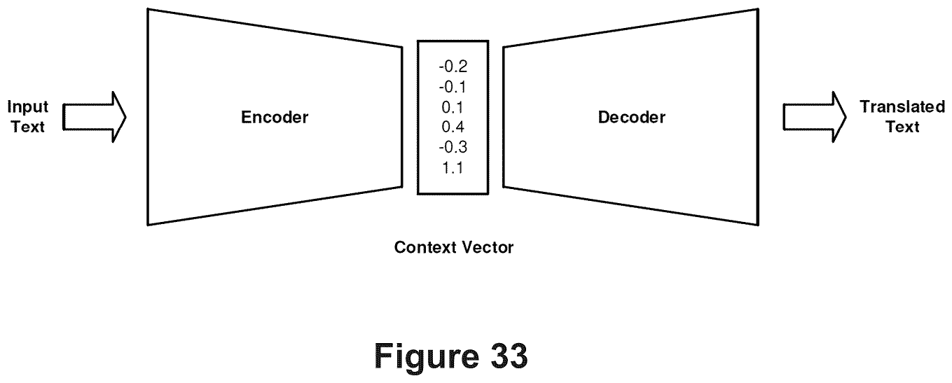

FIG. 33 is a schematic representation of an encoder-decoder architecture. This architecture is often used for NLP and has two main building blocks. The first building block is the encoder that encodes an input into a fixed-size vector. In the system we describe here, the encoder is based on a recurrent neural network (RNN). At each time step, t, a hidden state of time step, t−1, is combined with the input value at time step t to compute the hidden state at timestep t. The hidden state at the last time step, encoded in a context vector, contains relationships encoded at all previous time steps. For NLP, each step corresponds to a word. Then the context vector contains information about the grammar and the sentence structure. The context vector can be considered a low-dimensional representation of the entire input space. For NLP, the input space is a sentence, and a training set consists of many sentences.

The context vector is then passed to the second building block, the decoder. For translation, the decoder has been trained on a second language. Conditioned on the input context vector, the decoder generates an output sequence. At each time step, t, the decoder is fed the hidden state of time step, t−1, and the output generated at time step, t−1. The first hidden state in the decoder is the context vector, generated by the encoder. The context vector is used by the decoder to perform the translation.

The whole model is optimized end-to-end by using backpropagation, a method of training a neural network in which the initial system output is compared to the desired output and the system is adjusted until the difference is minimized. In backpropagation, the encoder is trained to extract the right information from the input sequence, the decoder is trained to capture the grammar and vocabulary of the output language. This results in a fluent model that uses context and generalizes well. When training an encoder-decoder model, the real output sequence is used to train the model to prevent mistakes from stacking. When testing the model, the previously predicted output value is used to predict the next one.

When performing a translation task using the encoder-decoder architecture, all information about the input sequence is forced into one vector, the context vector. Information connecting the beginning of the sentence with the end is lost, the vanishing gradient problem. Also, different parts of the input sequence are important for different parts of the output sequence, information that cannot be learned using only RNNs in an encoder-decoder architecture.

### Attention Mechanism

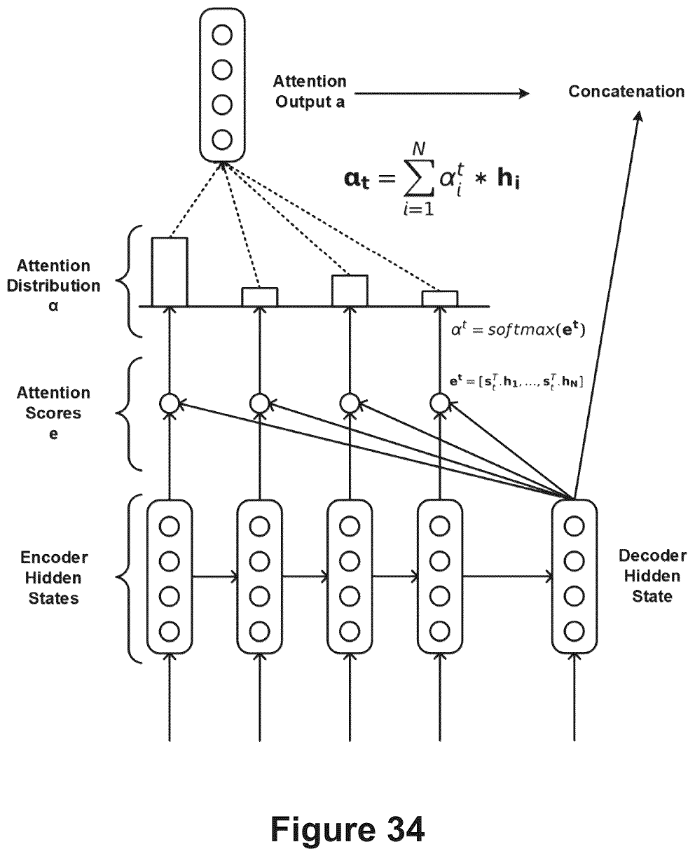

Attention mechanisms distinguish Transformers from other machine learning models. The attention mechanism provides a solution for the vanishing gradient problem. FIG. 34 shows an overview of an attention mechanism added onto an RNN encoder-decoder architecture. At every step, the decoder is given an attention score, e, for each encoder hidden state. In other words, the decoder is given weights for each relationship between words in a sentence. The decoder uses the attention score concatenated with the context vector during decoding. The output of the decoder at time step t is based on all encoder hidden states and the attention outputs. The attention output captures the relevant context for time step t from the original sentence. Thus, words at the end of a sentence may now have a strong relationship with words at the beginning of the sentence. In the sentence “The quick brown fox, upon arriving at the doghouse, jumped over the lazy dog,” fox and dog can be closely related despite being far apart in this complex sentence.

To weight encoder hidden states, a dot product between the decoder hidden state of the current time step, and all encoder hidden states, is calculated. This results in an attention score for every encoder hidden state. The attention scores are higher for those encoder hidden states that are similar to the decoder hidden state of the current time step. Higher values for the dot product indicate the vectors are pointing more closely in the same direction. The attention scores are converted to fractions that sum to one using the SoftMax function.

The SoftMax scores provide an attention distribution. The x-axis of the distribution is position in a sentence. The y-axis is attention weight. The scores show which encoder hidden states are most closely related. The SoftMax scores specify which encoder hidden states are the most relevant for the decoder hidden state of the current time step.

The elements of the attention distribution are used as weights to calculate a weighted sum over the different encoder hidden states. The outcome of the weighted sum is called the attention output. The attention output is used to predict the output, often in combination (concatenation) with the decoder hidden states. Thus, both information about the inputs, as well as the already generated outputs, can be used to predict the next outputs.

By making it possible to focus on specific parts of the input in every decoder step, the attention mechanism solves the vanishing gradient problem. By using attention, information flows more directly to the decoder. It does not pass through many hidden states. Interpreting the attention step can give insights into the data. Attention can be thought of as a soft alignment. The words in the input sequence with a high attention score align with the current target word. Attention describes long-range dependencies better than RNN alone. This enables analysis of longer, more complex sentences.

The attention mechanism can be generalized as: given a set of vector values and a vector query, attention is a technique to compute a weighted sum of the vector values, dependent on the vector query. The vector values are the encoder hidden states, and the vector query is the decoder hidden state at the current time step.

The weighted sum can be considered a selective summary of the information present in the vector values. The vector query determines on which of the vector values to focus. Thus, a fixed-size representation of the vector values can be created, in dependence upon the vector query.

The attention scores can be calculated by the dot product, or by weighing the different values (multiplicative attention).

### Embeddings

For most machine learning models, the input to the model needs to be numerical. The input to a translation model is a sentence, and words are not numerical. multiple methods exist for the conversion of words into numerical vectors. These numerical vectors are called the embeddings of the words. Embeddings can be used to convert any type of symbolic representation into a numerical one.

Embeddings can be created by using one-hot encoding. The one-hot vector representing the symbols has the same length as the total number of possible different symbols. Each position in the one-hot vector corresponds to a specific symbol. For example, when converting colors to a numerical vector, the length of the one-hot vector would be the total number of different colors present in the dataset. For each input, the location corresponding to the color of that value is one, whereas all the other locations are valued at zero. This works well for working with images. For NLP, this becomes problematic, because the number of words in a language is very large. This results in enormous models and the need for a lot of computational power. Furthermore, no specific information is captured with one-hot encoding. From the numerical representation, it is not clear that orange and red are more similar than orange and green. For this reason, other methods exist.

A second way of creating embeddings is by creating feature vectors. Every symbol has its specific vector representation, based on features. With colors, a vector of three elements could be used, where the elements represent the amount of yellow, red, and/or blue needed to create the color. Thus, all colors can be represented by only using a vector of three elements. Also, similar colors have similar representation vectors.

For NLP, embeddings based on context, as opposed to words, are small and can be trained. The reasoning behind this concept is that words with similar meanings occur in similar contexts. Different methods take the context of words into account. Some methods, like GloVe, base their context embedding on co-occurrence statistics from corpora (large texts) such as Wikipedia. Words with similar co-occurrence statistics have similar word embeddings. Other methods use neural networks to train the embeddings. For example, they train their embeddings to predict the word based on the context (Common Bag of Words), and/or to predict the context based on the word (Skip-Gram). Training these contextual embeddings is time intensive. For this reason, pre-trained libraries exist. Other deep learning methods can be used to create embeddings. For example, the latent space of a variational autoencoder (VAE) can be used as the embedding of the input. Another method is to use 1D convolutions to create embeddings. This causes a sparse, high-dimensional input space to be converted to a denser, low-dimensional feature space.

### Self-Attention: Queries (Q), Keys (K), Values (V)

Transformer models are based on the principle of self-attention. Self-attention allows each element of the input sequence to look at all other elements in the input sequence and search for clues that can help it to create a more meaningful encoding. It is a way to look at which other sequence elements are relevant for the current element. The Transformer can grab context from both before and after the currently processed element.

When performing self-attention, three vectors need to be created for each element of the encoder input: the query vector (Q), the key vector (K), and the value vector (V). These vectors are created by performing matrix multiplications between the input embedding vectors using three unique weight matrices.

After this, self-attention scores are calculated. When calculating self-attention scores for a given element, the dot products between the query vector of this element and the key vectors of all other input elements are calculated. To make the model mathematically more stable, these self-attention scores are divided by the root of the size of the vectors. This has the effect of reducing the importance of the scalar thus emphasizing the importance of the direction of the vector. Just as before, these scores are normalized with a SoftMax layer. This attention distribution is then used to calculate a weighted sum of the value vectors, resulting in a vector z for every input element. In the attention principle explained above, the vector to calculate attention scores and to perform the weighted sum was the same, in self-attention two different vectors are created and used. As the self-attention needs to be calculated for all elements (thus a query for every element), one formula can be created to calculate a Z matrix. The rows of this Z matrix are the z vectors for every sequence input element, giving the matrix a size length sequence dimension QKV.

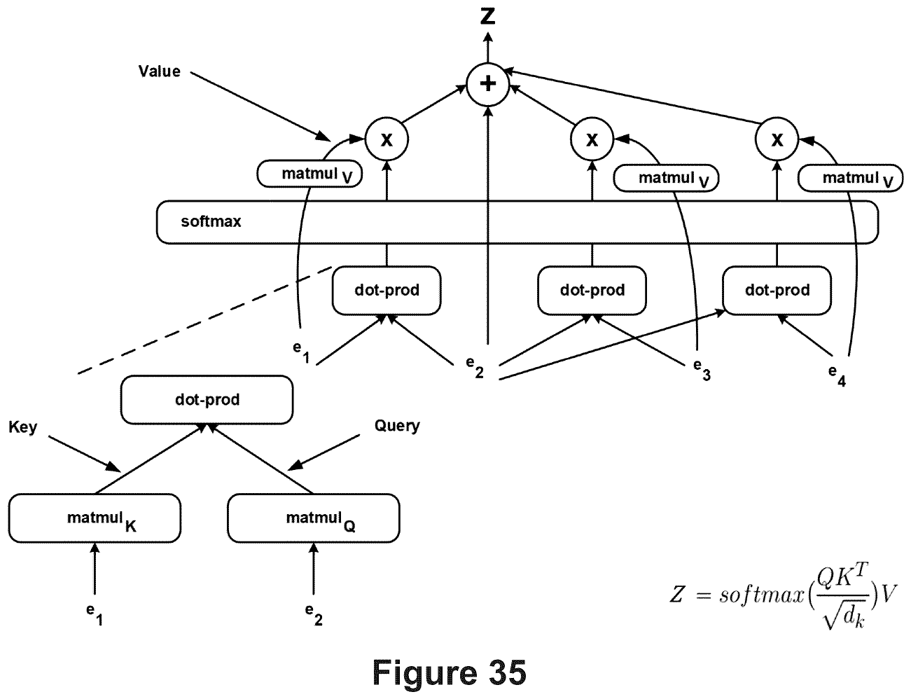

Multi-headed attention is executed in the Transformer. FIG. 35 is a schematic representation of the calculation of self-attention showing one attention head. For every attention head, different weight matrices are trained to calculate Q, K, and V. Every attention head outputs a matrix Z. Different attention heads can capture different types of information. The different Z matrices of the different attention heads are concatenated. This matrix can become large when multiple attention heads are used. To reduce dimensionality, an extra weight matrix W is trained to condense the different attention heads into a matrix with the same size as one Z matrix. This way, the amount of data given to the next step does not enlarge every time self-attention is performed.

When performing self-attention, information about the order of the different elements within the sequence is lost. To address this problem, positional encodings are added to the embedding vectors. Every position has its unique positional encoding vector. These vectors follow a specific pattern, which the Transformer model can learn to recognize. This way, the model can consider distances between the different elements.

As discussed above, in the core of self-attention are three objects: queries (Q), keys (K), and values (V). Each of these objects has an inner semantic meaning of their purpose. One can think of these as analogous to databases. We have a user-defined query of what the user wants to know. Then we have the relations in the database, i.e., the values which are the weights. More advanced database management systems create some apt representation of its relations to retrieve values more efficiently from the relations. This can be achieved by using indexes, which represent information about what is stored in the database. In the context of attention, indexes can be thought of as keys. So instead of running the query against values directly, the query is first executed on the indexes to retrieve where the relevant values or weights are stored. Lastly, these weights are run against the original values to retrieve data that is most relevant to the initial query.

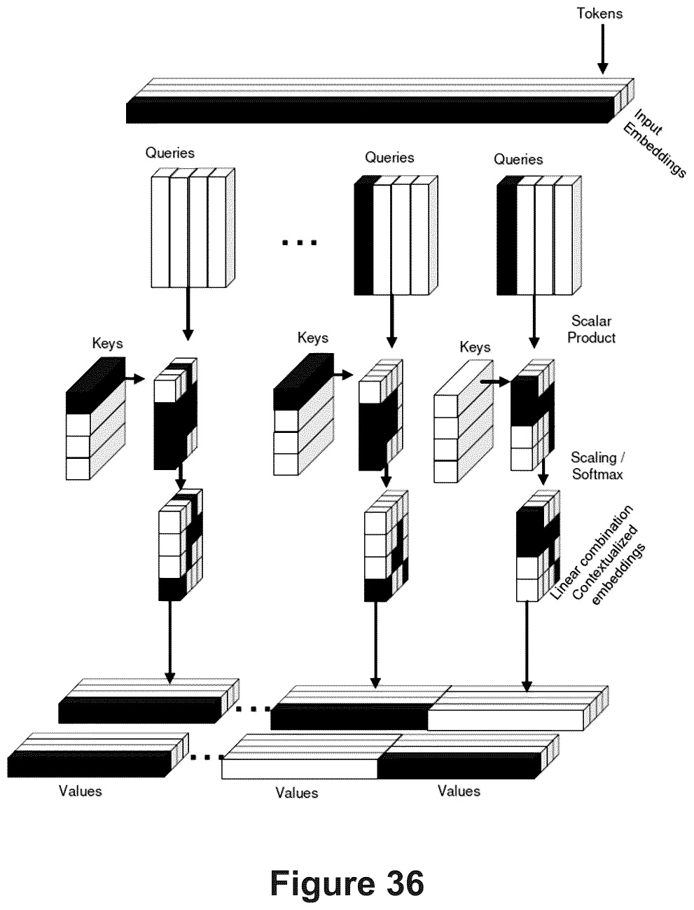

FIG. 36 depicts several attention heads in a Transformer block. We can see that the outputs of queries and keys dot products in different attention heads are differently colored. This depicts the capability of the multi-head attention to focus on different aspects of the input and aggregate the obtained information by multiplying the input with different attention weights.

Examples of attention calculation include scaled dot-product attention and additive attention. There are several reasons why scaled dot-product attention is used in the Transformers. Firstly, the scaled dot-product attention is relatively fast to compute, since its main parts are matrix operations that can be run on modern hardware accelerators. Secondly, it performs similarly well for smaller dimensions of the K matrix, dk, as the additive attention. For larger dk, the scaled dot-product attention performs a bit worse because dot products can cause the vanishing gradient problem. This is compensated via the scaling factor, which is defined as \sqrt{dk}.

As discussed above, the attention function takes as input three objects: key, value, and query. In the context of Transformers, these objects are matrices of shapes (n, d), where n is the number of elements in the input sequence and d is the hidden representation of each element (also called the hidden vector). Attention is then computed as:

$$Attention ( Q , K , V ) = SoftMax ( \frac{{QK}^{T}}{\sqrt{dk}} ) V$$

where Q, K, V are computed as:

$$X \cdot {W}_{Q} , X \cdot {W}_{K} , X \cdot {W}_{V}$$

X is the input matrix and W_{Q}, W_{K}, W_{V} are learned weights to project the input matrix into the representations. The dot products appearing in the attention function are exploited for their geometrical interpretation where higher values of their results mean that the inputs are more similar, i.e., pointing in the geometrical space in the same direction. Since the attention function now works with matrices, the dot product becomes matrix multiplication. The SoftMax function is used to normalize the attention weights into the value of 1 prior to being multiplied by the values matrix. The resulting matrix is used either as input into another layer of attention or becomes the output of the Transformer.

### Self-Attention: Queries (Q), Keys (K), Values (V)

Transformer models are based on the principle of self-attention. Self-attention allows each element of the input sequence to look at all other elements in the input sequence and search for clues that can help it to create a more meaningful encoding. It is a way to look at which other sequence elements are relevant for the current element. The Transformer can grab context from both before and after the currently processed element.

When performing self-attention, three vectors need to be created for each element of the encoder input: the query vector (Q), the key vector (K), and the value vector (V). These vectors are created by performing matrix multiplications between the input embedding vectors using three unique weight matrices.

After this, self-attention scores are calculated. When calculating self-attention scores for a given element, the dot products between the query vector of this element and the key vectors of all other input elements are calculated. To make the model mathematically more stable, these self-attention scores are divided by the root of the size of the vectors. This has the effect of reducing the importance of the scalar thus emphasizing the importance of the direction of the vector. Just as before, these scores are normalized with a SoftMax layer. This attention distribution is then used to calculate a weighted sum of the value vectors, resulting in a vector z for every input element. In the attention principle explained above, the vector to calculate attention scores and to perform the weighted sum was the same, in self-attention two different vectors are created and used. As the self-attention needs to be calculated for all elements (thus a query for every element), one formula can be created to calculate a Z matrix. The rows of this Z matrix are the z vectors for every sequence input element, giving the matrix a size length sequence dimension QKV.

Multi-headed attention is executed in the Transformer. FIG. 35 is a schematic representation of the calculation of self-attention showing one attention head. For every attention head, different weight matrices are trained to calculate Q, K, and V. Every attention head outputs a matrix Z. Different attention heads can capture different types of information. The different Z matrices of the different attention heads are concatenated. This matrix can become large when multiple attention heads are used. To reduce dimensionality, an extra weight matrix W is trained to condense the different attention heads into a matrix with the same size as one Z matrix. This way, the amount of data given to the next step does not enlarge every time self-attention is performed.

When performing self-attention, information about the order of the different elements within the sequence is lost. To address this problem, positional encodings are added to the embedding vectors. Every position has its unique positional encoding vector. These vectors follow a specific pattern, which the Transformer model can learn to recognize. This way, the model can consider distances between the different elements.

As discussed above, in the core of self-attention are three objects: queries (Q), keys (K), and values (V). Each of these objects has an inner semantic meaning of their purpose. One can think of these as analogous to databases. We have a user-defined query of what the user wants to know. Then we have the relations in the database, i.e., the values which are the weights. More advanced database management systems create some apt representation of its relations to retrieve values more efficiently from the relations. This can be achieved by using indexes, which represent information about what is stored in the database. In the context of attention, indexes can be thought of as keys. So instead of running the query against values directly, the query is first executed on the indexes to retrieve where the relevant values or weights are stored. Lastly, these weights are run against the original values to retrieve data that is most relevant to the initial query.

FIG. 36 depicts several attention heads in a Transformer block. We can see that the outputs of queries and keys dot products in different attention heads are differently colored. This depicts the capability of the multi-head attention to focus on different aspects of the input and aggregate the obtained information by multiplying the input with different attention weights.

Examples of attention calculation include scaled dot-product attention and additive attention. There are several reasons why scaled dot-product attention is used in the Transformers. Firstly, the scaled dot-product attention is relatively fast to compute, since its main parts are matrix operations that can be run on modern hardware accelerators. Secondly, it performs similarly well for smaller dimensions of the K matrix, dk, as the additive attention. For larger dk, the scaled dot-product attention performs a bit worse because dot products can cause the vanishing gradient problem. This is compensated via the scaling factor, which is defined as \sqrt{dk}.

As discussed above, the attention function takes as input three objects: key, value, and query. In the context of Transformers, these objects are matrices of shapes (n, d), where n is the number of elements in the input sequence and d is the hidden representation of each element (also called the hidden vector). Attention is then computed as:

$$Attention ( Q , K , V ) = SoftMax ( \frac{{QK}^{T}}{\sqrt{dk}} ) V$$

where Q, K, V are computed as:

$$X \cdot {W}_{Q} , X \cdot {W}_{K} , X \cdot {W}_{V}$$

X is the input matrix and W_{Q}, W_{K}, W_{V} are learned weights to project the input matrix into the representations. The dot products appearing in the attention function are exploited for their geometrical interpretation where higher values of their results mean that the inputs are more similar, i.e., pointing in the geometrical space in the same direction. Since the attention function now works with matrices, the dot product becomes matrix multiplication. The SoftMax function is used to normalize the attention weights into the value of 1 prior to being multiplied by the values matrix. The resulting matrix is used either as input into another layer of attention or becomes the output of the Transformer.

### Multi-Head Attention

Transformers become even more powerful when multi-head attention is used. Queries, keys, and values are computed the same way as above, though they are now projected into h different representations of smaller dimensions using a set of h learned weights. Each representation is passed into a different scaled dot-product attention block called a head. The head then computes its output using the same procedure as described above.

Formally, the multi-head attention is defined as:

MultiHeadAttention(Q,K,V)=[head₁, . . . ,headₕ]W₀ where head₁=Attention (QWᵢ^{Q},KWᵢ^{K},VWᵢ^{V})

The outputs of all heads are concatenated together and projected again using the learned weights matrix W₀ to match the dimensions expected by the next block of heads or the output of the Transformer. Using the multi-head attention instead of the simpler scaled dot-product attention enables Transformers to jointly attend to information from different representation subspaces at different positions.

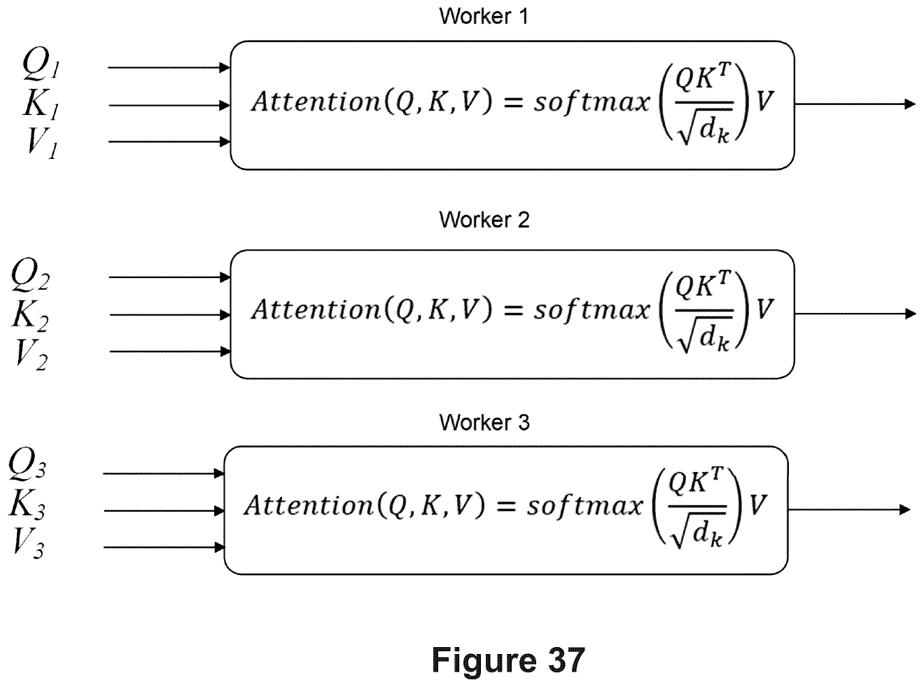

As shown in FIG. 37, one can use multiple workers to compute the multi-head attention in parallel, as the respective heads compute their outputs independently of one another. Parallel processing is one of the advantages of Transformers over RNNs.

Assuming the naive matrix multiplication algorithm which has a complexity of:

$$a \cdot b \cdot c$$

For matrices of shape (a, b) and (c, d), to obtain values Q, K, V, we need to compute the operations:

$$X \cdot {W}_{Q} , X \cdot {W}_{K} , X \cdot WV$$

The matrix X is of shape (n, d) where n is the number of patches and d is the hidden vector dimension. The weights W_{Q}, W_{K}, W_{V} are all of shape (d, d). Omitting the constant factor 3, the resulting complexity is:

n·d²

We can proceed to the estimation of the complexity of the attention function itself, i.e., of

$$SoftMax ( \frac{{QK}^{T}}{\sqrt{dk}} ) V .$$

The matrices Q and K are both of shape (n, d). The transposition operation does not influence the asymptotic complexity of computing the dot product of matrices of shapes (n, d)·(d, n), therefore its complexity is:

n²·d

Scaling by a constant factor of \sqrt{dk}, where dk is the dimension of the keys vector, as well as applying the SoftMax function, both have the complexity of a·b for a matrix of shape (a, b), hence they do not influence the asymptotic complexity. Lastly the dot product

$$SoftMax ( \frac{{QK}^{T}}{\sqrt{dk}} ) \cdot V$$

is between matrices of shapes (n, n) and (n, d) and so its complexity is:

n²·d

The final asymptotic complexity of scaled dot-product attention is obtained by summing the complexities of computing Q, K, V, and of the following attention function:

$$n \cdot {d}^{2} 4 {ln}^{2} \cdot d .$$

The asymptotic complexity of multi-head attention is the same since the original input matrix X is projected into h matrices of shapes

$$( n , \frac{d}{h} ) ,$$

where h is the number of heads. From the point of view of asymptotic complexity, h is constant, therefore we would arrive at the same estimate of asymptotic complexity using a similar approach as for the scaled dot-product attention.

Transformer models often have the encoder-decoder architecture, although this is not necessarily the case. The encoder is built out of different encoder layers which are all constructed in the same way. The positional encodings are added to the embedding vectors. Afterward, self-attention is performed.

### Encoder Block of Transformer

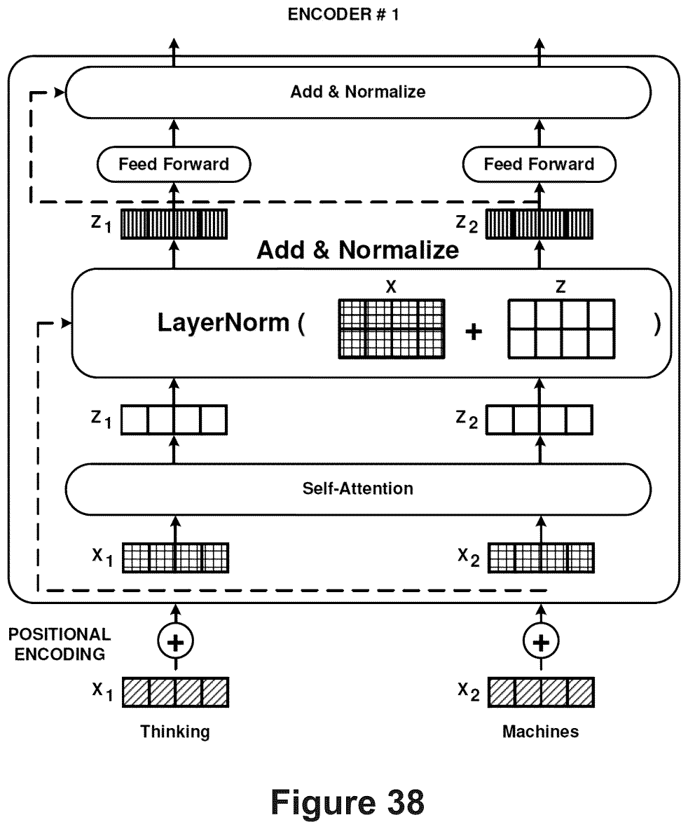

FIG. 38 portrays one encoder layer of a Transformer network. Every self-attention layer is surrounded by a residual connection, summing up the output and input of the self-attention. This sum is normalized, and the normalized vectors are fed to a feed-forward layer. Every z vector is fed separately to this feed-forward layer. The feed-forward layer is wrapped in a residual connection and the outcome is normalized too. Often, numerous encoder layers are piled to form the encoder. The output of the encoder is a fixed-size vector for every element of the input sequence.

Just like the encoder, the decoder is built from different decoder layers. In the decoder, a modified version of self-attention takes place. The query vector is only compared to the keys of previous output sequence elements. The elements further in the sequence are not known yet, as they still must be predicted. No information about these output elements may be used.

### Encoder-Decoder Blocks of Transformer

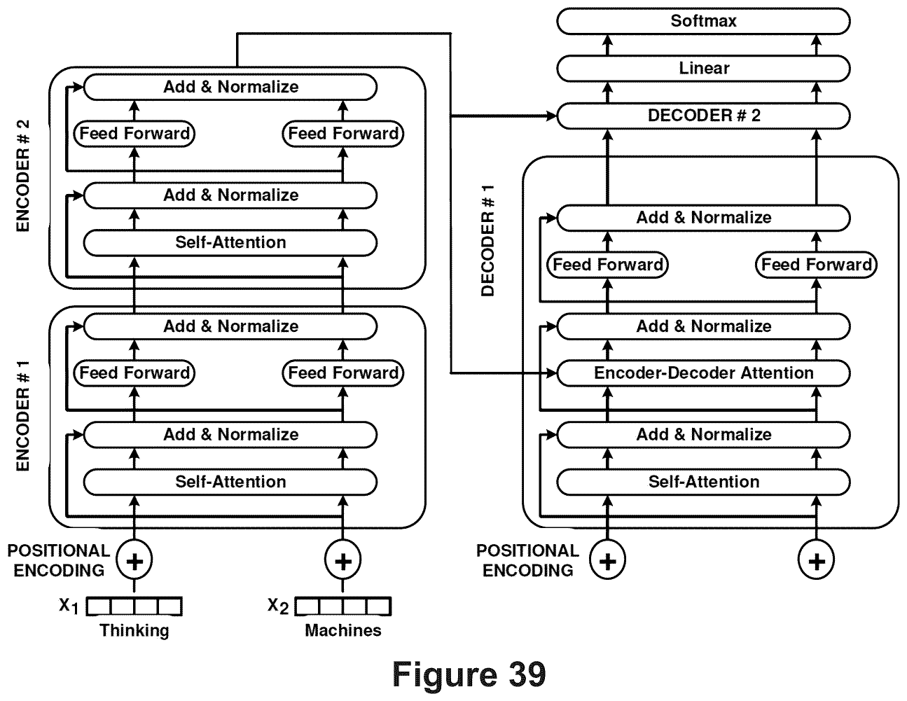

FIG. 39 shows a schematic overview of a Transformer model. Next to a self-attention layer, a layer of encoder-decoder attention is present in the decoder, in which the decoder can examine the last Z vectors of the encoder, providing fluent information transmission. The ultimate decoder layer is a feed-forward layer. All layers are packed in a residual connection. This allows the decoder to examine all previously predicted outputs and all encoded input vectors to predict the next output. Thus, information from the encoder is provided to the decoder, which could improve the predictive capacity. The output vectors of the last decoder layer need to be processed to form the output of the entire system. This is done by a combination of a feed-forward layer and a SoftMax function. The output corresponding to the highest probability is the predicted output value for a subject time step.

For some tasks other than translation, only an encoder is needed. This is true for both document classification and name entity recognition. In these cases, the encoded input vectors are the input of the feed-forward layer and the SoftMax layer. Transformer models have been extensively applied in different NLP fields, such as translation, document summarization, speech recognition, and named entity recognition. These models have applications in the field of biology as well for predicting protein structure and function and labeling DNA sequences.

### Vision Transformer

There are extensive applications of transformers in vision including popular recognition tasks (e.g., image classification, object detection, action recognition, and segmentation), generative modeling, multi-modal tasks (e.g., visual-question answering, visual reasoning, and visual grounding), video processing (e.g., activity recognition, video forecasting), low-level vision (e.g., image super-resolution, image enhancement, and colorization) and 3D analysis (e.g., point cloud classification and segmentation).

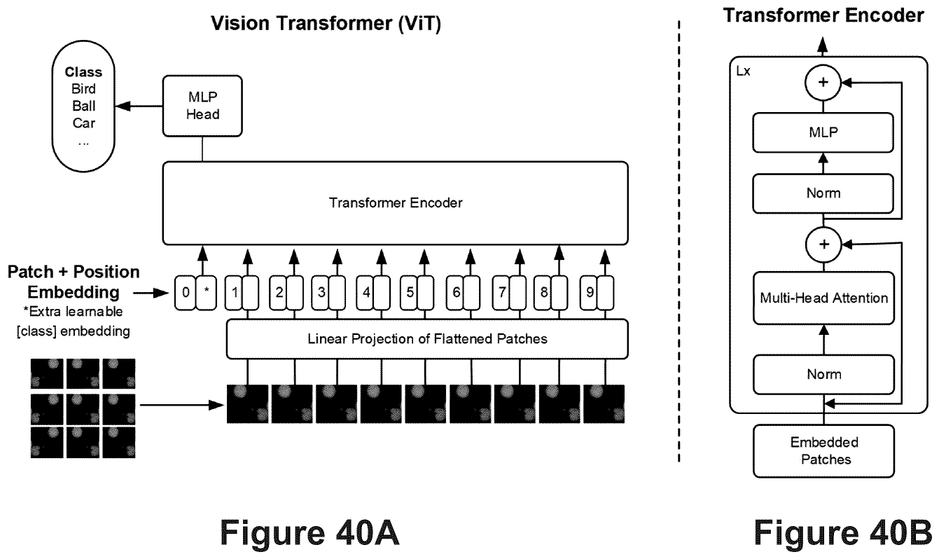

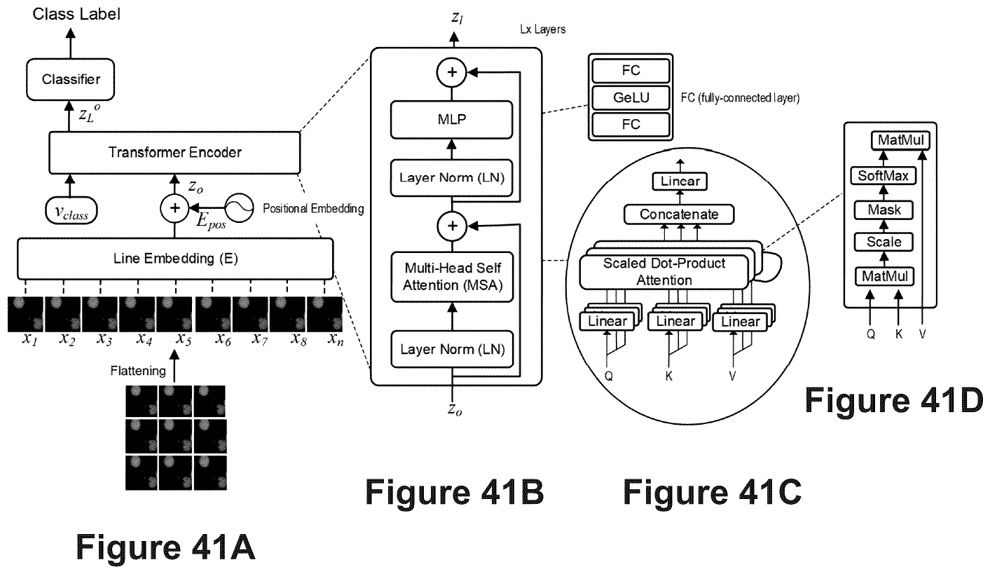

Transformers were originally developed for NLP and worked with sequences of words. In image classification, we often have a single input image in which the pixels are in a sequence. To reduce the computation required, Vision Transformers (ViTs) cut the input image into a set of fixed-sized patches of pixels. The patches are often 16×16 pixels. They are treated much like words in NLP Transformers. ViTs are depicted in FIGS. 40A, 40B, 41A, 41B, 41C, and 41D. Unfortunately, important positional information is lost because image sets are position-invariant. This problem is solved by adding a learned positional encoding into the image patches.

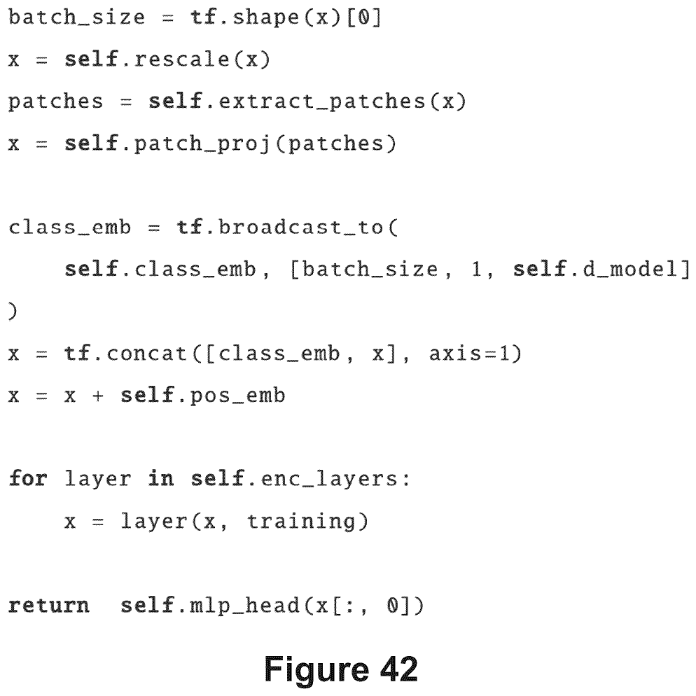

The computations of the ViT architecture can be summarized as follows. The first layer of a ViT extracts a fixed number of patches from an input image (FIG. 40A). The patches are then projected to linear embeddings. A special class token vector is added to the sequence of embedding vectors to include all representative information of all tokens through the multi-layer encoding procedure. The class vector is unique to each image. Vectors containing positional information are combined with the embeddings and the class token. The sequence of embedding vectors is passed into the Transformer blocks. The class token vector is extracted from the output of the last Transformer block and is passed into a multilayer perceptron (MLP) head whose output is the final classification. The perceptron takes the normalized input and places the output in categories. It classifies the images. This procedure directly translates into the Python Keras code shown in FIG. 42.

When the input image is split into patches, a fixed patch size is specified before instantiating a ViT. Given the quadratic complexity of attention, patch size has a large effect on the length of training and inference time. A single Transformer block comprises several layers. The first layer implements Layer Normalization, followed by the multi-head attention that is responsible for the performance of ViTs. In the depiction of a Transformer block in FIG. 40B, we can see two arrows. These are residual skip connections. Including skip connection data can simplify the output and improve the results. The output of the multi-head attention is followed again by Layer Normalization. And finally, the output layer is an MLP (Multi-Layer Perceptron) with the GELU (Gaussian Error Linear Unit) activation function.

ViTs can be pretrained and fine-tuned. Pretraining is generally done on a large dataset. Fine-tuning is done on a domain specific dataset.

Domain-specific architectures, like convolutional neural networks (CNNs) or long short-term memory networks (LSTMs), have been derived from the usual architecture of MLPs and suffer from so-called inductive biases that predispose the networks towards a certain output. ViTs stepped in the opposite direction of CNNs and LSTMs and became more general architectures by eliminating inductive biases. A ViT can be seen as a generalization of MLPs because MLPs, after being trained, do not change their weights for different inputs. On the other hand, ViTs compute their attention weights at runtime based on the particular input.

Ageing is a gradual biological process characterized by functional decline in both physical and cognitive capabilities. It presents a major risk factor for age-related disorders such as osteoarthritis, cancer, diabetes, cardiovascular and neurodegenerative diseases, and ultimately increased risk of death. The past two decades, a growing interest in longevity (lifespan) and healthy ageing (healthspan) led to tremendous efforts to unravel the biological basis with the hope to delay or prevent age-related conditions and increase both life- and healthspan. Ageing is regulated by complex cellular and molecular mechanisms, and variation in life- and healthspan between humans is affected by a variety of factors including genetic background but also socioeconomic and environmental elements.

Epigenetics connects the genotype to the phenotype and plays an important part in the response to environmental factors hinting at a lead role in modulating the ageing process. Multiple studies indeed have shown the link between rate of ageing and alterations in epigenetic regulators such as DNA-methylation (DNAm), histone modifications, non-coding RNAs and chromatin organization. As epigenetic modifications are often reversible, these findings greatly facilitated the emerging anti-ageing field and the development of ageing-delaying or even reverse-ageing therapies. Especially DNAm levels have been the focus of many age-related studies as DNAm has been well-reported to dynamically change with age. For example, DNAm levels tend to increase with age at some CpG islands whereas loci outside CpG islands usually lose methylation.

Interestingly, these studies further showed that the DNA methylome, and in particular specific collections of individual 5-methylcytosine sites, can be used to predict chronological age of a variety of tissues, and in 2013 Horvath introduced the concept of the “epigenetic or DNAm clock”. Furthermore, apart from being a predictor for chronological age, DNAm may also serve as a valuable biomarker for healthy versus unhealthy ageing and disease risk, and thus a proxy for “biological age”. Importantly, while chronological age is independent of genetic background, lifestyle, health and disease, biological age can be highly variable between people of the same chronological age. Indeed, disparity between an individuals' chronological and biological age may reflect the impact of specific genetic and environmental factors, leading to an acceleration or deceleration of age-related functional decline compared to peers of the same chronological age. Multiple methods have been proposed to measure biological age, including physiological (e.g., blood, cardiovascular, cognitive, and physical parameters) as well as some molecular (e.g., telomere length, mitochondrial function) markers. However, despite its potential as an objective quantification of healthspan, there is currently no consensus on the best method to determine biological age, mainly reflecting its inherent complexity. However, some recent studies have stipulated that the delta between one's DNAm age and true chronological age, might be a surrogate for biological age. In this regard, a positive delta indicates an DNAm age-acceleration resulting in a biologically older individual, while a negative delta reflects that the individual aged slower and is biologically younger. For example, one study investigating blood of centenarians and their offspring reported a negative delta, i.e., DNAm age-deceleration, for this population.

In light of these findings, there has been huge excitement in the use of DNAm age. This, together with the advances in DNAm arrays and the data availability, enabled the creation of several methylation clocks. The first methylation clocks were created for humans but have recently also been developed for other organisms like mice and dogs, amongst others. The best-known human DNAm clocks are probably those from Horvath (multi-tissue), Hannum (blood), Levine (PhenoAge, blood) and Lu (GrimAge, blood) but many others exist. Here, the former two are referred to as first-generation clocks because they were trained to predict chronological age. The latter two clocks, on the other hand, are labelled second-generation clocks, because they were trained on a surrogate biomarker reflecting ageing phenotypes to better reflect disease morbidity and mortality with the aim to predict biological age.

While each clock slightly differs in the used datasets and/or data processing approach, the vast majority are linear models of a set of CpGs, with or without intercept, regressing to either chronological age or to a proxy for biological age. Each linear model is typically built using the same general supervised workflow, namely by leveraging a penalized or regularized regression model like ElasticNet or Lasso to automatically select the most informative set of CpGs for the prediction out of the full methylation array after which a linear model is developed on those CpGs. The final number of selected CpGs differs for each linear model and usually ranges between 3 and 2000. For example, the Horvath, Hannum, Levine, and Lu clocks contain 353, 71, 513 and 1,113 CpGs respectively. While regularized linear regression has been widely used to develop epigenetic clocks, there are some important limitations with this approach. Not only can linear regression display high bias, but it is also not able to capture more complex, non-linear CpG interactions. More importantly, epigenetic clocks based on linear models assume that the contribution of each CpG is additive and that the rate of methylation change is constant over the entire lifespan. In addition, regularized regression methods are often sensitive to subtle differences in the used data. This is evidenced by the fact that many of the published clocks use completely different, non-overlapping sets of CpGs, even when trained for the same tissue, making it difficult to assess any biological or functional connection to the ageing process.

Deep learning (DL) has proved to be a powerful and flexible approach for many data types, including molecular data. When provided with large, high-dimensional data, neural networks (NN) are able to capture complex linear and non-linear feature interactions. Also, for DNAm data and DNAm age prediction, NN models have already been successfully applied. For example, methods like DeepMAge and AltumAge use a deep NN for blood and pan-tissue DNAm age prediction, respectively. By using a multi-layer perceptron (MLP) architecture, both methods outperform regularized linear regression models such as Horvath in chronological age prediction, seem to be more resilient to noise and generalize better to new data. Furthermore, they illustrate that NNs allow to simultaneously use of information of thousands of CpGs, can capture higher-order, complex feature interactions and that it is possible to determine the contribution of each CpGs by leveraging DL interpretation methods like Shapley Additive Explanation (SHAP). As such, these results show that DL is a very encouraging approach for epigenetic clock predictions and to unravel complex biological mechanisms within the context of ageing.

Among DL models, variational autoencoders (VAE) are an emerging technique that hold great potential in a wide range of applications. VAEs are data driven, unsupervised generative models harness the power of DL and learn data distributions without the need for accurate labels. The underlying architecture consists of autoencoding layers which first encode (compress) the data in a lower-dimensional latent space after which the data is decoded (reconstructed) into its original dimension. Here, the aim of the encoder phase is not only to reduce the data dimensionality, but to compress the data by removing redundant information while at the same time keeping the most important information relevant for the research question in this reduced representation. While a traditional AE minimizes the reconstruction error during training and results in a non-regularized latent space (the decoder cannot be used to generate valid input data from vectors sampled from the latent space), a VAE is stochastic and learns the parameters of the data distribution, i.e., mean and standard deviation, ensuring a latent space with generative capabilities. Importantly, the fact that the autoencoding makes use of complex, non-linear activation functions, allows the VAE to learn complex, lower-dimensional representations of the data. Because of the ability to apprehend underlying data manifolds, VAEs have been employed to generate meaningful biological latent spaces various biological applications. For instance, Way et al. trained a VAE on TCGA pan-cancer RNA-seq data to model cancer gene expression and observed that the encoded lower-dimensional features contained biological meaningful signals. Similarly, Kinalis et al. show that it is possible to directly deconvolute biological modules inherent in single cell RNA-seq data that outline cell-specific drivers, without making any prior assumptions. Of late, VAEs are being used to reduce the dimensionality of DNAm data. Methylation profiles are typically measured via DNAm microarrays such as Illumina's HumanMethylation27 (27K), HumanMethylation450 (450K) and the most recent HumanMethylationEPIC (850K), measuring approximately 27,000, 450,000 and 850,000 CpG sites, respectively, and are reported as continuous beta-values ranging from 0 (no methylation), to 1 (fully methylated). Given the high number of features, VAEs have been applied to embed this data to into a lower-dimensional meaningful feature set. For example, Levy et al. investigated the use of VAEs on six different DNAm datasets and demonstrated its potential to capture cancer subtype information, cellular differences, and smoking factors, and to make age predictions. Two other DNAm breast cancer studies illustrated the use of the encoded latent space to predict cancer subtypes. In addition, when comparing to regularized regression-based methods for feature selection, VAEs were able to reveal novel information about DNAm patterns in breast cancer.

Here, we explore the use of VAEs to create a biologically relevant latent space that can be used to extract relevant biological signals related to ageing but also to model DNAm clocks to predict biological age. For this, we developed framework that first performs a prior dimensionality reduction to ˜13 k CpGs which are fed into a VAE model. In a next step, the learned latent space is used as input of an MLP to predict age. We trained our framework using 8 publicly available DNAm 450K datasets originating, totaling 2,716 blood samples from healthy individuals with chronological ages ranging between 0-103. We present initial results from using VAEs as a dimensionality reduction method for DNAm ageing applications and show that this lower dimensional latent space holds relevant information that may lead to mechanistic hypotheses on how the methylome drives the ageing process. We further demonstrate that this learned latent space can subsequently be used as input features for models to estimate age. Additional validation on two independent cohort not only illustrates that the developed models can be used to calculate DNAm age, but that the VAE has the potential to be used to distinguish diseased from health samples for age-related disorders.

Taken together, our analysis hints towards more robust DL-based methylation clocks with better estimates for biological age that at the same time are able to unravel complex biological networks involved in ageing and/or age-related disorders. It is our expectation that in the future, VAEs or other DL feature selection methods that can capture complex biological functions pave the way to combine multiple modalities relevant for ageing, e.g., molecular (DNAm, gene expression, metabolic profiles—single or multi-tissue), image data as well as socioeconomic factors and health records in one multimodal model for biological age prediction.

Our results indicate that VAEs present a promising framework to construct embeddings that capture complex interactions and that can be used to extract biological meaningful features upon predicting DNAm age. By using deep learning interpretation methods, we show it is possible to determine which sites and pathways are important in making the predictions, both on a population and individual level, paving the way to unravel what makes the DNAm clock tick.

The term “epigenetic” and the phrase “non-sequence altering change” are used interchangeably in this patent application.

The term “methylation” and the term “modification” are used interchangeably in this patent application.

The term “disease” and the term “condition” are used interchangeably in this patent application.

### Data & Preprocessing

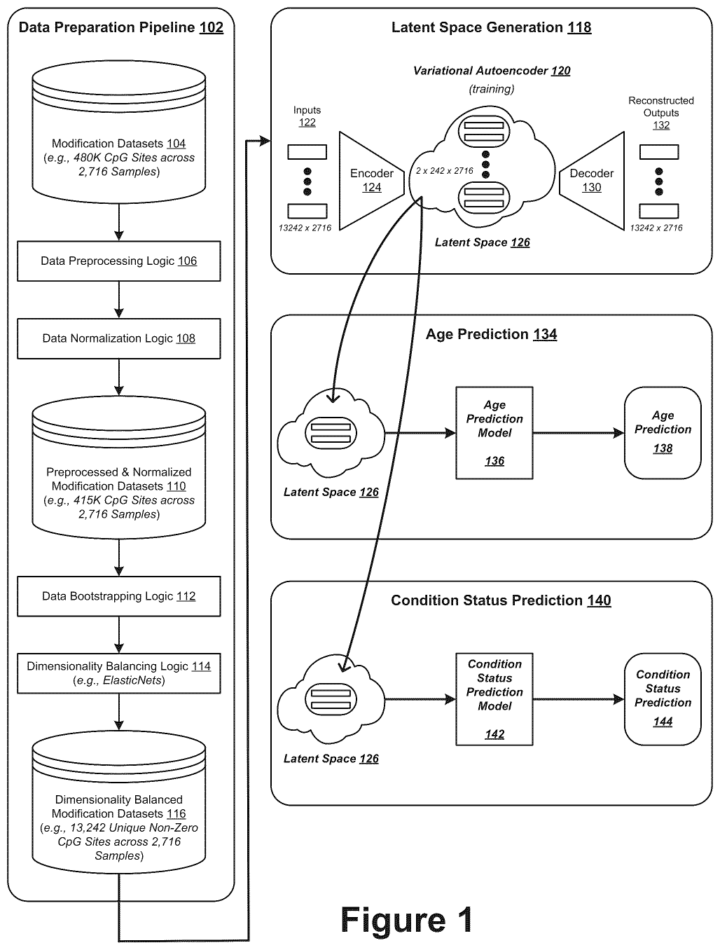

FIG. 1 shows one implementation of a data preparation pipeline 102, comprising components 104, 106, 108, 110, 112, 114, and 116.

Methylation data used in this study consisted of 2,716 blood samples measured on Illumina's Infinium HumanMethylation450 BeadChip (450K) array originating from seven public datasets. Raw data was downloaded from the Gene Expression Omnibus (GEO) (six datasets, i.e., GSE30870, GSE32149, GSE41169, GSE36064, GSE40279, GSE87571) and ArrayExpress (SATSA dataset—E-MTAB-7309). Details on the individual datasets and characteristics of the specific study cohort can be found in Table 1. A full description of each dataset can be found in the original reference.

TABLE 1
Overview of used 450K methylation datasets:

| | #Samples | | Median Age | |
|---|---|---|---|---|
| Dataset | (origin) | Description | (min-max) | |
| GSE30870 | 40 (blood | The study aimed to compare the DNAm | 44.5 | (0-103) |
| | PBMC) | differences between newborns and | | |
| | | nonagenarians using methylation array | | |
| | | technology | | |
| GSE32149 | 46 (blood | DNAm study of Crohn's disease (n = 14), | 15 | (3.5-76) |
| | PBMC) | ulcerative colitis (n = 8) and controls | | |
| | | (n = 14). DNAm age was not found to be | | |
| | | associated with disease status. | | |
| GSE41169 | 95 (whole | Genome wide DNAm profiling of whole | 29 | (18-65) |
| | blood) | blood in schizophrenia patients and healthy | | |
| | | subjects of different ages. Dataset included | | |
| | | 62 schizophrenia patients and 33 healthy | | |
| | | subjects from Dutch descent. | | |
| GSE36064 | 78 (blood | DNAm association study in healthy | 3.1 | (1-16) |
| | PBMC) | children (males) | | |
| GSE40279 | 656 (whole | Genome wide DNAm profiling of healthy | 65 | (19-101) |
| | blood) | individuals across a large age range. | | |
| GSE87571 | 729 (whole | Genome wide DNAm profiling of healthy | 47 | (14-94) |
| | blood) | individuals across a large age range. | | |
| SATSA | 1072 (blood | DNAm of longitudinal samples from The | 73.5 | (48-99) |
| | PBMC) | Swedish Adoption/Twin Study of Ageing | | |
| | | (SATSA). ~400 Swedish twins were | | |
| | | sampled over 20 years (up to 5 times per | | |
| | | sample). | | |

The Illumina 450K array measures bisulfite-conversion-based, single-CpG resolution DNA methylation levels for over 480K CpG sites and covers 96% of CpG islands in the human genome. Unlike the previous 27K platform, the Illumina 450K array includes two distinct probe types, i.e., Infinium I (n=135,501) and Infinium II (n=350,076). In the Infinium type, each CpG cute is targeted by two 50 bp probes: one for detecting the methylated intensity (M) and one for detecting the unmethylated intensity (U). In contrast, Infinium II probes detect both the M and U intensity of each CpG site by one single probe using different dye colors (green and red). For each CpG site, methylation values are indicated by the beta-value which ranges from 0 (no methylation) to 1 (fully methylated) and is calculated as beta=M/(M+U+alpha) where alpha generally equals 100.

Raw beta-values were processed in R (v4.1.2) with the RnBeads package (v2.12.2). Probes not in CpG context were filtered out as well as probes for which the beta-values were NA or had low variability (standard deviation<0.005). Beta values of the remaining probes were next normalized using the Beta Mixture Quantile dilation method (BMIQ) with an Exponential-Normal mixture (Enmix) signal intensity background correction. After preprocessing and normalization, only probes present in all eight datasets were kept resulting in a final dataset of 2,716 samples and 415,594 CpG probes. Table 2 summarizes the number of samples per age group for this population.

TABLE 2
Number of samples per age group with their respective weights:

| Age Group | # Samples | Group weight |
|---|---|---|
| [0, 20] | 239 | 0.132 |
| [20, 30] | 142 | 0.222 |
| [30, 40] | 136 | 0.232 |
| [40, 50] | 221 | 0.143 |
| [50, 60] | 327 | 0.092 |
| [60, 70] | 529 | 0.057 |
| [70, 80] | 594 | 0.050 |
| 80+ | 444 | 0.071 |
| Total | 2,716 | 1 |

### Dimensionality Reduction

Typically, methylation arrays inherently present a dimensionality imbalance (i.e., number of CpGs (p)<<number of samples (n)). To handle the dimensionality imbalance in our dataset (p=415,594, n=2,716), a prior feature selection step was performed using ElasticNets, a shrinkage technique that uses a weighted combination of L1 (Ridge) and L2 (Lasso) regularization. As shown in Table 2, the predictor variable age is not uniformly distributed our dataset, i.e., the samples are imbalanced between different age classes. Therefore, before fitting an ElasticNet, samples were bootstrapped with a fraction of 42% and sample weights equal to the respective age class weight (Table 2).

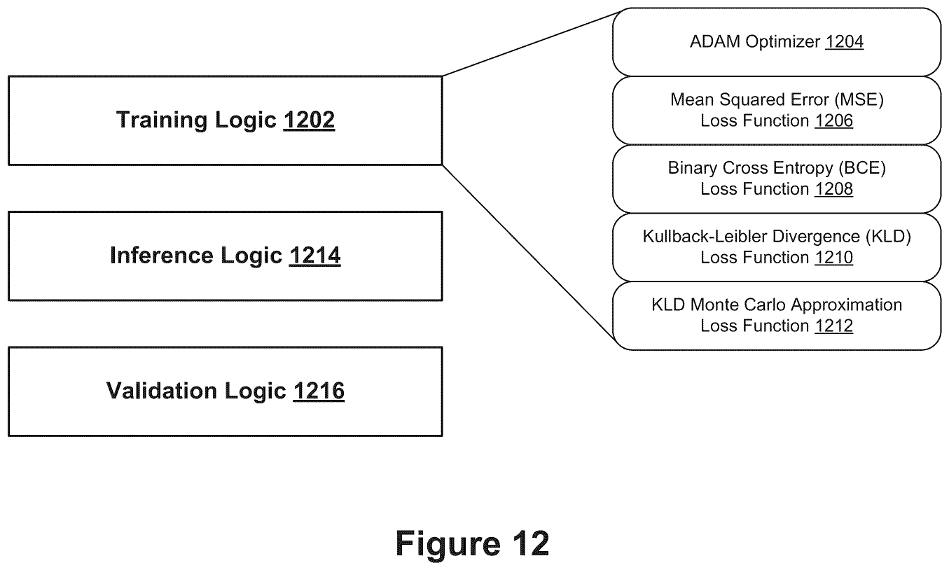

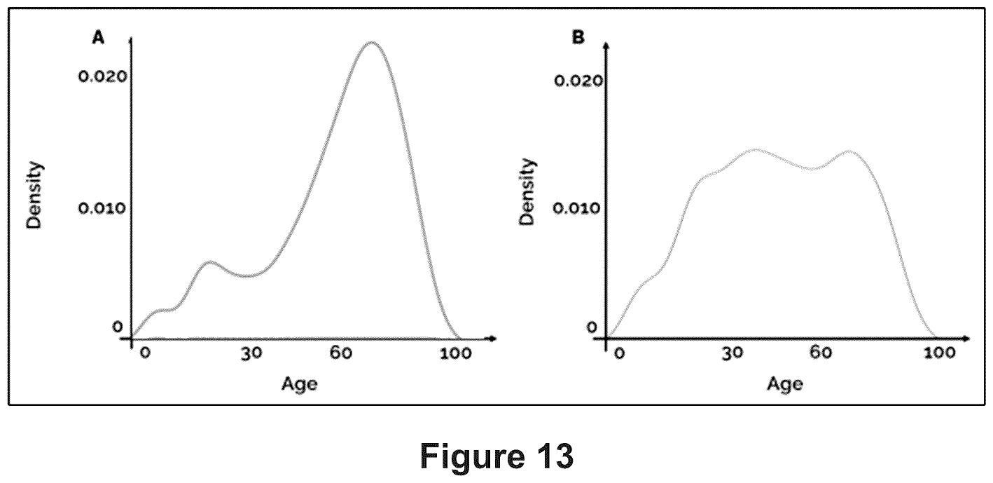

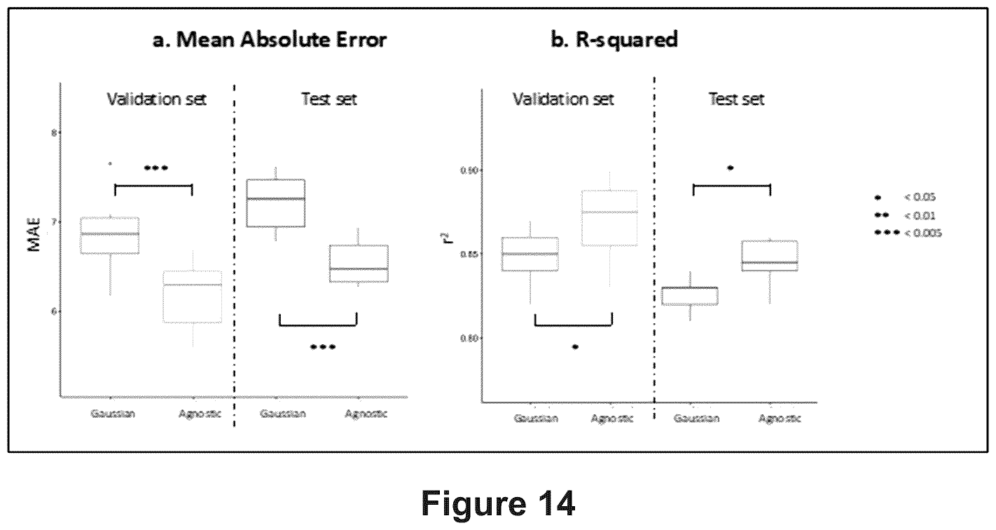

FIG. 12 shows different logics and functions of the technology disclosed, including logics 1202, 1214, and 1216, and functions 1204, 1206, 1208, 1210, and 1212. FIG. 13 shows the original age density distribution (panel A) and the resulting density distribution of one such bootstrap (panel B).

This bootstrapping procedure was performed 42 times, and for each bootstrapped distribution an ElasticNet was fitted with an L1/L2 ratio of 0.5 and the non-zero coefficients stored. On average, each ElasticNet had 895 non-zero coefficients with a range of [821; 955]. In a next step, the non-zero coefficients from all bootstrap iterations were combined, resulting in 13,242 unique non-zero coefficients, i.e., CpGs, out of the initial 415,594 (=3.19%).

FIG. 1 shows one implementation of latent space generation 118, comprising components 120, 122, 124, 126, 130, and 132.

FIG. 1 shows one implementation of age prediction 134, comprising components 126, 136, and 138.

FIG. 1 shows one implementation of condition status prediction 140, comprising components 126, 142, and 144.

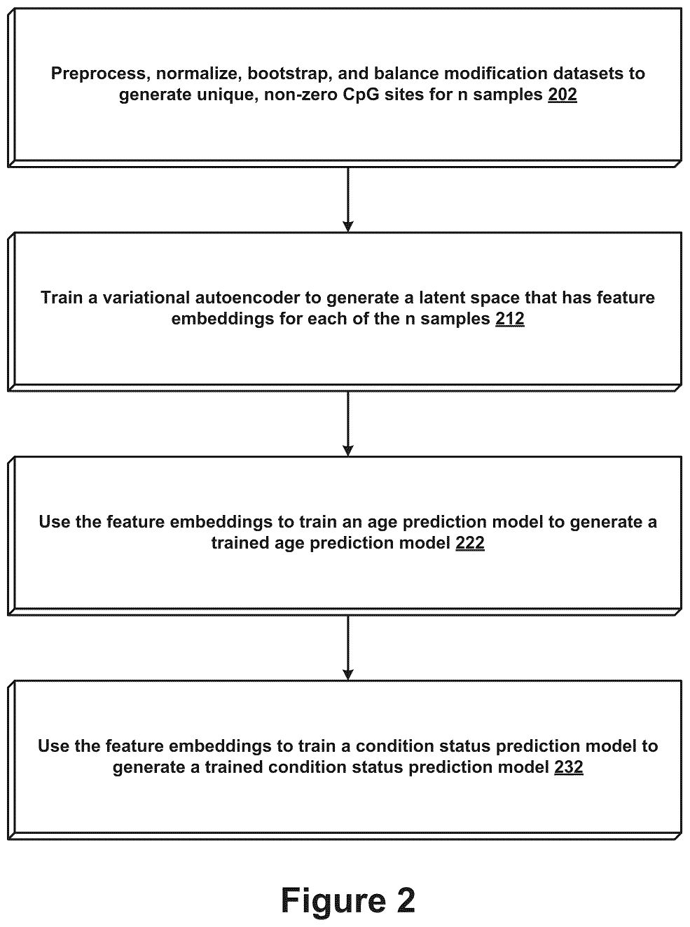

FIG. 2 is a flowchart showing one method implementation of the technology disclosed, including steps 202, 212, 222, and 232.

### Variational Autoencoder Model

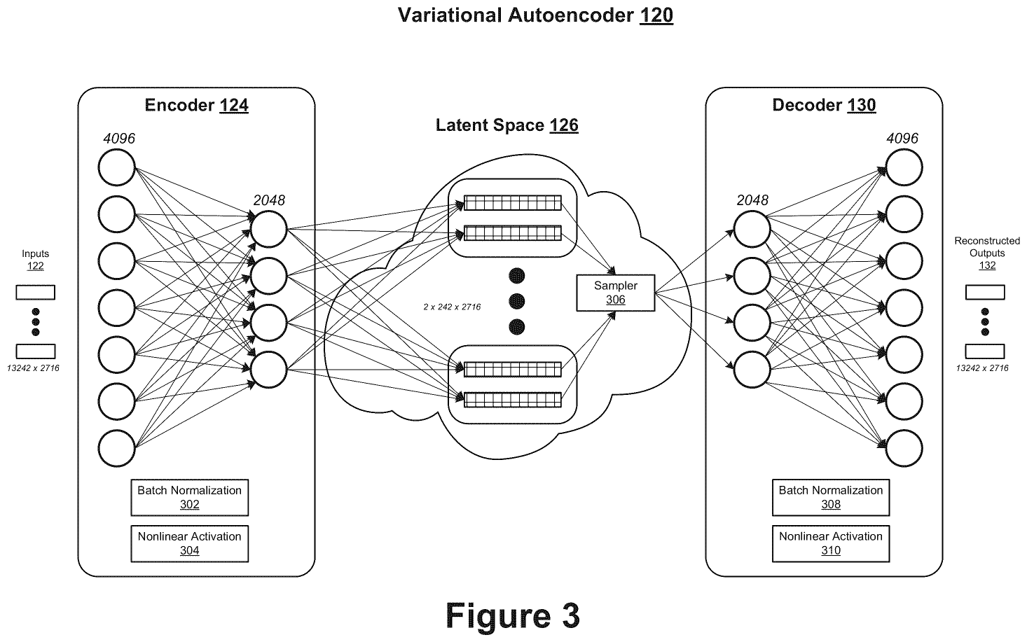

We employed a variational autoencoder (VAE), an unsupervised deep learning architecture that consists of two neural network parts: an encoder and a decoder (FIG. 3), in conjunction with components 302, 304, 306, 308, and 310. The encoder converts the input (our M×N data matrix with M=2,716 samples, and N=13,242 CpGs) to a lower dimensional latent space consisting of two feature vectors representing the input as a jointly Gaussian distribution. The decoder then randomly samples from this latent distribution to regenerate the data. Here, the effectiveness of the model is evaluated by comparing reconstructed data to the original input data. Importantly, the latent space (also called embedding) is the key concept of the VAE: each of the input features is combined in a weighted (nonlinear) pattern that contributes to the learned latent representation. Hence the encoder can be seen as a complex feature extractor selecting relevant signals in the data while removing the noise.

We constructed a VAE with 2 linear layers for encoding with dimensions 4096 and 2048 respectively, a latent space of size 242 and a two linear decoding layers with dimensions 2048 and 4096, respectively (FIG. 3). Between each linear layer, a 1D batch normalization was performed and the non-linear ReLu function was used as activation function. The input of the VAE was limited to the 13,242 CpGs selected by the ElasticNet analysis. The VAE model was trained using the ADAM optimizer with two loss functions: (i) a Mean Squared Error (MSE) loss function capturing the reconstruction loss and (ii) a Kullback-Leibler divergence (KLD) loss function measuring the distance between two distributions. Two KLD implementations were tested: (i) one that determines the KLD between the latent distribution and a Gaussian distribution, thereby assuming hence forcing the sampling in the representation layer to approximate a normal random variable, and (ii) a Monte Carlo approximation of KLD which is distribution agnostic, i.e., without making any prior assumptions about the underlying distribution. The total training loss was defined as: MSEloss+w*KLDloss with w the weight of the KLD term. Also, here two setups were tested: (i) one where w=1 and (ii) one where w varies between 0-1 following a cyclical sigmoid annealing scheduling to mitigate KLD-vanishing.

Samples were shuffled in a training and test set at an 80/20 ratio with stratification on age class. This test set was left out during model training and only used for final model performance. A 10-fold cross validation (CV) performed on the training set. Hyperparameters were empirically determined. Each CV model was trained for 100 epochs using a batch size of 64, a learning rate of 1e-3. To prevent exploding gradients during VAE training, a weight decay (L2 regularization) of 1e-4 was set and gradients were clipped with a norm of 0.01. For each CV, the weights were saved for the epoch with the lowest loss on the validation set. The best model was chosen based on the CV configuration with the highest validation accuracy. This final model was next evaluated on the test set.

### Multi-Layer Perceptron

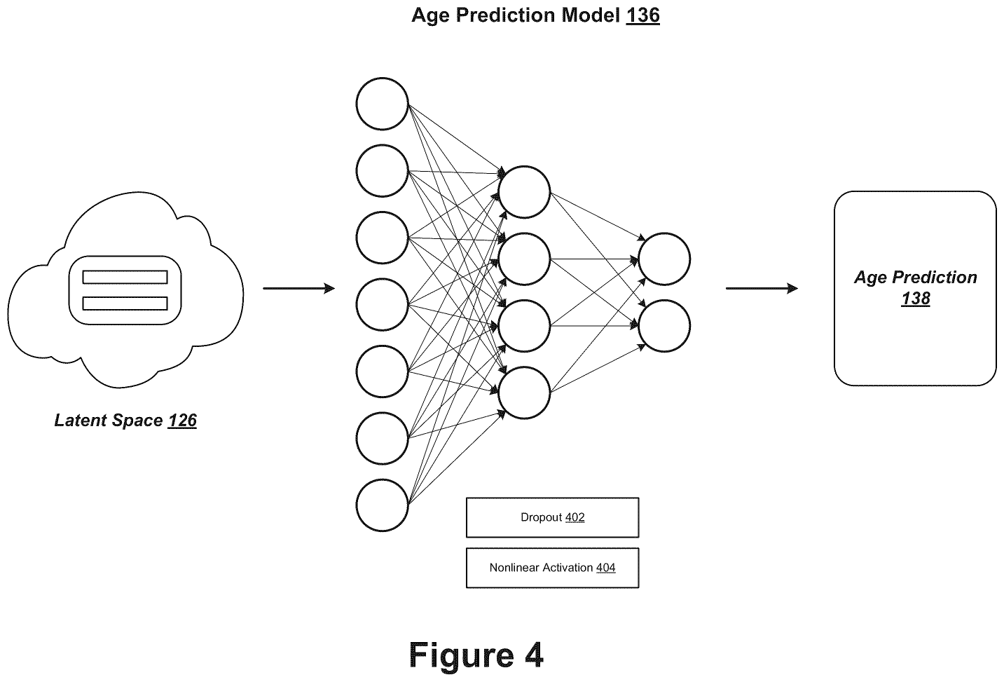

In order to test the utility of the learned latent methylation space to predict age, a three-layer multi-layer perceptron was trained with input layer of size 242 (=size of latent space), 3 hidden layers of size 128, 64 and 32 and output layer of size 1 (FIG. 4), in conjunction with components 402 and 404. Before each layer, a dropout with probability 0.42 was implemented and the non-linear ReLu function was used as activation function. As for the VAE, the ADAM optimizer was used together with a weighted MSE loss function, where the error on each sample was weighted according to its age class weight (Table 2). The MLP was trained for 100 epochs, using a batch size of 32, learning rate of 1e-3 and weight decay of 1e-4. Also here, the best model was selected as the epoch with the highest performance on the validation set and evaluated on the test set.

### Model Validation

To evaluate the VAE and MLP models, additional validation was done on two independent datasets. A first dataset consisted of 418 DNAm samples originating from an African American sibling cohort from the Genetic Epidemiology Network of Arteriopathy (GENOA) study (GEO accession GSE210254). DNAm age was calculated for each sample, and performance evaluated by calculating the mean absolute error (MAE) and r2. Results were further compared to Horvath, Hannum and PhenoAge DNAm age predictions.

A second dataset consisted of 689 peripheral blood leukocytes samples from a DNAm study to determine whether rheumatoid arthritis (RA) patients have methylation differences comparing to normal controls (GEO accession GSE42861). Here, although with a small effect size (age difference of 1.2 years), the researchers found a significant (p=0.0049) difference between the two groups. While Horvath did not find a significant difference of negative age acceleration between the diseased versus healthy samples, we wanted to evaluate if our models could pick up any clinical signal or deviation in RA patients. To test this, we first computed a universal measure of age acceleration (AgeAcc) as the DNAm age predicted by our model divided by chronological age. We also calculated AgeAcc using DNAm age predicted by Horvath, Hannum and PhenoAge clocks.

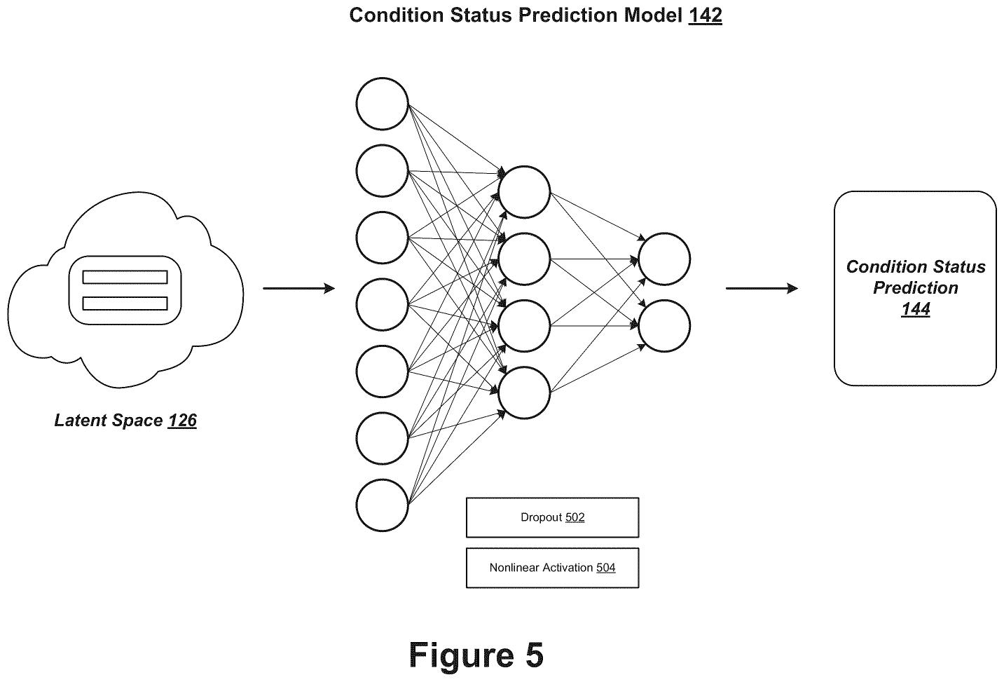

To assess if the latent space encoded by our VAE can be used as a biological feature extractor for age-related features, we trained a simple logistic regression model to predict RA status (FIG. 5), in conjunction with components 502 and 504. The dataset was split in an 80/20 ratio with stratification on RA status and a Logistic Regression (Logit) model was trained on the training set using sklearn's LogisticRegressionCV function with L1 penalty and the saga solver. Performance was measured on the test set by calculating the confusion matrix and generating the ROC curves.

These datasets were not used in any training or fine-tuning step but kept as a separate, independent cohorts for final model validation.

While DL has the ability to extract predictive features from complex data, these are usually abstract. Here, we propose a method that leverages the VAE to biologically interpret the latent space in a post-hoc manner. The relation with the input features (13,242 CpGs) was determined by backpropagation of the VAE model and calculating the model gradients. The importance of each gene was next assessed by averaging the gradients of the gene-associated CpGs. Likewise, the importance of each pathway in each neuron was assessed by averaging the gene importances of the associated gene set. Here, gene sets were defined by mapping individual genes to the Reactome pathway collection (C2:CP collection v7.5, downloaded from https://data.broadinstitute.org/gsea-msigdb/msigdb/release/7.5/).

A cluster analysis was performed by visualizing the latent space embeddings on a 2D and 3D space by calculating t-SNE and UMAP projections using their respective python packages.

DNAm imAGE

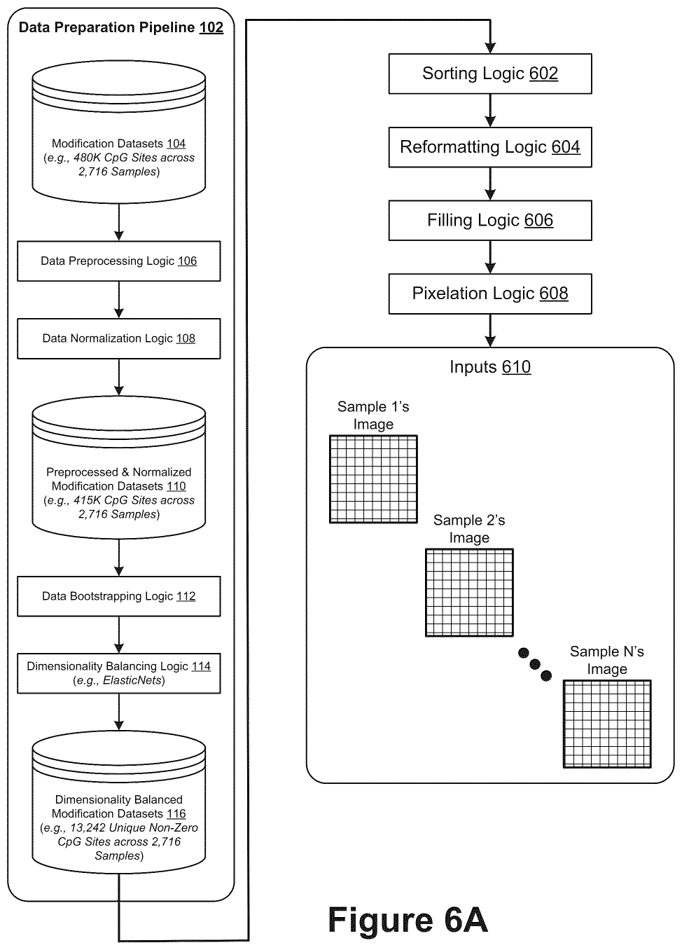

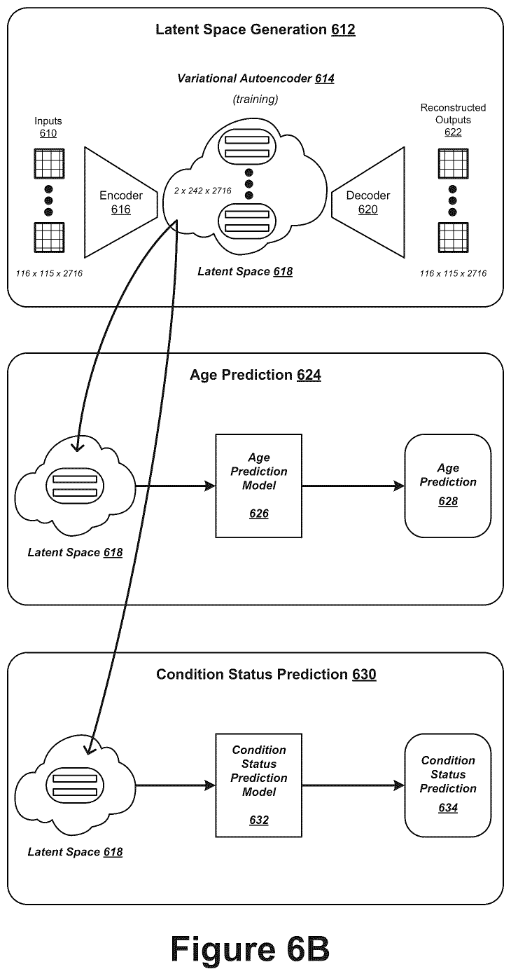

FIG. 6A shows another implementation of the data preparation pipeline 102, along with components 602, 604, 606, 608, and 610.

FIG. 6B shows one implementation of latent space generation 612, comprising components 610, 614, 616, 618, 620, and 622.

FIG. 6B shows one implementation of age prediction 624, comprising components 618, 626, and 628.

FIG. 6B shows one implementation of condition status prediction 630, comprising components 618, 632, and 634.

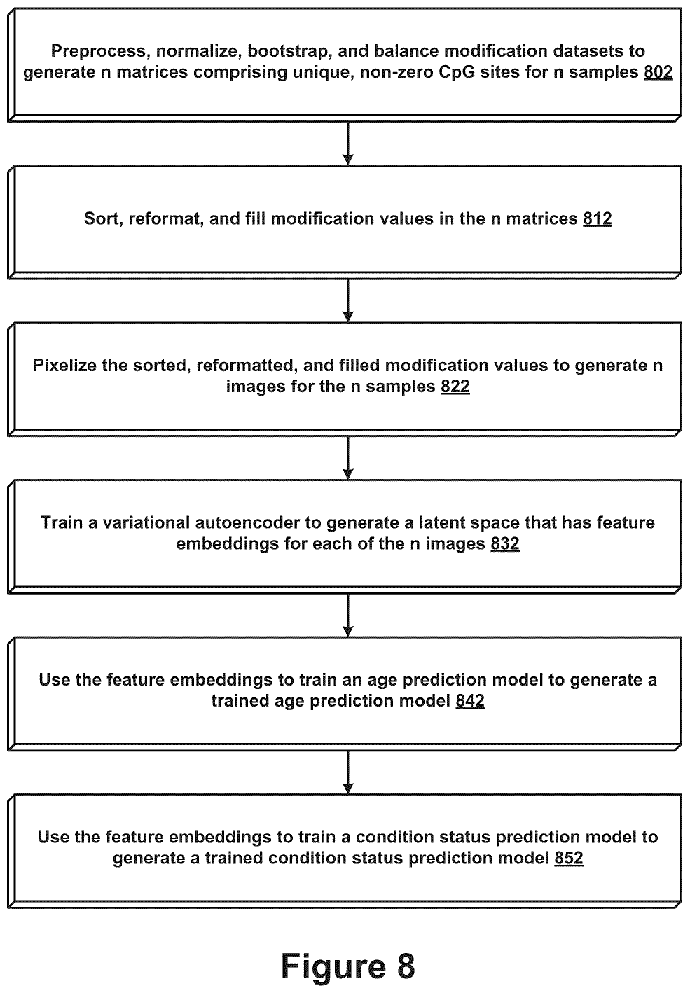

FIG. 8 is a flowchart showing another method implementation of the technology disclosed, including steps 802, 812, 822, 832, 842, and 852.

### Methylation Array Processing

Methylation data used in this study consisted of 2,716 blood samples measured on Illumina's Infinium HumanMethylation450 BeadChip (450K) array originating from seven public datasets. For each CpG site, methylation values are indicated by the beta-value which ranges from 0 (no methylation) to 1 (fully methylated).

Raw beta-values were processed in R (v4.1.2) with the RnBeads package (v2.12.2). Probes not in CpG context were filtered out as well as probes for which the beta-values were NA or had low variability (standard deviation<0.005). Beta values of the remaining probes were next normalized using the Beta Mixture Quantile dilation method (BMIQ) with an Exponential-Normal mixture (Enmix) signal intensity background correction. After preprocessing and normalization, only probes present in all eight datasets were kept resulting in a final dataset of 2,716 samples and 415,594 CpG probes.

Dimensionality Reduction with ElasticNets

Typically, methylation arrays inherently present a dimensionality imbalance (i.e., number of CpGs (p)>>number of samples (n)). To handle the dimensionality imbalance in our dataset (p=415,594, n=2,716), a prior feature selection step was performed using ElasticNets, a shrinkage technique that uses a weighted combination of L1 (Ridge) and L2 (Lasso) regularization.

Before fitting an ElasticNet, samples were bootstrapped with a fraction of 42% and sample weights equal to the respective age class weight to generate a balanced dataset.

This bootstrapping procedure was performed 42 times, and for each bootstrapped distribution an ElasticNet was fitted with an L1/L2 ratio of 0.5 and the non-zero coefficients stored. On average, each ElasticNet had 895 non-zero coefficients with a range of [821; 955]. In a next step, the non-zero coefficients from all bootstrap iterations were combined, resulting in 13,242 unique non-zero coefficients, i.e., CpGs, out of the initial 415,594 (=3.19%).

Converting Numeric Beta Values in Tabular Format to imAGE

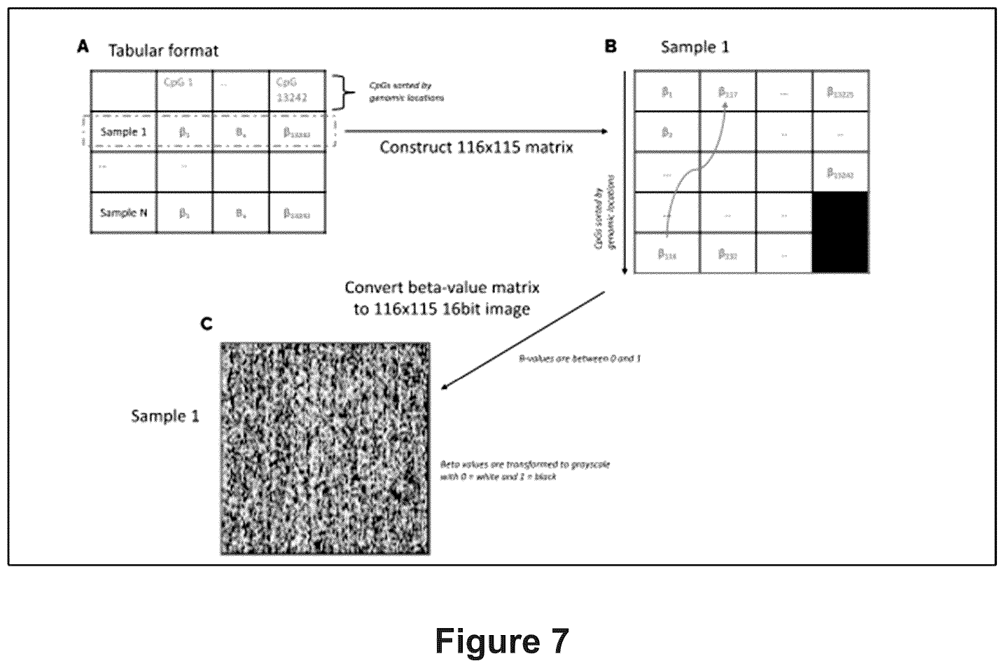

After preprocessing the beta values as described above, the methylation data is contained in a M×N tabular format with M=2,716 samples (rows) and N=13,242 CpGs (columns). The columns in this data matrix are sorted according to the genomic locations of the corresponding CpGs (FIG. 7A). In a next step, the beta-values of the 13,242 CpGs are reformatted to a 116×115 beta-matrix for each sample. Here, the matrix is filled up with beta-values per column according to the CpG location (FIG. 7B). Note that as this matrix contains 13,340 cells, the last 98 cells are left bank. Finally, this beta-value matrix is converted to a 16 bit image (imAGE) where the color of each cell corresponds to the respective beta value. As a 16-bit image can hold over 65,000 shades of grey, the resolutions of the beta-values are encoded to this scale, with a minimum beta value of 0 encoded as white, and the maximum value of 1 is encoded as black (FIG. 7C).

### Variational Autoencoder Model

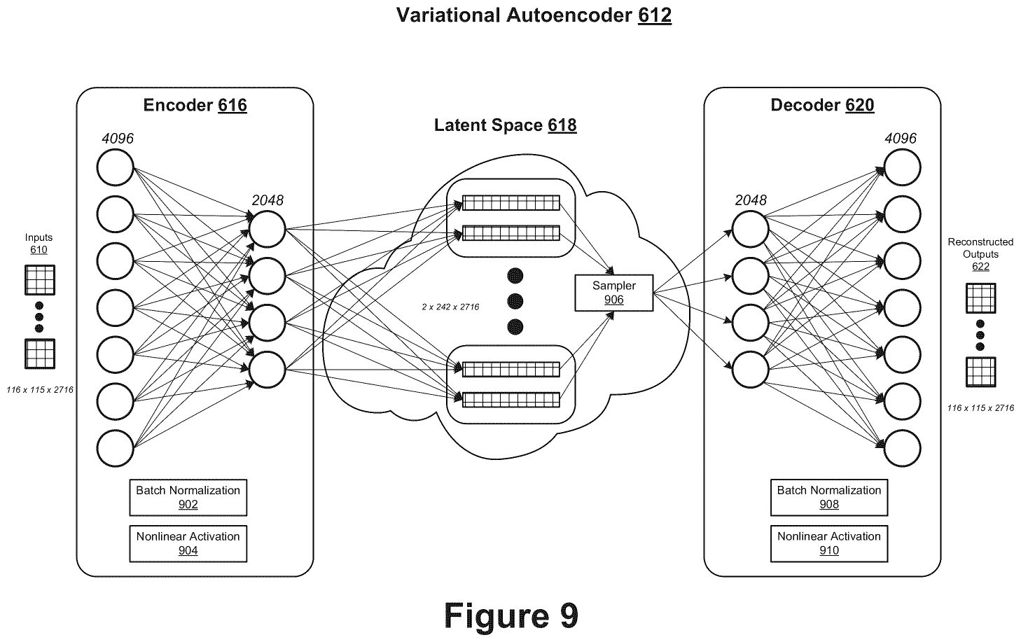

A variational autoencoder (VAE) was developed where the encoder part converts the 2,716 input images to a lower dimensional latent space consisting of two feature vectors representing the input as a jointly Gaussian distribution (FIG. 9) in conjunction with components 902, 904, 906, 908, and 910. The VAE model was trained using the ADAM optimizer with two loss functions: (i) a Mean Squared Error (MSE) loss function capturing the reconstruction loss and (ii) a Kullback-Leibler divergence (KLD) loss function measuring the distance between two distributions. Two KLD implementations were tested: (i) one that determines the KLD between the latent distribution and a Gaussian distribution, thereby assuming hence forcing the sampling in the representation layer to approximate a normal random variable, and (ii) a Monte Carlo approximation of KLD which is distribution agnostic, i.e., without making any prior assumptions about the underlying distribution. The total training loss was defined as: MSEloss+w*KLDloss with w the weight of the KLD term. Here, the weight w varied between 0-1 following a cyclical sigmoid annealing scheduling to mitigate KLD-vanishing.

Samples were shuffled in a training and test set at an 80/20 ratio with stratification on age class. This test set was left out during model training and only used for final model performance. A 10-fold cross validation (CV) performed on the training set. Hyperparameters were empirically determined. Each CV model was trained for 100 epochs using a batch size of 64, a learning rate of 1e-3. To prevent exploding gradients during VAE training, a weight decay (L2 regularization) of 1e-4 was set and gradients were clipped with a norm of 0.01. For each CV, the weights were saved for the epoch with the lowest loss on the validation set. The best model was chosen based on the CV configuration with the highest validation accuracy. This final model was next evaluated on the test set.

### Multi-Laver Perceptron

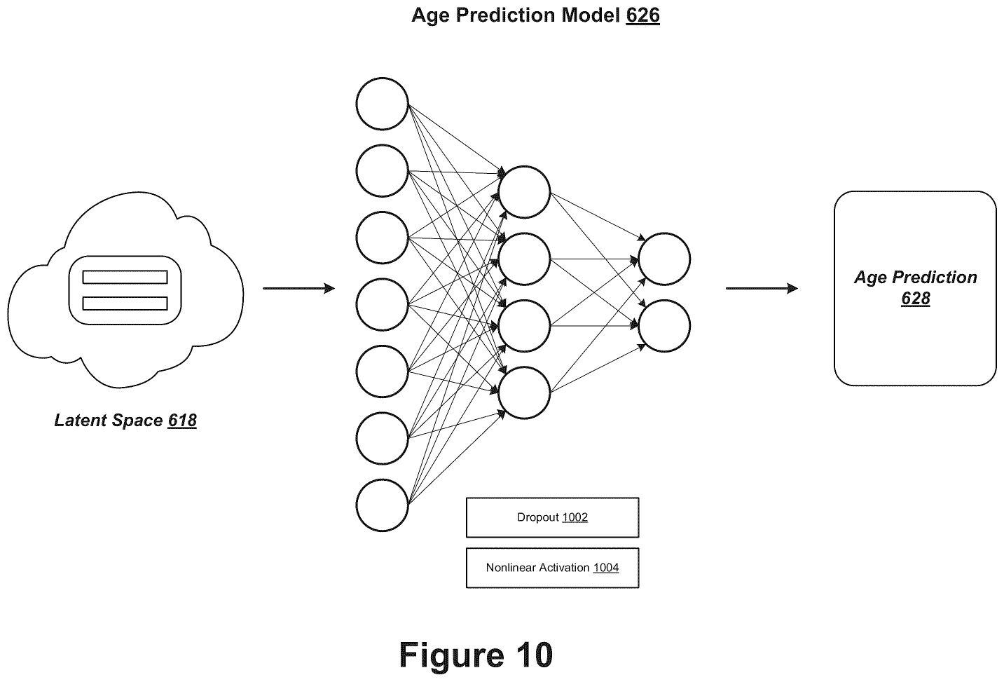

In order to test the utility of the learned latent methylation space to predict age, a three-layer multi-layer perceptron was trained with input layer of size 242 (=size of latent space), 3 hidden layers of size 128, 64, and 32 and output layer of size 1 (FIG. 10), in conjunction with components 1002 and 1004. Before each layer, a dropout with probability 0.42 was implemented and the non-linear ReLu function was used as activation function. As for the VAE, the ADAM optimizer was used together with a weighted MSE loss function, where the error on each sample was weighted according to its age class weight (Table 2). The MLP was trained for 100 epochs, using a batch size of 32, learning rate of 1e-3 and weight decay of 1e-4. Also here, the best model was selected as the epoch with the highest performance on the validation set and evaluated on the test set.

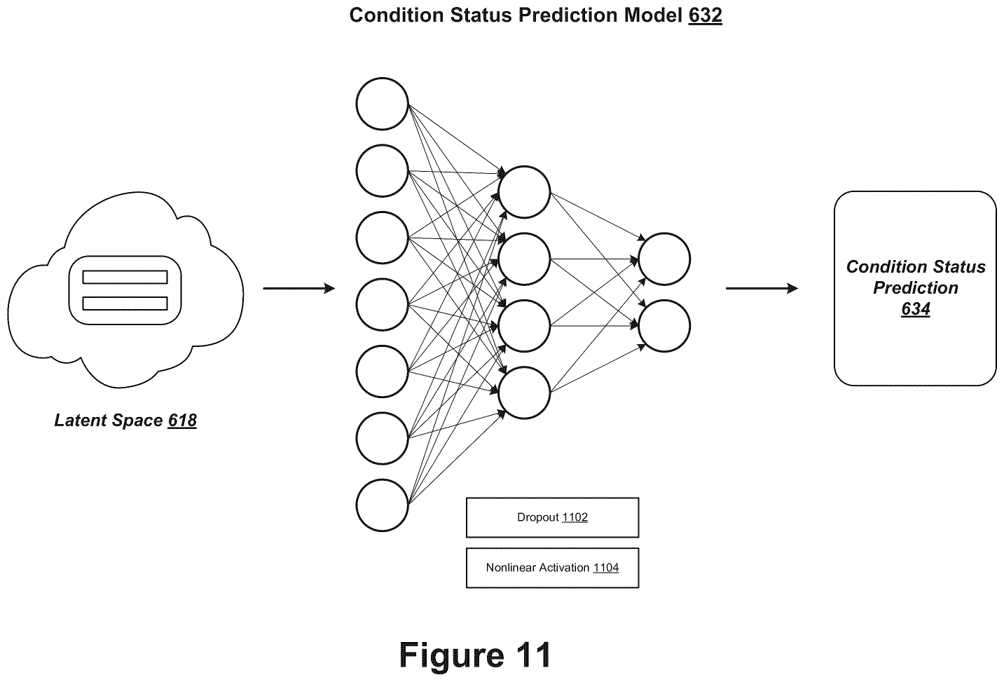

FIG. 11 illustrates another implementation of the disclosed logistic regression model for binary classification to predict rheumatoid arthritis (RA) status from the VAE embedding, comprising components 1102 and 1104.

### Results

### Model Development for Epigenetic Age Prediction

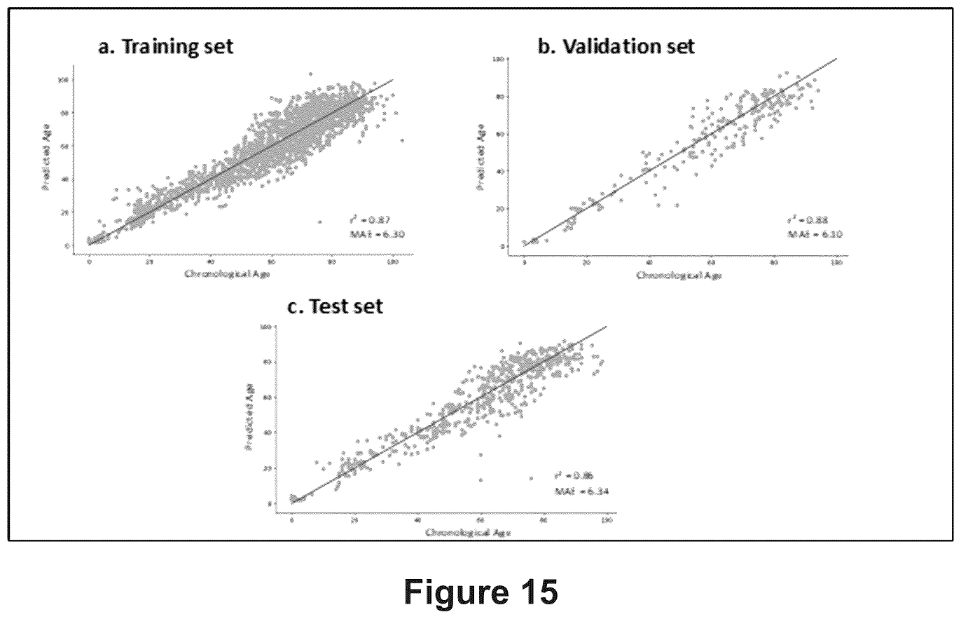

Two VAE frameworks were developed and evaluated for subsequent epigenetic age prediction: (i) a VAE where the KLD is calculated between the latent distribution and a Gaussian distribution, and (ii) a VAE where the KLD is distribution agnostic. For each framework, an MLP was trained using 10-fold cross validation (CV) to predict age using the learned latent spaces of the samples as input (see Methods FIG. 15). Table 3 below shows the CV model performances using two standard evaluation metrics: (i) Mean Absolute Error (MAE) and (ii) the coefficient of determination, r-squared (r2). The first column shows the mean score of the 10-fold CV with the standard deviation for the Gaussian framework on the training, validation and test sets, while the second column shows those performances for the agnostic framework. It can be seen that for all datasets, the agnostic framework performs better (both lower MAE and higher r2). A pairwise comparison (Wilcoxon signed-rank test) of both the MAE and r2 distributions indeed revealed significantly improved performance of the agnostic framework over the Gaussian one (FIG. 15).

FIG. 15 displays the corresponding predictions and evaluation metrics of the best-performing CV agnostic model for all samples of the training, validation sets, and test sets. Here, the x-axis shows the chronological age of each sample, while the y-axis represents the age predicted by the model. The final model, which we named methVAgE, reached an MAE of 6.34 and an r2 on the test set.

TABLE 3

KLD strategy

| Dataset | Metric | Gaussian | Agnostic |
|---|---|---|---|
| Training | Mean(MAE) CV ± | 7.00 ± | 6.35 ± |
| | stdev | 0.23 | 0.25 |
| | Mean(r²) CV ± | 0.84 ± | 0.87 ± |
| | stdev | 0.01 | 0.01 |
| Validation | Mean(MAE) CV ± | 6.83 ± | 6.20 ± |
| | stdev | 0.43 | 0.38 |
| | Mean(r²) CV ± | 0.85 ± | 0.87 ± |
| | stdev | 0.01 | 0.02 |
| Test | Mean(r²) CV ± | 7.22 ± | 6.54 ± |
| | stdev | 0.31 | 0.28 |
| | Mean(r²) CV ± | 0.83 ± | 0.85 ± |
| | stdev | 0.01 | 0.01 |

### Model Evaluation on External Datasets

After pre-processing the methylation data of the GENOA (African American sibling cohort from the Genetic Epidemiology Network of Arteriopathy (GENOA)) and rheumatoid arthritis (RA) datasets, the developed model was next validated on these two external cohorts.

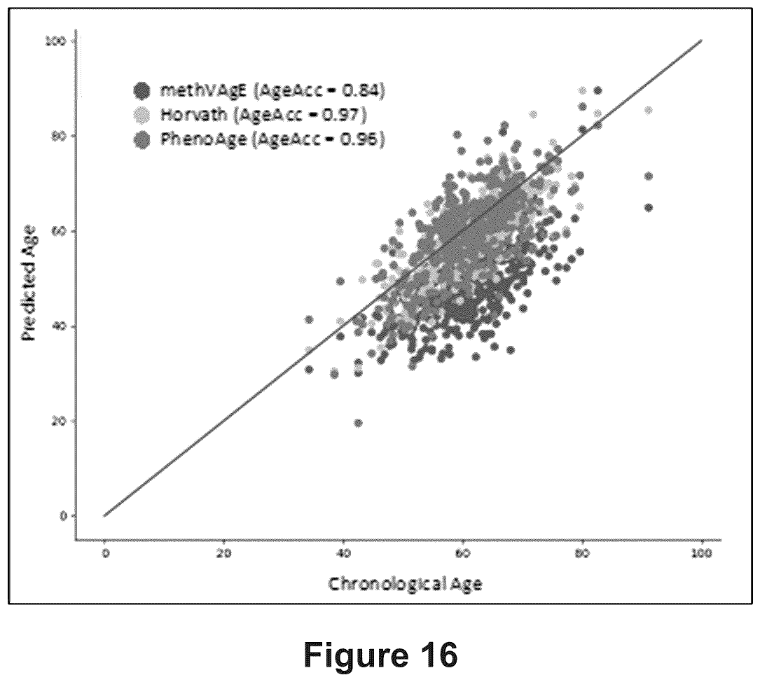

FIG. 16 shows the predictions on the 418 GENOA samples for three epigenetic clocks: (i) our VAE+MLP framework (methVAgE), and two other well-known linear model clocks, (ii) Horvath and (iii) PhenoAge. As for FIG. 16, the x-axis shows the chronological age of each sample, while the y-axis represents the predicted DNAm age. In addition to the model predictions, we also calculated the age acceleration (AgeAcc) of each sample as the predicted DNAm age divided by the chronological age. Here, an AgeAcc higher than 1 corresponds with accelerated biological ageing, while a value lower than 1 corresponds to decelerated biological ageing (which is preferred). FIG. 16 displays the mean AgeAcc of all samples.

Note how the DNAm predictions of our model are on cohort-level lower than the corresponding chronological ages, which is also reflected in a lower AgeAcc compared to the other two clocks. Interestingly, it has already been shown that African Americans indeed have a significantly lower extrinsic DNAm age compared to whites. And while Horvath and PhenoAge also have an average AgeAcc<1 for this cohort, it is less pronounced, hinting that our framework might better capture underlying biological signals. We also calculated the AgeAcc for a third well-known epigenetic clock, i.e. Hannum. This clock has an average AgeAcc of 1.06, meaning that this biological phenomenon is not captured by this clock.

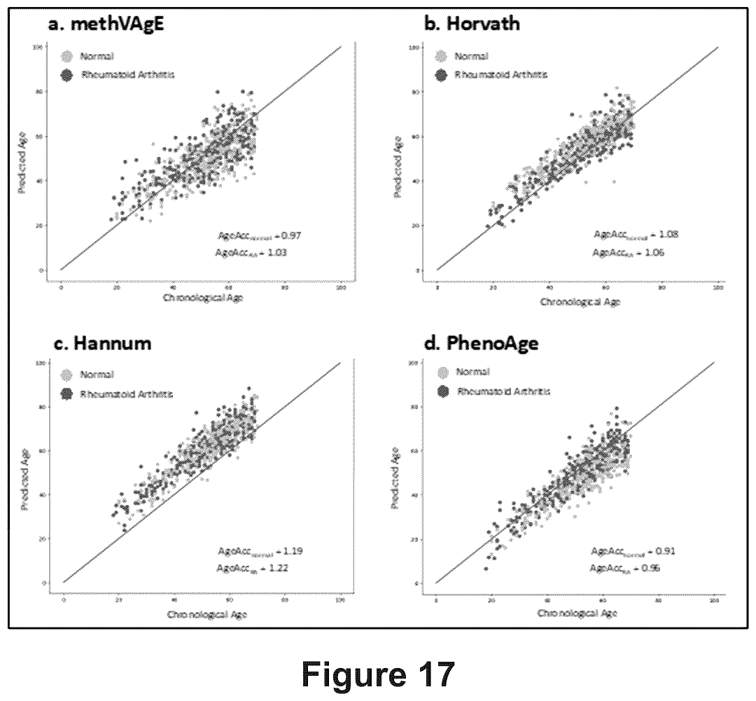

FIG. 17 shows the model predictions of different clocks for the second cohort, i.e. the RA dataset consisting of 689 samples, of which 335 (49%) are healthy control samples and 354 (51%) have been diagnosed with RA. While the researchers of this study found a significant age difference between these two groups (p=0.0049, although with small effect size, i.e. 1.2 years), Horvath did not find a significant difference in negative age acceleration between diseased versus healthy samples in his study. This can also be seen in FIG. 17, where we computed the DNAm age predictions and the subsequent mean AgeAcc using Horvath's algorithm. There is indeed no significant difference in AgeAcc, and in fact the AgeAcc is even marginally higher for the healthy cohort. However, for the other three algorithms, the diseased samples indeed show an increased AgeAcc, with a small difference when using Hannum's clock (0.3), and a more pronounced difference in AgeAcc of 0.5 and 0.6 for PhenoAge and our methVAgE model, respectively. Note that compared to PhenoAge, our model results in an average cohort AgeAcc>1 for the RA samples, indeed indicating that on average, the RA subgroup age faster than normal.

In addition to calculating AgeAcc, we also investigated if the latent space encoded by our VAE contains biological signals that can be used as input of a classifier to predict (age-related) diseases.

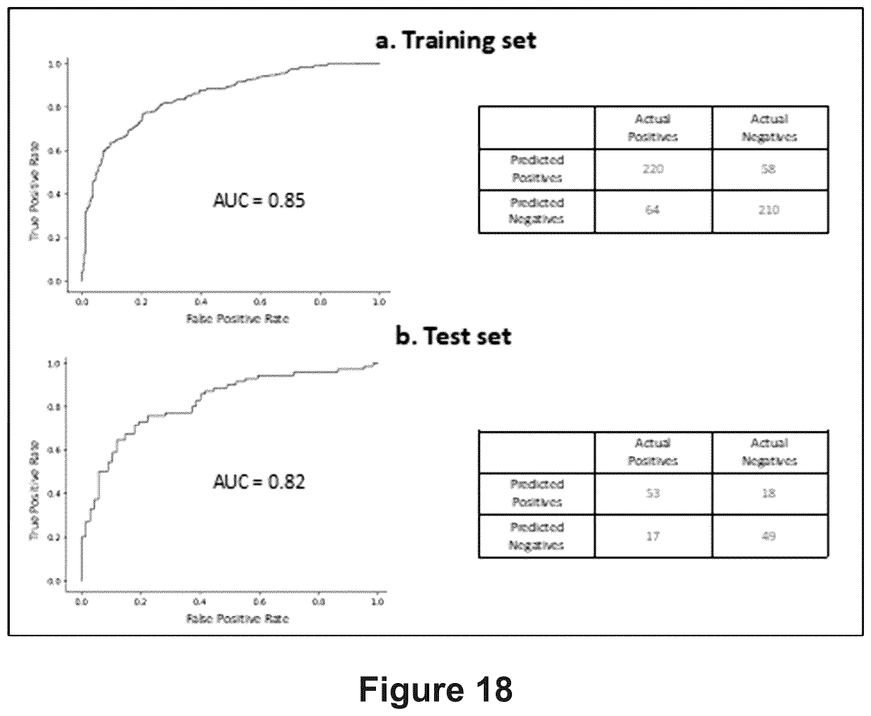

Here, the idea is that the encoded feature space is potentially different between healthy and diseased samples and that a model can be trained to leverage these differences and classify a sample as diseased or healthy. To test this hypothesis on this RA dataset, we trained a simple binary logistic regression model to predict RA status using the VAE embeddings as input. FIG. 18 shows the performance of this model on the training and the test set. The left panel shows the ROC curves with the respective AUC score, while the right panel shows the confusion matrix. The results accomplished with this simple model illustrate that the VAE embeddings indeed contain encoded biological signals that can potentially be leveraged to distinguish diseased from healthy samples.

### Model Interpretation

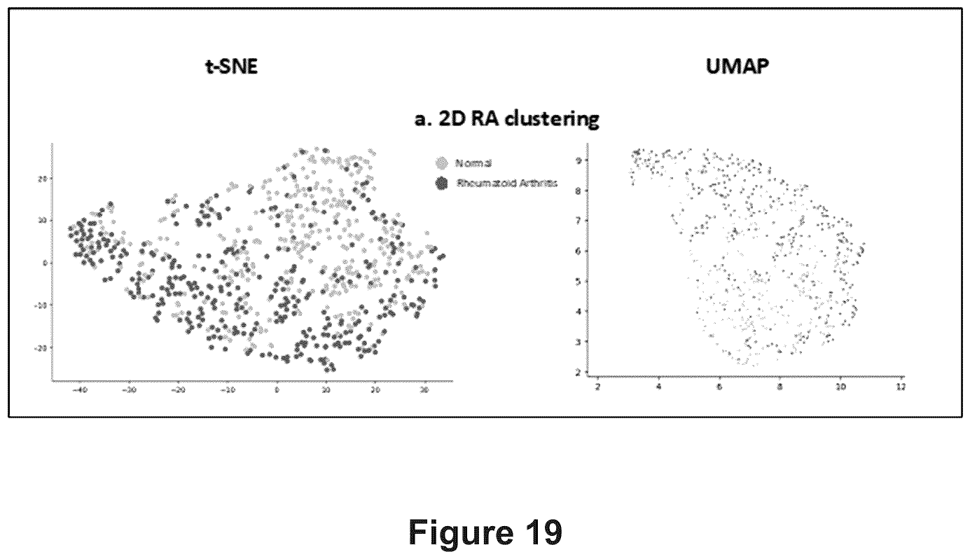

Given the results above, we subsequently performed an unsupervised cluster analysis to check if given only the VAE embeddings as input, if such an analysis independently also reveals a difference between the two groups. FIG. 19 shows the 2D presentation of two different unsupervised cluster methods, i.e. t-SNE and UMAP. It can be seen that both methods cluster the majority of the RA embeddings together in one group, while the normal samples are clustered in in another group. This independently shows that the VAE embeddings in the two groups indeed possess different signals which are potentially biologically relevant to RA.

One way to unravel which biological signals are encoded in the VAE latent space is via a pathway analysis of the model gradients. This can be done per sample, but also per group to compare how which pathways are different between group. Here, we did this for RA versus normal samples. by backpropagating the VAE model and calculating the gradients for each gene in the embedding. Individual genes were next grouped into Reactome pathways and average pathway gradients were calculated. In a next step, these pathway gradients were compared between the two groups.

Table 4 lists some of the top pathways that were found to be notable different between the RA and healthy group. The first column shows the pathway name, while the second column shows the ratio of the pathway importance in the RA group and the importance of that pathway in the normal group. Column three list some references on the involvement of each pathway in RA. For example, the first pathway (MAPK pathway) has a ratio of 10.91 meaning that the presence of this pathway in the RA embeddings is almost 11 times higher than in the embeddings of the normal group. Interestingly, this pathway has indeed already been shown to be important in RA, and is implicated in many processes that underly the pathology of RA (see references in third column). On the other spectrum, the last pathway in this table (activation of the AP1 family of transcription factors) is almost not represented in the RA group while it is prominent in the normal group. Previous studies indeed have shown that AP-1 activation is needed for DNA synthesis, and that during ageing and in age-related diseases, the activity of AP-1 is indeed lost. Hence illustrating the low presence of this pathway in the RA group.

TABLE 4

| | RA/normal |
|---|---|
| | importance |
| Pathway | ratio |
| NEGATIVE FEEDBACK REGULATION OF MAPK PATHWAY | 10.91 |
| P75NTR NEGATIVELY REGULATES CELL CYCLE VIA SC1 | 2.00 |
| NF KB IS ACTIVATED AND SIGNALS SURVIVAL | 1.59 |
| N GLYCAN TRIMMING AND ELONGATION IN THE CIS GOLGI | 1.52 |
| FGFR3 LIGAND BINDING AND ACTIVATION | 1.5 |
| TNFR1 MEDIATED CERAMIDE PRODUCTION | 0.6 |
| SIGNALING BY MST1 | 0.53 |
| COMPETING ENDOGENOUS RNAS CERNAS REGULATE PTEN | 0.53 |
| TRANSLATION | |
| SYNTHESIS OF GDP MANNOSE | 0.005 |
| ACTIVATION OF THE AP1 FAMILY OF TRANSCRIPTION | 0.001 |
| FACTORS | |

This analysis shows that the encoded feature space of the two groups is different, and that it is possible to find biologically explain these differences. Although unsupervised, the VAE is able to distinguish between these two groups and creates a biologically relevant latent space that can be used to extract relevant biological signals related to ageing and/or age-related disease, but also to model DNAm clocks to predict biological age.

In conclusion, all the above results showcase the strength of our framework and the ability to capture underlying biological signals. Because of the latter, our framework provides a better proxy for “true” biological age compared to other epigenetic clocks, which are optimized for chronological age.

### Methylation Differences in Rheumatoid Arthritis (RA) Patients Compared to Normal Controls Using Machine Learning

In this disclosure, we aimed to investigate whether rheumatoid arthritis (RA) patients exhibit methylation differences compared to normal controls. We utilized a dataset (GEO ID GSE42861) containing 689 peripheral blood leukocytes samples from a DNAm study. The dataset consisted of 450K methylation array data and was employed to develop a machine learning-based approach for identifying CpGs associated with RA.

### Methods

### 1. Dataset Selection and Preprocessing:

We obtained the dataset (GEO ID GSE42861) comprising peripheral blood leukocytes samples for our analysis. The dataset was comprised of 689 samples and utilized the 450K methylation array data.

2. CpG Selection using Elastic Net:

We employed machine learning techniques to select the CpGs significantly associated with RA. The dataset was divided into training and test sets in an 80/20 ratio. We employed a generalized linear model called Elastic Net, which was trained on the training set using 10-fold cross-validation. This Elastic Net model was utilized as a preliminary feature selection step to address the issue of dimensionality. Importantly, only CpGs within CpG islands and associated with promoter regions were used for training the ENET model, this because these CpGs can be potentially used to develop a methyl-specific PCR at a later stage for disease prediction.

For model training, feature scaling was applied, and a 10-fold cross-validation strategy was used, along with random resampling (with replacement), to achieve a more uniform distribution of the age population. The CpGs were subsequently ranked based on their model importance, and a final selection of 16 CpGs was retained.

### 3. Logistic Regression Classification Model:

In the next step, a logistic regression classifier model was trained on the training set using the 16 selected CpGs to predict the RA disease status. The performance of the model was evaluated using various metrics on both the training and test sets.

### Results

The performance of the logistic regression classifier model was assessed using the following metrics on both the training and test sets:

### On the Training Set:

Area Under the Curve (AUC): 0.925
F1 Score: 0.83
Sensitivity: 0.84
Specificity: 0.82
Positive Predictive Value: 0.84
Negative Predictive Value: 0.83

### On the Test Set:

Area Under the Curve (AUC): 0.890
F1 Score: 0.83
Sensitivity: 0.83
Specificity: 0.84
Positive Predictive Value: 0.84
Negative Predictive Value: 0.82

### Conclusion

In conclusion, the technology disclosed utilized a machine learning-based approach to identify CpGs associated with rheumatoid arthritis. By training a logistic regression classifier model on the selected CpGs, we achieved promising predictive performance on both the training and test sets. These findings suggest that this methodology of only considering CpGs in CpG islands and promoter (associated) regions in a first feature selection after which a disease classifier is trained, is a promising approach to find a suitable set of CpGs that can be used to develop a methyl-specific PCR assay for disease prediction. Importantly, while we showed this here for RA, this methodology can also be used for other diseases of interest.

Detecting Rheumatoid Arthritis with Marker Genes

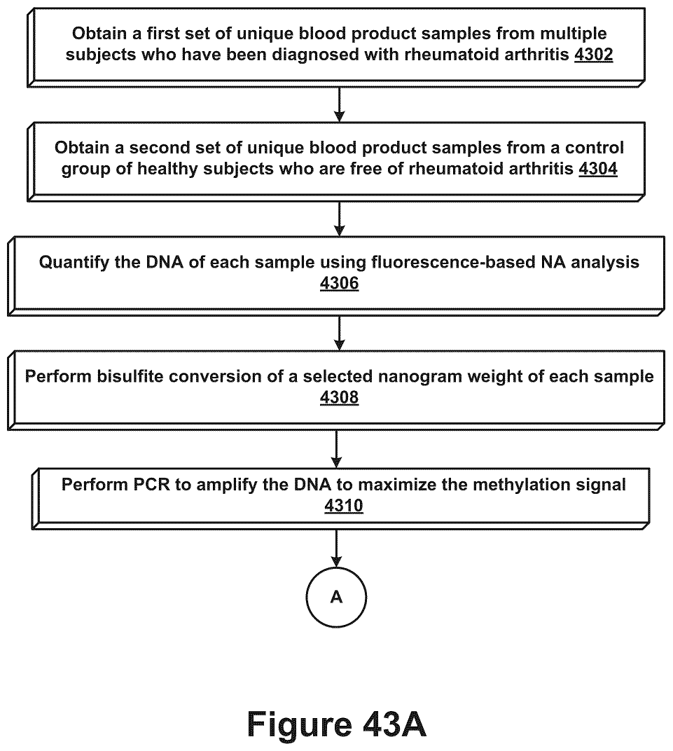

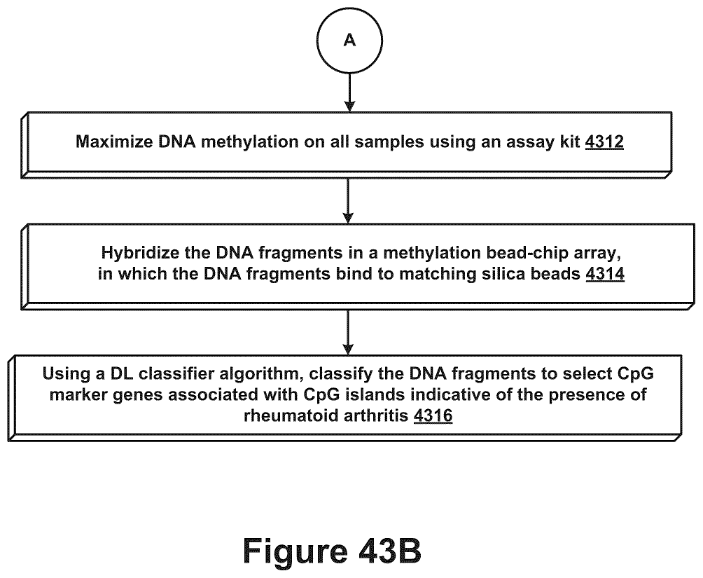

In another aspect of the present technology, a computer-implemented method is disclosed for detecting rheumatoid arthritis in patients using marker genes. Blood leukocyte samples provided by patients are used to determine whether rheumatoid arthritis (RA) patients have methylation differences comparing to normal controls. The computer-implemented method is illustrated in FIGS. 43A and 43B and in the following text. Turning to FIGS. 43A and 43B, an overview illustration of the present technology is shown.

In step 4302, a first set of unique blood products samples are collected from multiple subjects who have been diagnosed with rheumatoid arthritis. In step 4304, a second set of unique blood products samples are collected from a control group of healthy subjects who are free from rheumatoid arthritis.

The samples are processed as follows. In step 4306, the DNA of each sample is quantified using fluorescence-based nucleus acid analysis. In the next step, 4308, a by sulfite conversion is performed on a selected nanogram weight of each sample. In step 4310, a polymerase chain reaction (PCR) is performed to amplify the DNA and simultaneously maximize the methylation signal.

In step 4312, DNA methylation on all samples is maximized using an assay kit. DNA fragments are hybridized in step 4314 in a methylation bead-chip array, in which the DNA fragments are bound to matching silica beads.

In step 4316, using a deep learning classifier algorithm, the DNA fragments are classified select CpG marker genes associated with CpG islands which have been shown to indicate the presence of rheumatoid arthritis.

In one aspect, the present technology uses a machine-learning based approach to identify CpGs associated with rheumatoid arthritis. By training a logistic regression classifier model on selected CpGs, promising predictive performance on both the training and test sets is achieved. A computer-implemented methodology of considering only CpGs in CpG islands and promoter (associated) regions in a first feature selection, after which a disease classifier is trained, is a promising approach to find a suitable set of CpGs that can be used to develop a methyl-specific PCR assay for disease prediction. While the results here are shown for rheumatoid arthritis, this computer-implemented methodology may also be used for other diseases of interest.

Table 5 shows 16 major genes indicative of rheumatoid arthritis and their associated CpG data.

Three marker genes are of particular interest in identifying rheumatoid arthritis. These are shown in Table 6. These marker genes are identified as RCAN3, HDAC4, and SIPA1, as shown in Table 6, with their forward primer and reverse primer gene sequences.

TABLE 6
The Primer Sequences of The Selected Marker Genes

| | UCSC | Illumina | | | | | |
|---|---|---|---|---|---|---|---|
| | RefGene | 850K EPIC | | | | | Amplicon |
| | accession | Methayl- | | | | Sequence | length |
| Gene | number | ation | Position | Assay | | (5′ to 3′) | (hp) |
| RCAN3 | NM_013441 | cg16411857 | chr1: | RA_301 | Forward | GTTGGAAGTGATGT | 139 |
| | | | 24535382-24535383 | | Primer | TTTTGTTATGGTTA | |
| | | | | | Reverse | TTATCTCTTCTTCC | |
| | | | | | Primer | TCTTCAATTTCAC | |
| | | | | RA_302 | Forward | GTGAAATTGAAGAG | 136 |
| | | | | | Primer | GAAGAAGAGATAA | |
| | | | | | Reverse | CACCACAACCAAAA | |
| | | | | | Primer | ACCTCACAA | |
| HDAC4 | NM_006037 | 0005870586 | chr2: | RA_1101 | Forward | GTTAGGGATAGTTT | 154 |
| | | | 239275073-239275074 | | Primer | TAGGAGGTTAG | |
| | | | | | Reverse | CTATCATTACCATC | |
| | | | | | Primer | TCACCCATC | |
| | | | | RA_1102 | Forward | GATGGGTGAGATGG | 178 |
| | | | | | Primer | TAATGATAG | |
| | | | | | Reverse | ACAAATAATCTTTA | |
| | | | | | Primer | CAAAAAACCCTATG | |
| SIPA1 | NM_153253, | cg09762242 | chr11: | RA_1801 | Forward | AGGGTAGGAATTGT | 106 |
| | NM_006747 | | 65640958-85840959 | | Primer | TGTTATAATTTTA | |
| | | | | | Reverse | CCCACATAAACATA | |
| | | | | | Primer | CTCTATAAACC | |

The performance of these three marker genes in identifying rheumatoid arthritis in patients are shown in Tables 7-9 in the following. The probability associated with positive and negative rheumatoid arthritis is also shown each marker gene.

In one aspect of the present technology, a computer-implemented method is disclosed for forming marker genes for predicting the likelihood of a patient having rheumatoid arthritis, or contracting rheumatoid, or being diagnosed as having rheumatoid arthritis (RA). In this computer-implemented method, a unique set of blood products samples are collected from multiple subjects who have been diagnosed with rheumatoid arthritis. The second set of unique blood products samples is collected from multiple subjects who have been diagnosed with rheumatoid arthritis. The DNA of the blood products samples are quantified using fluorescence-based nucleic acid analysis. A bisulfite conversion a selected nanogram weight of each sample is performed. The polymerase chain reaction is used to amplify the DNA to maximize the methylation signal. The DNA methylation is maximized using an assay kit that preserves methylated sites while non-methylated locations are replaced.

The DNA fragments are hybridized in a bead-array boot in which the DNA fragments bind to matching silica. Finally using a deep learning classifier algorithm, the DNA fragments are classified to select CpG marker genes associated with CpG islands indicative of the presence of rheumatoid arthritis. In another aspect, flanking parameters are used to amplify flanking primers used to amplify the region of DNA containing CpG for identifying rheumatoid arthritis sites.

Using this computer-implemented method, marker genes having high significance in identifying rheumatoid arthritis sites have been identified. Three marker genes have been identified as a noteworthy in distinguishing rheumatoid patients from healthy control patients. The marker genes are RCNA3, HDAC4, and SIPA1. In another aspect, the classifier algorithm classifies genomic blood product data as positive for rheumatoid arthritis or negative for rheumatoid arthritis based on the presence of selected marker genes in the blood product sample. In a further aspect, a blood product sample associated with a new patient is assayed to identify if any selected marker genes are present in the blood product sample. Still further, a patient's blood product sample is assayed to determine the presence of the marker genes: RCNA3, HDAC4, and SIPA1. In another embodiment, selected marker genes are formed for predicting diseases other than or in addition to rheumatoid arthritis in a patient.

The computer-implemented method includes assaying the DNA segments of blood product samples to detect the RCAN3 gene for distinguishing rheumatoid patients from control patents. The computer-implemented method also includes assaying the DNA segments of blood products samples to detect the HDAC3 gene for distinguishing rheumatoid patients from healthy control patents. The computer-implemented method further includes assaying the DNA segments from blood products samples to detect the SIPA1 gene for distinguishing rheumatoid patients from healthy control patients. In another aspect, the computer-implemented method includes assaying a patient's blood products to identify a marker gene selected from a group of marker genes consisting of RCAN3, HDAC4, and SIPA1 for distinguishing rheumatoid patients from healthy control patients. In another aspect, a RCAN3 marker gene is formed for distinguishing rheumatoid patents from healthy control patients. In another aspect a HDAC4 marker gene is formed for distinguishing rheumatoid patents from healthy control patient. In still another aspect, a SIPA1 marker gene is formed for distinguishing rheumatoid patients from healthy control patients.

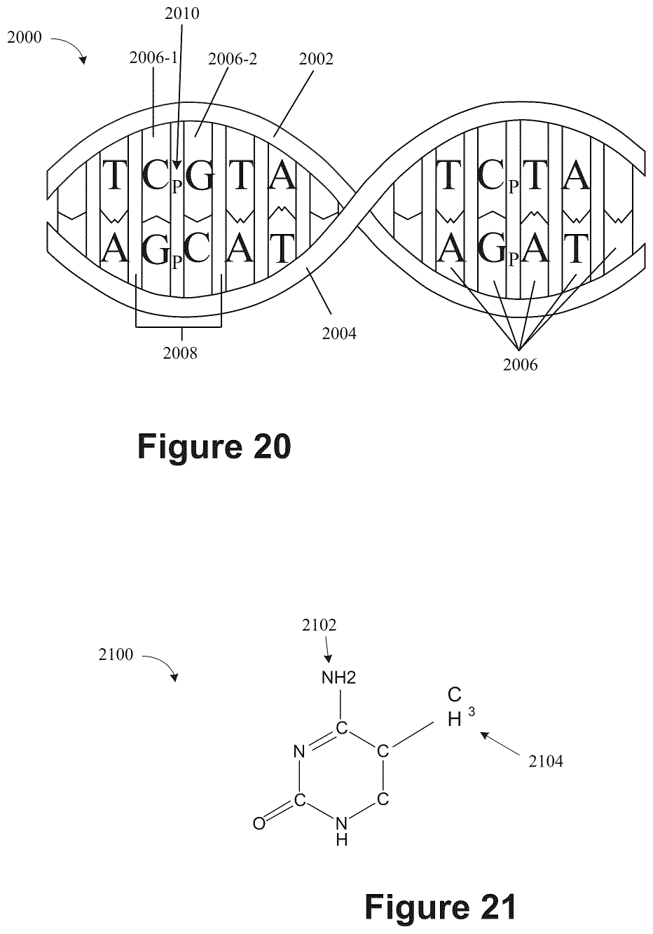

FIG. 20 is a diagram showing an example portion 2000 of DNA. Portion 2000 includes strand 2002 and strand 2004. Nucleotide base pairs 2006 extend along the length of strands 2002 and 2004. An area where a cytosine nucleotide 2006-1 is followed by a guanine nucleotide 2006-2 is called a CpG site 2008. CpG sites 2008 are also defined in that only one phosphate group 2010 separates the cytosine and quinine nucleotide base pairs 2006.

FIG. 21 is a molecular structure diagram showing an example 5-Methylcytosine molecule 2100. 5-Methylcytosine is an example of a methylated form of cytosine. Molecule 2100 includes a cytosine portion 21021 and a methyl group portion 2104. As shown, the methyl group portion 2104 is attached to the 5th atom in the 6-atom ring, counting counterclockwise from the NH-bonded nitrogen at the six o'clock position of the cytosine portion 21021.

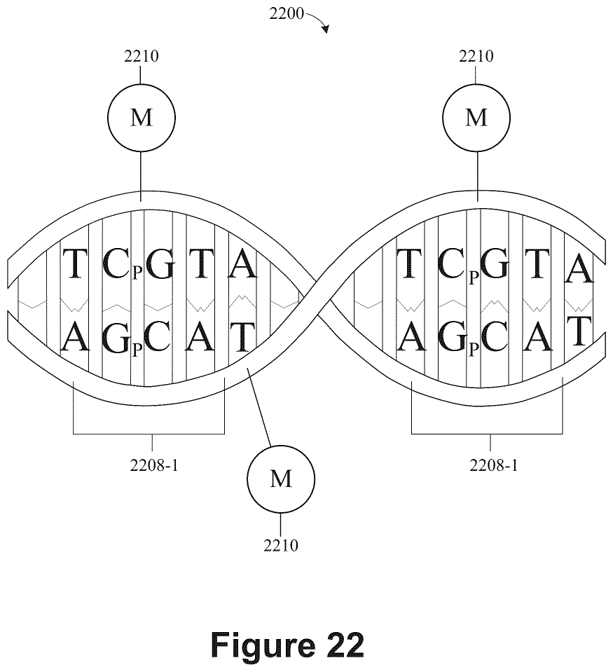

FIG. 22 is a diagram showing an example portion 2200 of DNA. Portion 2200 includes two CpG sites 2208-1 and 2208-2 (collectively referred to as CpG sites 2208). The first CpG site 2208-1 has two methyl groups 2210 attached to the two cytosine molecules. This configuration of CpG site 2208-1 is called being fully methylated. The second CpG site 2208-2 has a single methyl group 2210 attached to one of the two cytosine molecules. This configuration of CpG site 2208-2 is called being hemi-methylated. When there are no methyl groups attached to either cytosine, then the CpG site is considered to be unmethylated.

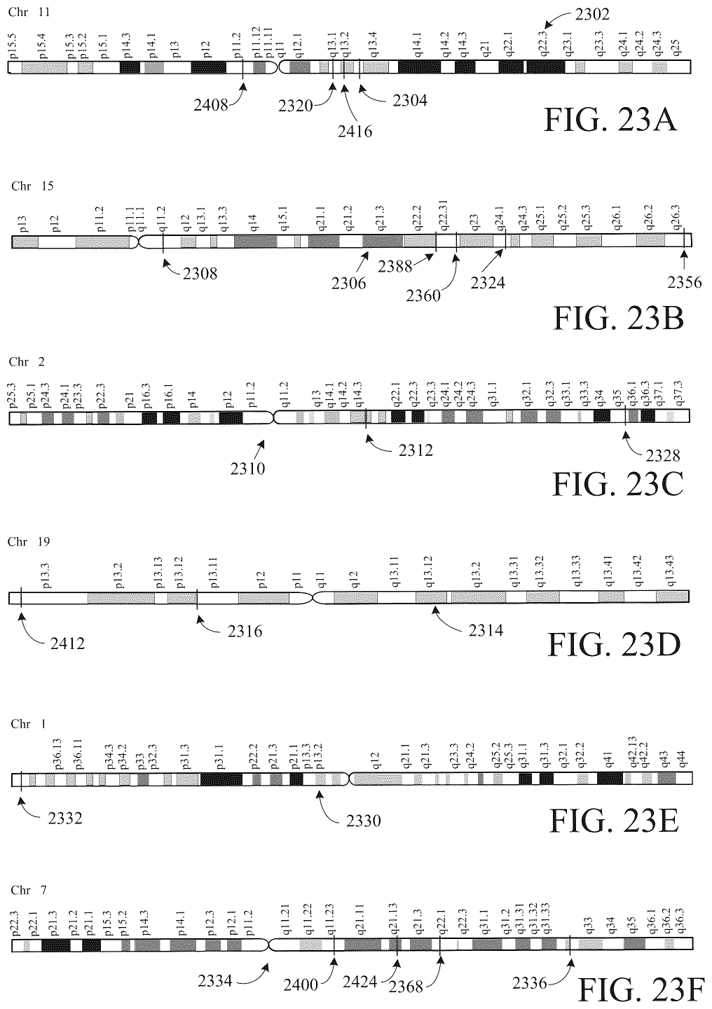

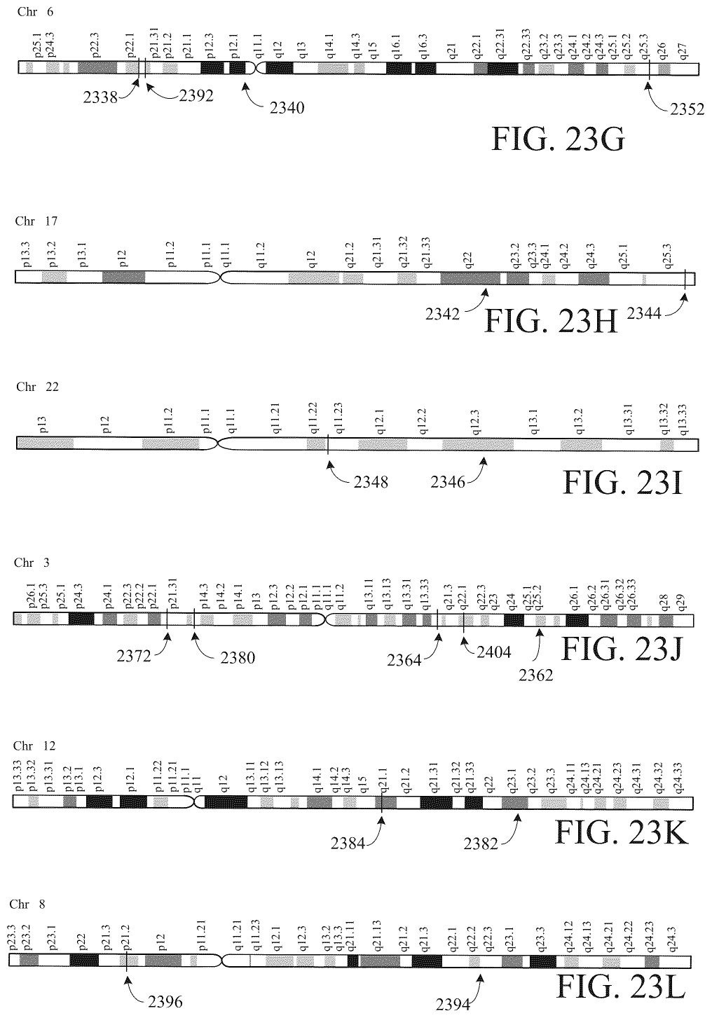

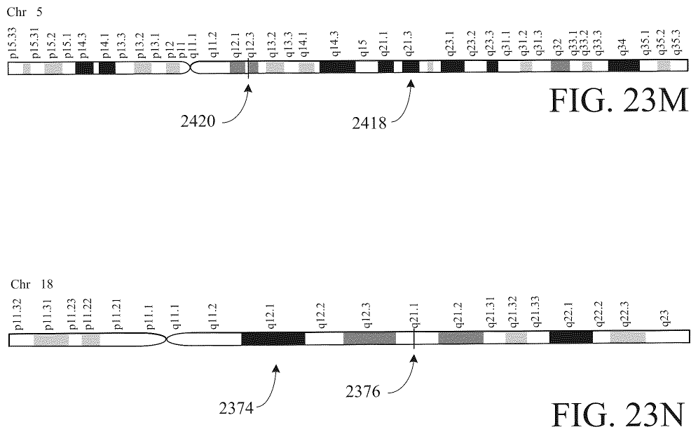

FIG. 23A-N illustrate genomic diagrams of chromosomes. Epigenetics, by definition, can alter the way in which a gene behaves based on non-sequence changing factors like methylation. If key aging gene behaviors are modified by methylation, then the methylation status of CpG sites in these genes may provide insight on the biological age of an individual.

FIG. 23A illustrates a genomic diagram 2302 of chromosome 11. At location 2304 is the Fibroblast growth Factor 19 (FGF19) gene. FGF family members possess broad mitogenic and cell survival activities, and are involved in a variety of biological processes including embryonic development cell growth, morphogenesis, tissue repair, tumor growth and invasion. The FGF19 protein is produced in the gut where it functions as a hormone, regulating bile acid synthesis, with effects on glucose, cholesterol, and lipid metabolism. Reduced synthesis, and blood levels, has been linked to chronic bile acid diarrhea and as well as certain metabolic diseases. FGF19 may be used in treatment of metabolic disease. FGF19 plays a role in maintaining health and metabolic homeostasis. High levels of circulating FGF19 increases energy spending and reduces fat tissue. FGF19 can have hypertrophic effect on skeletal muscle. FGF19, therefore, appears be a therapeutic target to limit aging-associated muscle loss and other diseases characterized by muscle atrophy (obesity, cancer, kidney failure). The CpG site, Cg27330757, is a probe mapping to protein coding Fibroblast growth Factor 19 (FGF19) gene.

At location 2320 is the Mono-ADP Ribosylhydrolase 1 (MACROD1) gene. The protein encoded by this gene has a mono-ADP-ribose hydrolase enzymatic activity, i.e. removing ADP-ribose in proteins bearing a single ADP-ribose moiety, and is thus involved in the finetuning of ADP-ribosylation systems. ADP-ribosylation is a (reversible) post-translational protein modification controlling major cellular and biological processes, including DNA damage repair, cell proliferation and differentiation, metabolism, stress, and immune responses. MACROD1 appears to primarily be a mitochondrial protein and is highly expressed in skeletal muscle (a tissue with high mitochondrial content). It has been shown to play a role estrogen and androgen signaling and sirtuin activity. Dysregulation of MACROD1 has been associated with familial hypercholesterolemia and pathogenesis of several forms of cancer, and particular with progression of hormone-dependent cancers. The CpG site, cg15769472, is a probe mapping to the protein coding MACROD1 gene.

At location 2408 is the Diacylglycerol Kinase Zeta (DGKZ) gene. The DGKZ gene coding for the diacylglycerol kinase zeta. This kinase transforms diacylglycerol (DAG) into phosphatidic acid (PA). The latter product activates mTORC1. The overall effect of mTORC1 activation is upregulation of anabolic pathways. Downregulation of mTORC1 has been shown to drastically increase lifespan. The CpG site, cg00530720, is a probe mapping to the promoter of the DGKZ gene.

At position 2416 is the CD248 Molecule (CD248) gene. The CpG site, cg06419846, is a probe mapping to the CD248 gene.

FIG. 23B illustrates a genomic diagram 2306 of chromosome 15. At location 2308 is the WAS Protein Homolog Associated with Actin, Golgi Membranes and Microtubules Pseudogene 3 (WHAMMP3) gene. This pseudogene has been associated with Prader-Willi syndrome, a severe developmental disorder. This syndrome is caused by epigenetics defect on chromosome 15. More specifically the absence of paternally expressed imprinted genes at 15a11.2-q13, paternal deletions of this region, maternal uniparental dysomy of chromosome 15 or an imprinting defect. Multiple imprinted genes in this region contribute to the complete phenotype of Prader-Willi. The CpG site, cg04777312, is a probe mapping to the pseudogene WHAMMP3.

At location 2324 is the PML gene. The phosphoprotein coded by this gene localizes to nuclear bodies where it functions as a transcription factor and tumor suppressor. Expression is cell-cycle is related and it regulates the p53 response to oncogenic signals. The gene is often involved in the PML-RARA translocation between chromosomes 15 and 17, a key event in acute promyelocytic leukemia (APL). The exact role of PML-nuclear body (PML-NBs) interaction is still under further investigation. Current consensus is that PML-NBs are structures which are involved in processing cell damages and DNA-double strand break repairs. Interestingly, these PML-NBs bodies have been shown to decrease with age and their stress response also declines with age. The latter can be in a p53 dependent or independent way. PML has also been implicated in cellular senescence, particularly its induction and acts as a modulator of the Werner syndrome, a type of progeria. The CpG site, cg05697231, is a probe mapping to the south shore of a CpG island in the PML gene.

At location 2356 is the ADAM Metallopeptidase with Thrombospondin Type 1 Motif 17 (ADAMTS17) gene. The CpG site, cg07394446, is a probe mapping to ADAMTS17.

At location 2360 is the SMAD Family Member 6 (SMAD6) gene. The CpG site, cg07124372, is a probe mapping to SMAD6.

At location 2388 is the Carbonic Anhydrase 12 (CA12) gene. The CpG site, cg10091775, is a probe mapping to CA12.

FIG. 23C illustrates a genomic diagram 2310 of chromosome 2. At location 2312 is the Bridging Integrator 1 (BIN1) gene. The BIN1 gene provides instructions for making a protein that is found in tissues throughout the body, where it interacts with a variety of other proteins. The BIN1 protein is involved in endocytosis as well as apoptosis, inflammation, and calcium homeostasis. The BIN1 protein may act as a tumor suppressor protein, preventing cells from growing and dividing too rapidly or in an uncontrolled way. In addition to its roles in tumor suppression and muscle development, multiple GWAS studies identified BIN1 as risk factor for late-onset Alzheimer's disease. While the exact pathogenic mechanism of BIN1 is still unknown, both high-risk variants and. DNA methylation have been suggested as mechanisms affecting BIN1 transcription and Alzheimer's Disease risk. The CpG site, cg27405400, is a probe mapping to the BIN1 gene.

At location 2328 is the Protein Tyrosine Phosphatase Receptor Type N (PTPRN) gene. This gene codes for a protein receptor involved in a multitude of processes including cell growth, differentiation, mitotic cycle, and oncogenic transformation. More specifically, this PTPRN plays a significant role in the signal transduction of multiple hormone pathways (neurotransmitters, insulin, and pituitary hormones). PTPRN expression levels are also used as a prognostic tool for hepatocellular carcinoma (negative outcome correlation). The CpG site, cg03545227, is a probe mapping in the protein tyrosine phosphatase receptor type N (PTPRN) gene.

FIG. 23D illustrates a genomic diagram 2314 of chromosome 19. At location 2316 is the Kruppel-Like Factor 2 (KLF2) gene. This gene encodes for a transcription factor (zinc finger protein) found in many different cell types. Expression starts early in mammalian development and plays a role in processes ranging from adipogenesis, embryonic erythropoiesis, epithelial integrity, inflammation, and t-cell viability. Its role in inflammation is of specific interest as chronic systemic inflammation highly correlates with aging and age associated disease. The downstream effect of KLF2 expression is the downregulation of inflammation and reduction of pro-inflammatory activity of nuclear factor kappa beta (NF-kB). In humans with chronic infections and inflammatory disease such as sepsis, rheumatoid arthritis, atherosclerosis a reduction of 30 to 50% in KLF2 levels has been observed. The CpG site, cg26842024, is a probe mapping to a CpG island in the Kruppel-Like Factor 2 (KLF2) gene.

At location 2412 is the Midnolin (MIDN) gene. The CpG site, cg07843568 is a probe mapping to MIDN.

FIG. 23E illustrates a genomic diagram 2330 of chromosome 1. At location 2332 is the Multiple EGF Like Domains 6 (MEGF6) gene. This gene plays a role in cell adhesion, motility and proliferation and is also involved in apoptotic cell phagocytosis. Mutations in this gene are associated with a predisposition to osteoporosis. The CpG site, cg23686029, is a probe mapping in the MEGF6 gene.

FIG. 23F illustrates a genomic diagram 2334 of chromosome 7. At location 2336 is the Kruppel-Like Factor 14 (KLF14) gene. This gene encodes a member of the Kruppel-like family of transcription factors and shows maternal monoallelic expression in a wide variety of tissues. The encoded protein functions as a transcriptional co-repressor and is induced by transforming growth factor-beta (TGF-beta) to repress TGF-beta receptor II gene expression. This gene exhibits imprinted expression from the maternal allele in embryonic and extra-embryonic tissues. Variations near this transcription factor are highly associated with coronary artery disease. The CpG site, cg08097417, is a probe mapping to the KLF14 gene.

At location 2368 is the Stromal Antigen 3 (STAG3/GPC2) gene. The CpG site, cg18691434, is a probe mapping to the STAG3/GPC2 gene. At location 2400 is the Huntingtin Interacting Protein 1 (HIP1) gene. The CpG site, cg13702357, is a probe mapping to the HIP1 gene. At location 2424 is the DPY19L2 Pseudogene 4 (DPY19L2P4) gene. The CpG site, cg22370005, is a probe mapping to the DPY19L2P4 gene.

FIG. 23G illustrates a genomic diagram 2338 of chromosome 6. At location 2340 is the Protein Phosphatase 1 Regulatory Subunit 18 (PPP1R18) gene. The protein that is encoded by this gene, phosphatase-1 (PP1), plays a role in glucose metabolism in the liver by controlling the activity of phosphorylase a that breaks down glycogen to release glucose in the blood stream. Furthermore, PP1 is also involved in diverse, essential cellular processes such as cell cycle progression, protein synthesis, muscle contraction, carbohydrate metabolism, transcription and neuronal signaling. In Alzheimer's disease, expression of PP1 is significantly reduced in both white and grey matter. The CpG site, cg23197007, is a probe that maps to the S-shore of the PPP1R18 gene.

At location 2352 is the TMEM181 gene. cg02447229 is a probe mapping to the TMEM181 gene. At location 2392 is the Zinc Finger and BTB Domain Containing 12 (ZBTB12) gene. The CpG site, cg06540876, is a probe mapping to the ZBTB12 gene.

FIG. 23H illustrates a genomic diagram 2342 of chromosome 17. At location 2344 is the dicarbonyl and L-xylulose reductase (DCXR) gene. One of its functions is to perform a chemical reaction that converts a sugar called L-xylulose to a molecule called xylitol. This reaction is one step in a process by which the body can use sugars for energy. There are two versions of L-xylulose reductase in the body, known as the major isoform and the minor isoform. The DCXR gene provides instructions for making the major isoform, which converts L-xylulose more efficiently than the minor isoform. It is unclear if the minor isoform is produced from the DCXR gene or another gene. Another function of the DCXR protein is to break down toxic compounds called alpha-dicarbonyl compounds. The DCXR protein is also one of several proteins that get attached to the surface of sperm cells as they mature. DCXR is involved in the interaction of a sperm cell with an egg cell during fertilization. The CpG site, cg07073120, is a probe that maps to the promotor region of the DCXR gene.

FIG. 23I illustrates a genomic diagram 2346 of chromosome 22. At location 2348 is the BCR Activator of RhoGEF and GTPase (BCR) gene. The CpG site, cg04028010, is a probe mapping to the BCR gene.

FIG. 23J illustrates a genomic diagram 2362 of chromosome 3. At location 2364 is the Myosin Light Chain Kinase (MYLK) gene. This gene codes for a myosin light chain kinase (MLCK), which is a calcium/calmodulin dependent enzyme active in smooth muscle tissue (involuntary muscle). It phosphorylates myosin light chains to facilitate their interaction with actin to produce muscle contraction. A second function of the MLCK protein is regulation of the epithelial tight junction. These are the gaps between the epithelial cells and their size is of major biological importance as this determines the selective permeability of the epithelial barrier. This selectivity allows absorption and secretion of nutrients and metabolites while protecting the sterile tissues against pathogens. A secondary promotor drives the expression of a second gene transcript: telokin. This small protein is identical to the c-terminus of the MYLK protein and helps stabilize unphosphorylated myosin filaments. Abnormal expression of MYLK have been observed in many inflammatory diseases such as, pancreatitis, asthma, inflammatory bowel disease.

At location 2372 is the Transglutaminase 4 (TGM4) gene. The CpG site, cg12112234, is a probe mapping to the TGM4 gene.

At location 2380 is the Scm Like With Four Mbt Domains 1 (SFMBT1) gene. The CpG site, cg03607117, is a probe mapping to the SFMBT1 gene.

At location 2404 is Nudix Hydrolase 16 (NUDT16P) gene. The CpG site, cg22575379, is a probe mapping to the NUDT16P gene.

FIG. 23K illustrates a genomic diagram 2382 of chromosome 12. At location 2384 is the Thyrotropin Releasing Hormone Degrading Enzyme (LOC283392/TRHDE) gene. The CpG site, cg13663218, is a probe mapping to the LOC283392/TRHDE gene.

FIG. 23L illustrates a genomic diagram 2396 of chromosome 8. At location 2396 is the Neurofilament Medium Chain (NEFM) gene. The CpG site, cg07502389, is a probe mapping to the NEFM gene.

FIG. 23M illustrates a genomic diagram 2418 of chromosome 5. At location 2420 is the Ring Finger Protein 180 (RNF180) gene. The RNF180 gene codes for RING-Type E3 Ubiquitin Transferase. RING-type E3s are implicated as tumor suppressors, oncogenes, and mediators of endocytosis, and play critical roles in complex multi-step processes such as DNA repair and activation of NF-κB a master regulator of inflammation. RING-type E3s and their substrates are implicated in a wide variety of human diseases ranging from viral infections to neurodegenerative disorders to cancer. The CpG site, cg23008153, is a probe mapping to the N-shore of the RNF180 gene.

FIG. 23N illustrates a genomic diagram 2374 of chromosome 18. At location 2376 is the SMAD Family Member 2 (SMAD2) gene. The CpG site, cg17243289, is a probe mapping to the SMAD2 gene.

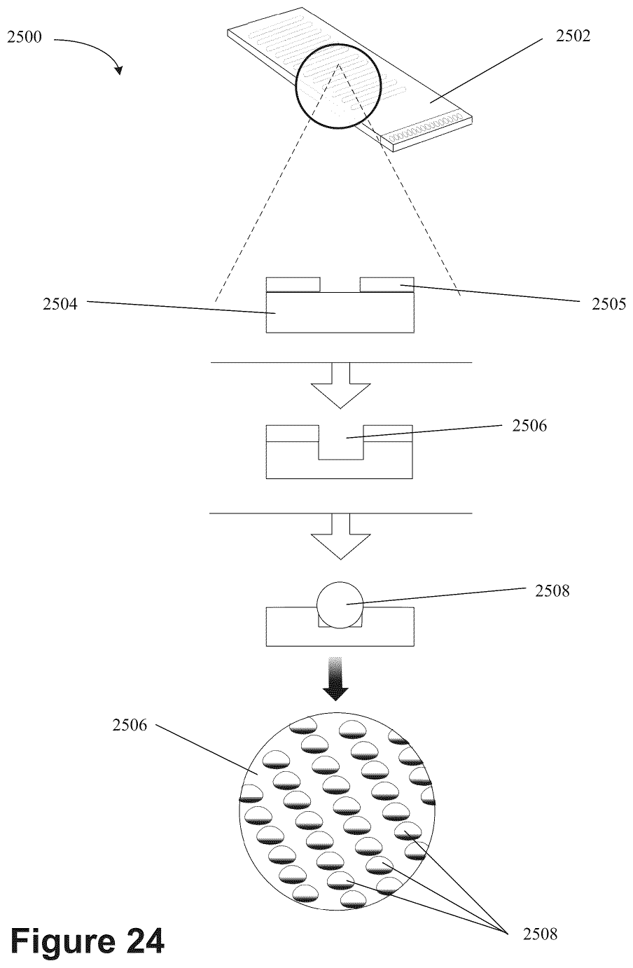

FIG. 24 is a diagram showing an example array chip 2502 used to detect methylation. Array chip 2502 includes a silicon wafer 2504 which is coated with a photo-resistant material 2505. Array chip 2502 includes a plurality of microwells 25025 disposed along its surface. Microwells 2506 are not covered by the photo-resistant material 2505 and extend depth-wise into the silicon wafer 2504. Each microwell 2506 houses one or more beads 2508. Beads 2508 are coated with multiple copies of an oligonucleotide probe targeting a specific location in the genome. As sample DNA fragments pass over beads 2508, each probe binds to a complementary sequence in the sample DNA, stopping proximate the location of interest. Thus, specific locations in the DNA sample can be targeted for analysis. For instance, locations corresponding to CpG sites can be targeted for methylation state analysis. Specific beads 2508 can be utilized to complete a methylation state analysis.

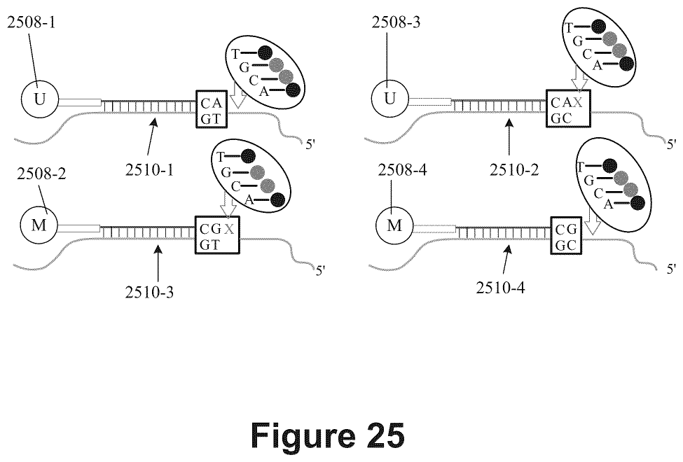

FIG. 25 is a diagram showing example beads 2508 that respond to methylated and unmethylated CpG sites. Before DNA is exposed to the methylation analysis beads 2508, the DNA is treated with sodium bisulfite. Sodium bisulfite converts cytosine into uracil, but leaves methylated cytosine (e.g., 5-methylcytosine) unaffected. The array chip 2502 interrogates these chemically differentiated locations using two site-specific probes, one bead type (U) (beads 2508-1, 2508-3) presents probes that are designed to match to an unmethylated site; the second bead type (M) (beads 2508-2, 2508-4) matches a methylated state. Single-base extension of the probes incorporates a labeled dideoxynucleotides (ddNTP), which is subsequently stained with a fluorescent reagent. The level of methylation for the interrogated location can be determined by calculating the ratio of the fluorescent signals from the methylated vs. unmethylated sites.

On the left side of this figure, the locus of interest is unmethylated. It matches perfectly with unmethylated bead probe 2508-1, enabling single-base extension and detection. The unmethylated locus has a single-base mismatch to the methylated bead probe 2508-2, inhibiting extension that results in a low signal on the array. If the CpG locus of interest is methylated, the reverse occurs: the methylated bead 2508-4 type will display a signal, and the unmethylated bead 2508-3 type will show a low signal on the array. If the locus has an intermediate methylation state, both probes will match the target site and will be extended. Methylation status of the CpG site is determined by a p-value calculation, which is the ratio of the fluorescent signals from the methylated beads to the total locus intensity. The array chip containing the beads 2508 can be read by an array scanning device, such as the iScan™ System provided by Illumina or the NexSeq™ 550 System provided by Illumina.

FIGS. 24-25 disclose one system to detect methylation. The present disclosure also explicitly contemplates using other methods to detect the methylation status of CpG sites.

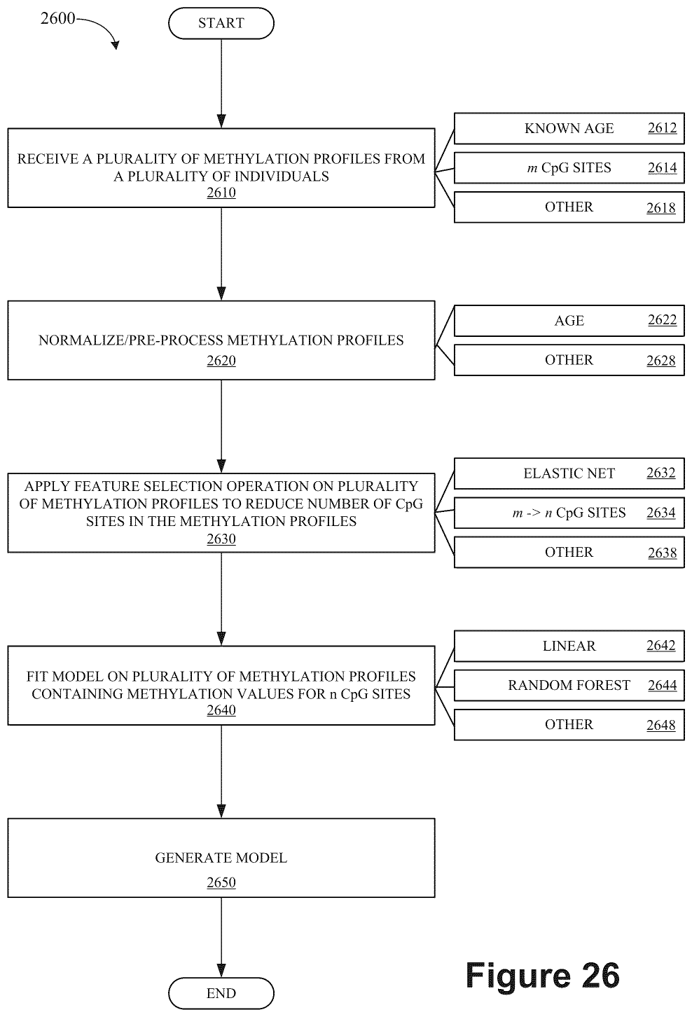

FIG. 26 illustrates a flow diagram showing an example operation 2600 of training an epigenetic age predicting model. Operation 2600 begins at block 2610 where a plurality of methylation profiles from a plurality of individuals are received. A methylation profile contains methylation values for a number of CpG sites of the individual. Methylation values can be in a number of different formats. An example format is a decimal between zero and one (0-value), where zero is fully unmethylated and one is fully methylated. These methylation profiles for the individuals can be derived from an array described above with respect to FIGS. 5-6.

As indicated by block 2612, the plurality of individuals' ages are known. This known age can be used to generate the model. For example, the plurality of methylation values in each profile can be used as an input vector and the known age is the scalar output value.

As indicated by block 2614, the number of CpG sites in each of the plurality of methylation profiles is m. The quantity m can be a variety of different numbers. For example, m can correspond to a resolution of the methylation analysis method used on the plurality of individuals. For instance, Illumina provides methylation microarrays that detect ˜850,000 CpG sites (Infinium methylation EPIC array) or, for instance, Illumina provides methylation sequencing for 3.3 million or 36 million CpG sites.

Operation 2600 proceeds at block 2620 where the plurality of methylation profiles received in block 2610 are normalized or otherwise pre-processed. As indicated by block 2622 the methylation profiles can be normalized based on age. For instance, a specific age range may be overrepresented in the plurality of profiles, therefore the profiles from this age range may need to be weighted less or their chance of being sampled made less likely. In the alternate, a specific age range may be underrepresented in the plurality of profiles, therefore the profiles from this age range may need to be weighted higher or their chance of being sampled made more likely.

As indicated by block 2624, the plurality of methylation profiles may be curated based on a quality metric. For example, methylation profiles that have a quality metric lower than a threshold are discarded or weighted less than methylation profiles of a higher quality metric. In some examples, the quality metric is indicative of the accuracy of the methylation process. In some examples, the quality metric is indicative of the quality of the sample used to generate the particular methylation profile. In some examples, the quality metric is indicative of the quality of the test used to generate the particular methylation profile. In other examples, the quality metric may be indicative of other factors relating the particular methylation profile. As indicated by block 2628, the plurality of methylation profiles can be normalized or pre-processed in other ways as well.

Operation 2600 proceeds at block 2630 where a feature selection operation is applied on the plurality of methylation profiles. In some examples, the feature selection is applied on the plurality of methylation profiles after they are pre-processed. In some examples, the feature selection operation is applied on the plurality of methylation profiles received in block 2610.

As indicated by block 2632, the feature selection operation applied on the plurality of methylation includes elastic net regression. Elastic net regression combines L1 penalties from lasso regression and L2 penalties from ridge regression to reduce the number of CpG sites.

As indicated by block 2634, the feature selection applied reduces the number of applicable CpG sites from m to n sites. This reduction can balance dimensionality when there are small number of methylation profiles, but each profile has a large amount of CpG site methylation values. In some examples, m is greater than 100,000. In some examples, m is greater than 400,000. In some examples, m is greater than 800,000. In some examples, n is less than 200. In some examples, n is less than 100. In some examples, n is less than 50.

Operation 2600 proceeds at block 2640 where a model is fit on the plurality of methylation profiles. In some implementations, the model is fit on the plurality of methylation profiles, but only considers the CpG sites in the subset of n CpG sites. In some implementations, the model is fit on all m CpG sites. As indicated by block 2642, the model can include a linear regression model. As indicated by block 2644, the model can include a random forest model. As indicated by block 2648, the model can include a different type of model as well.

Operation 2600 proceeds at block 2650 where the model is generated. In some examples, the model is generated as one or more files that can be imported by other systems which can further train the model or use the model for predictions.

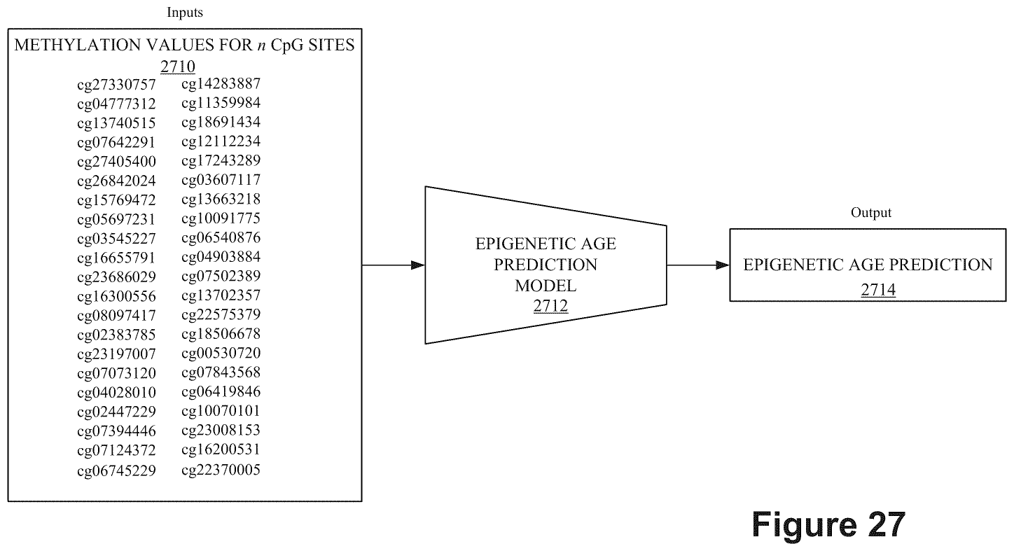

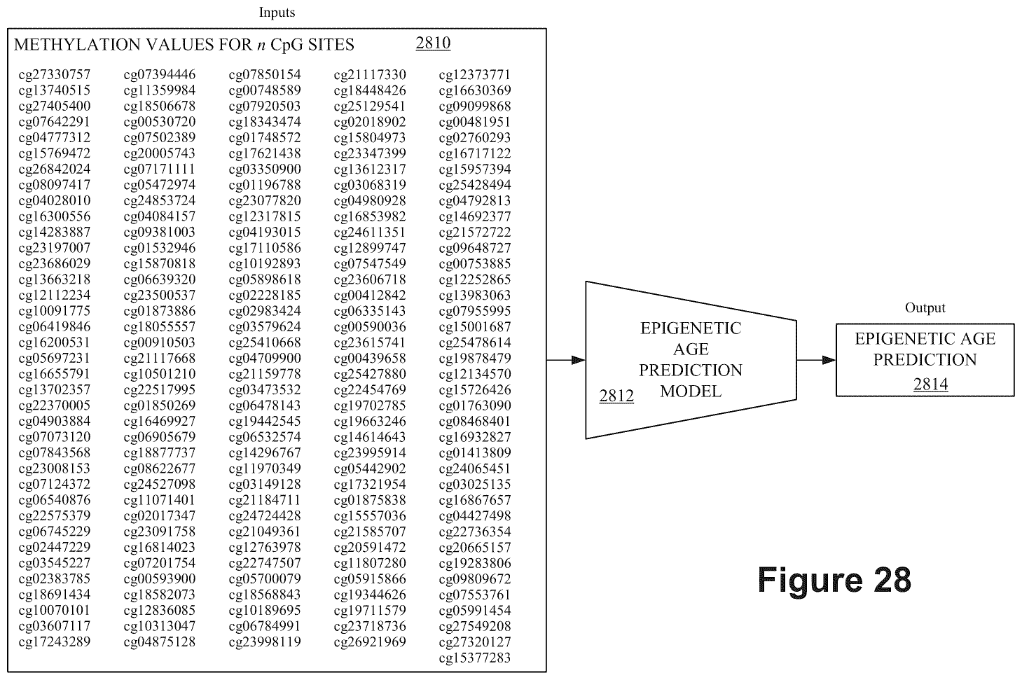

FIGS. 27-28 are flow diagrams showing example operations of predicting an epigenetic age. Operation 2700 begins at block 2710 where the input values are received. As shown, there are 42 input values corresponding to the methylation values at 42 different CpG cites. These CpG sites are identified by their CpG cluster identifier number. In some implementations, only a subset of the shown CpG sites is used as inputs. In some implementations, the shown CpG sites are in order of their feature importance to the model (e.g., in the case of a random forest model) or in order of the absolute value of their coefficient (e.g., in a linear model). In one implementation, the input values are derived from a methylation analysis on a sample of human blood.

At block 2712, the inputs are input into the epigenetic age prediction model. As noted above this model could include a linear regression model (e.g., where each input forms part of a linear expression), random forest model (e.g., where each input forms part of one or more decision trees), or some other type of model. At block 2714, the model outputs an epigenetic age prediction. In some implementations, the model also outputs a confidence score.

Operation 2800 begins at block 2810 where the input values are received. As shown, there are 1276 input values corresponding to the methylation values at 1276 different CpG cites. These CpG sites are identified by their CpG cluster identifier number. In some implementations, only a subset of the shown CpG sites is used as inputs. In some implementations, the shown CpG sites are in order of their feature importance to the model (e.g., in the case of a random forest model) or in order of the absolute value of their coefficient (e.g., in a linear model). In one implementation, the input values are derived from a methylation analysis on a sample of human blood.

At block 2812, the inputs are input into the epigenetic age prediction model. As noted above this model could include a linear regression model (e.g., where each input forms part of a linear expression), random forest model (e.g., where each input forms part of one or more decision trees), or some other type of model. At block 2814, the model outputs an epigenetic age prediction. In some implementations, the model also outputs a confidence score.

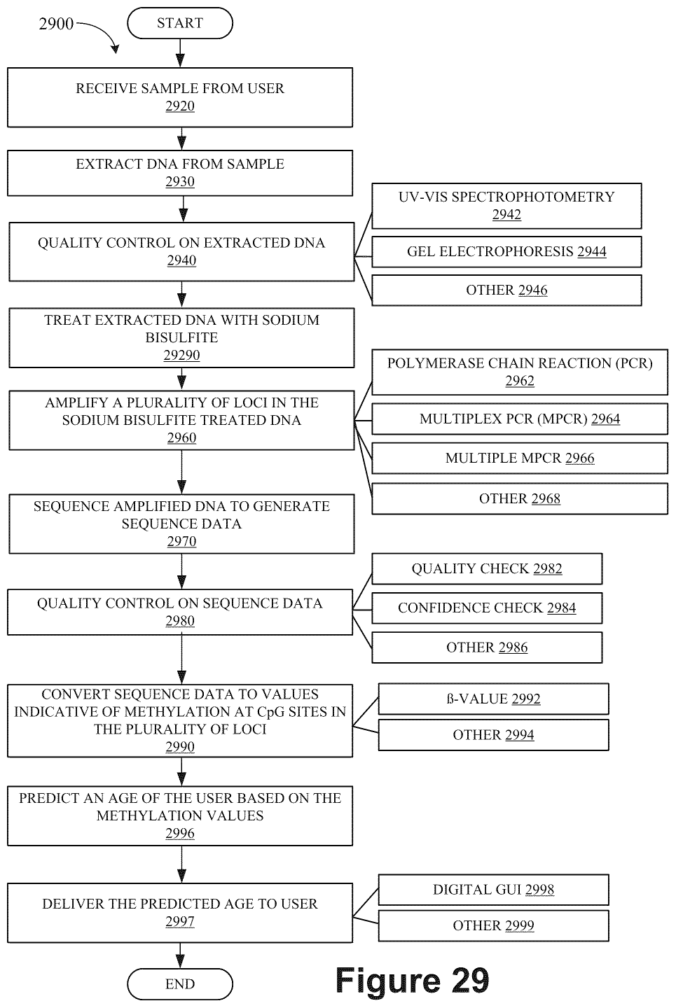

FIG. 29 illustrates a flow diagram showing an example operation 2900 of processing a sample to provide the epigenetic age to a user. Operation 2900 begins at block 2920 where a sample from the user is received. The sample may be received by a delivery or shipment in which the user generates a sample and prepares it for transit. For example, the sample may be received in a similar manner to the method discussed below with respect to FIG. 7. In another example, the user may directly deliver the generated sample to the site of DNA extraction, or to a facility that prepares it for preservation during long-term transit. In one embodiment, the sample may be a blood sample. However, it is expressly contemplated that other samples may be received and processed as well.

The operation proceeds at block 2930, where DNA is extracted from the sample. The process of DNA extraction includes breaking, or lysing, the cells contained in the sample to release the DNA. Cells may be lysed, for example, by using a lysis buffer or other solution suitable for breaking down cellular membranes while retaining DNA quality. After lysing the cells, the DNA is solubilized and separated from cellular debris. Solubilizing may include, for example, resuspending the lysed DNA in a buffer and/or soluble solution. The cellular debris removed may include proteins, cellular membranes, lipids, or other cellular components that may hinder DNA purity. Separation of the DNA from cellular debris may be done by a variety of methods, including filtration, protein degradation, or any combination thereof. In one embodiment, DNA may be extracted manually. However, in other embodiments, a commercially available DNA extraction kit may be utilized as well.

Operation 2900 proceeds at block 2940, where quality control is performed on the extracted DNA to receive quality-controlled DNA (QCDNA). Quality control may, for example, include obtaining absorbance measurements of the extracted DNA by ultraviolet-visible (UV-Vis) spectrophotometry 2942. The absorbance measurements may be indicative of extracted DNA quality. Additionally, the measurements provided by UV-Vis spectrophotometry 2942 may be indicative of the concentration of DNA acquired in the extraction process of the sample. Alternatively, or additionally, quality control may include performing gel electrophoresis on the extracted DNA 2944 to provide an indication of DNA quality. For example, DNA may be observed as it travels down the gel to indicate that the DNA is of an expected size and/or quality. Additionally, it is expressly contemplated that other forms of quality control may implemented as well, as indicated by block 2946. In one example, quality control may include combining subsequent extracted DNA aliquots in order to achieve a desired concentration level. Additionally, quality control may include combining the extracted DNA of subsequent samples acquired by the user. In another embodiment, quality control may include diluting the extracted DNA in order to achieve a desired DNA concentration.

Operation 2900 proceeds at block 2950 with treating the extracted DNA with sodium bisulfite. Sodium bisulfite converts cytosine into uracil in the extracted DNA sample, but leaves methylated cytosine (e.g., 29-methylcytosine) unaffected. This, in turn, allows for methylated cytosine to be differentiated from unmethylated cytosine in DNA analysis. In this way, the remaining methylated cytosine within the DNA segment of interest will remain detectable and observable, while unmethylated cytosine will not be in a chemical configuration to distort DNA analysis. Additionally, while sodium bisulfite is used in the present example, it is expressly contemplated that other means of treating the extracted DNA may be utilized as well (e.g., enzyme-based conversion or other methods).

Operation 2900 proceeds at block 2960, where a plurality of loci in the sodium bisulfite treated DNA is amplified. A locus is defined as a specific position on the DNA where a particular gene or DNA sequence is located. For example, the plurality of loci may be positions including one or more CpG sites of interest. DNA amplification involves generating multiple copies of the loci in one or more DNA segments of interest. As indicated by block 2962, amplification of the plurality of loci in the sodium bisulfate treated DNA may occur via polymerase chain reaction (PCR). PCR is a technique involving a set of primers, which are short segments of DNA that define the specific DNA sequence of interest and prepare it for amplification. Once the specific primers indicative of the site(s) to be amplified have been added, a replication enzyme, DNA polymerase, attaches to the site of amplification at the primers, and amplification of the loci in the treated DNA begins under standard reaction conditions. In this way, the one or more sets of primers serve as a marker for the start and end of a target sequence to be amplified.

As indicated by block 2964 and further described below with respect to FIG. 6, amplification of the plurality of loci in the sodium bisulfite treated DNA may also occur by multiplex PCR (MPCR). Whereas standard PCR typically involves the use of one pair of primers, MPCR involves the use of two or more primer pairs in the reaction. In this way, a plurality of loci in the DNA segment of interest may be amplified in a single reaction mixture. The plurality of loci may include, for example, a plurality of CpG sites. The plurality of CpG sites within the DNA segment may be amplified in the same reaction mixture using a plurality of primers corresponding to the CpG sites of interest. Additionally, as indicated by block 2966, multiple MPCR reactions may be utilized in a single reaction mixture, wherein multiple sites of interest may be amplified in multiple segments of DNA within the mixture. For example, different segments of DNA including a plurality of different CpG sites of interest may be amplified via MPCR in a single reaction mixture to generate amplified DNA corresponding to the plurality of CpG sites. In another example, a plurality segments may be amplified via MPCR in multiple subsequent reactions such that the sites of interest are amplified while the possibility of cross hybridization and/or mis-priming is minimized. In this way, multiple DNA segments of interest, such as those including CpG sites, may be amplified in one or few reactions, thus increasing efficiency and preventing the need to undergo a new reaction per site. Additionally, it is expressly contemplated that other modes of amplifying a plurality of loci can be utilized as well, as indicated by block 2968.

The operation proceeds at block 2970, where the amplified DNA is sequenced to generate sequence data. Sequencing is the process of determining the specific order, or sequence, of nucleotide bases within the one or more segments of amplified DNA. Sequencing may occur, for example, using a DNA sequencing system, in which the amplified DNA is processed to emit one or more signals indicative of the chain of nucleotides present within the sample. As indicated above with respect to block 2950, the extracted DNA is treated with sodium bisulfite prior to amplification. In this way, the signals emitted by the sequencing system indicative of cytosine correlate to methylated cytosine (e.g., 29-methylcytosine) rather than unmethylated. Thus, in an example where one or more CpG sites is amplified and consecutively sequenced, the sequence data received may be indicative of the degree of methylation relative to said CpG sites.

The operation proceeds at block 2980, where quality control is performed on the received sequence data. In an example where MPCR is utilized involving a plurality of primers, quality control enables confirmation that no errors have occurred during amplification, such as cross hybridization and/or mis-priming. Quality control may include, for example, a quality check, as indicated by block 2982. The quality check may include ensuring that the one or more segments of amplified DNA are of proper length. Additionally, the quality check may include comparing the one or more segments of amplified DNA with a reference to ensure that the proper DNA segment was amplified, and no random sequencing occurred beyond the desired segment(s) of interest. In one embodiment, quality control may further include a confidence check, as indicated by block 2984. The confidence check may include, for example, providing a confidence metric indicative of the error rate between the received sequencing data relative to the one or more amplified sites and a reference. Additionally, it is expressly contemplated that other forms of quality control can occur as well, as indicated by block 2986.

The operation proceeds at block 2990, where the sequence data is converted to values indicative of methylation at the CpG sites in the plurality of loci. Sequence data may be converted to methylation values in a number of different formats. For example, one possible format is the conversion of sequence data indicative of methylation at CpG sites of interest to a decimal between zero and one (β-value), as indicated at block 2992, where zero is fully unmethylated and one is fully methylated. Additionally, it is expressly contemplated that the sequence data can be converted to other formats as well, as indicated by block 2994.

The operation proceeds at block 3000, where an epigenetic age of the user is predicted based on the methylation values. The epigenetic age may be predicted by, for example, inputting the methylation values based on the sequence data into an epigenetic age prediction model, such as model 2712 discussed below with respect to FIG. 27. In some examples, the methylation values that are input into the model to predict the epigenetic age of the user are based on sequencing data corresponding to the 42 sites indicated in FIG. 27. Additionally, in some implementations, the methylation values input, corresponding to the amplified CpG sites, are in order of their feature importance to the model or in order of the absolute value of their coefficient. Further, in other examples, less methylation values corresponding to less than 42 amplified CpG sites may be input as well.

The operation proceeds at block 3010, where the predicated epigenetic age is delivered to the user. The predicated age can be delivered, for example, using a digital graphical user interface (GUI) 3012. Additionally, it is expressly contemplated that the predicted age may be delivered to a user by other means as well, as indicated by block 3014.

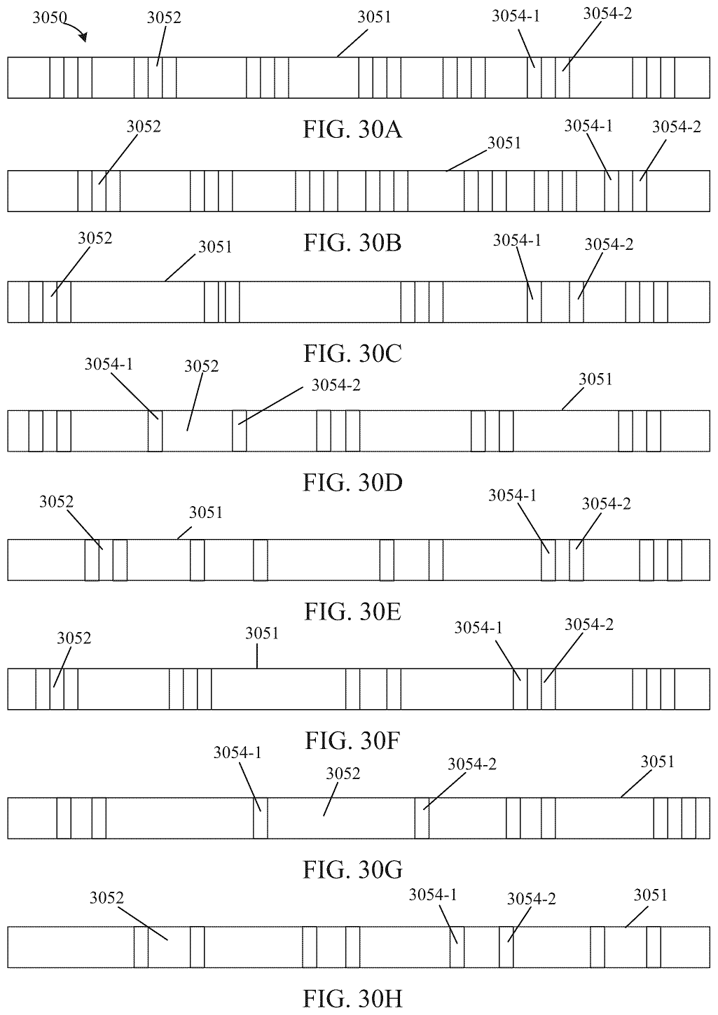

FIGS. 30A-30H illustrate diagrams showing example amplifications of one or more portions of DNA. FIGS. 30A-30H recite similar features and like components are numbered accordingly. DNA portion 3050 illustratively includes DNA strand 3051. As recited above, DNA strand 3051 comprises a plurality of nucleotide base pairs (not shown), which extend along the length of strand 3051. For example, one or more sets of a cytosine nucleotide followed by a guanine nucleotide (e.g. a CpG site) may be disposed within strand 3051. In one embodiment, strand 3051 may be a DNA segment of interest and have a length of nucleotides relative to the particular DNA segment. However, in other embodiments, strand 3051 may be the length of the human genome.

DNA portion 3050 further includes a plurality of primer pairs 3054-1 and 3054-2 generally disposed over varying lengths of strand 3051. As illustrated in FIGS. 30A-30H, each primer 3054-1 has a consecutive primer 3054-2, which acts as the start and stop marker for amplification. In this way, primers 3054-1 and 3054-2 act as a primer pair. As illustrated, the one or more primer pairs 3054-1 and 3054-2 define amplification site 3052. Amplification site 3052 includes a section of strand 3051 of interest to be amplified. For example, site 3052 may include one or more CpG sites of interest to be amplified. Amplification site 3052 can be of varying lengths, as defined by the placement of primer pairs 3054-1 and 3054-2. For example, the amplification site 3052 shown in FIG. 30G is of a larger length than site 3052 shown in FIG. 30H relative to the placement of primer pairs 3054-1 (3054-2).

As noted in FIGS. 30A-30H, each strand 3051 can include a plurality of primer pairs 3054-1 and 3054-2, and thus a plurality of amplification sites 3052. For instance, FIG. 30A includes 7 primer pairs 3054-1 and 3054-2, and therefore 7 amplification sites 3052. FIG. 30B includes 7 primer pairs 3054-1 and 3054-2, and therefore 7 amplification sites 3052. FIG. 30C includes 5 primer pairs 3054-1 and 3054-2, and therefore 5 amplification sites 3052. FIG. 30D includes 5 primer pairs 3054-1 and 3054-2, and therefore 5 amplification sites 3052. FIG. 30E includes 5 primer pairs 3054-1 and 3054-2, and therefore 5 amplification sites 3052. FIG. 30F includes 5 primer pairs 3054-1 and 3054-2, and therefore 5 amplification sites 3052. FIG. 30G includes 4 primer pairs 3054-1 and 3054-2, and therefore 4 amplification sites 3052. FIG. 30H includes 4 primer pairs 3054-1 and 3054-2, and therefore 4 amplification sites 3052. Additionally, it is expressly contemplated that any of FIGS. 30A-30H could include a different number of primer pairs and therefore a different number of amplification sites as well.

In one example, FIGS. 30A-30H may be representative of multiple MPCR reactions to amplify a plurality of sites corresponding to a plurality of CpG sites. For example, as shown, 8 separate MPCRs may be completed to amplify a total of 42 sites. The 42 sites may correspond to, for example, the 42 CpG sites set forth below with respect to FIG. 27. Additionally, it is expressly contemplated that less MPCR reactions may be completed to amplify the same 42 CpG sites, or a different number of CpG sites. For instance, a different number of primer pairs 3054-1 (3054-2) may be added to a single MPCR to increase the amplification sites while reducing the subsequent MPCRs needed. In another example, less than the 42 listed CpG sites may be desired for epigenetic age determination, in which case a lower number of primer pairs 3054-1 (3054-2) may be added relative to the number of CpG sites to be amplified.

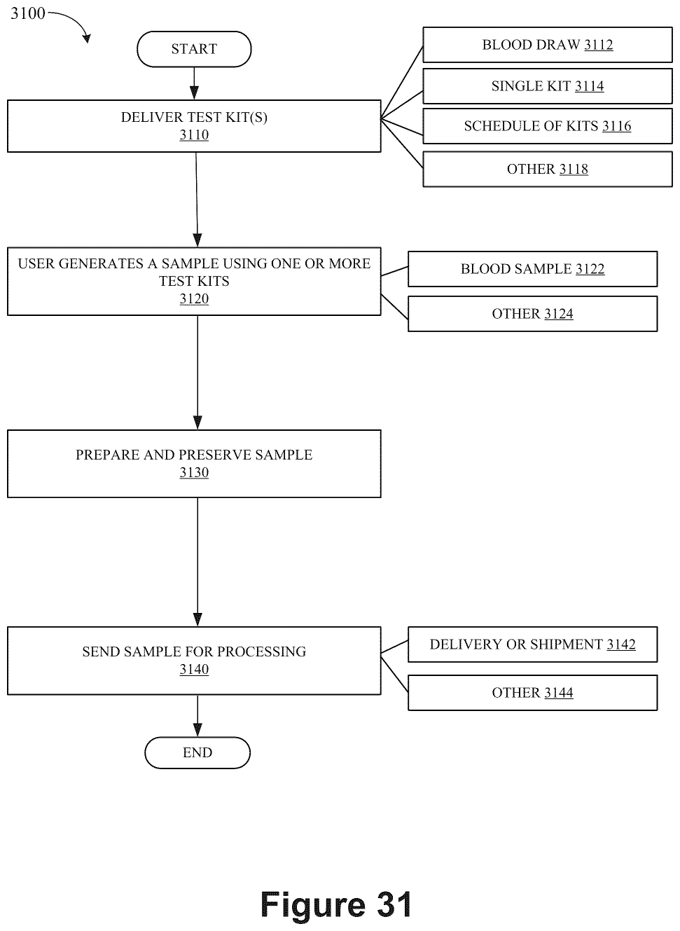

FIG. 31 illustrates a flow diagram showing an example operation of acquiring a sample from a user. Operation 3100 begins at block 3110 where one or more test kits are delivered to the user. A test kit provides the necessary tools and/or protocols for a user to obtain a sample to be processed. The sample may be obtained in a number of different formats. For example, the sample may be a blood sample obtained by blood draw 3112. Blood draw 3112 may include any device suitable for safely obtaining blood and retaining sample purity. For example, blood draw 3112 may include a non-invasive blood extraction device included in the test kit, in which a user operates the device to obtain the sample. In another example, blood draw 3112 may include a vial and syringe. Alternatively, blood draw 3112 may include a fingerstick. Additionally, it is expressly contemplated that other samples may be obtained by the user as well. For instance, the sample may be a saliva sample.

As indicated by block 3114, delivering the test kit may include delivering a single kit. However, in other examples, the test kit may include multiple test kits. For instance, multiple test kits may be included such that the user may obtain multiple samples to ensure a sample of sufficient quality is acquired. Additionally, as indicated by block 3116, delivering the test kit may include delivering a schedule of kits. For example, a plurality of test kits may be scheduled for delivery in specific intervals over time such that a range of samples may be processed to observe the predicted epigenetic age of the user over time. In another example, the schedule of test kits may be delivered to a user at one time with instructions on when to generate and prepare the next consecutive sample. Additionally, the test kit may be some other form of test kit, as indicated by block 3118.

Operation 3100 proceeds at block 3120 where a user generates a sample using the one or more test kits. As indicated by block 3122 and further discussed above, the sample generated by the user may be a blood sample. For instance, the sample may be generated using a blood extraction device. The blood sample may be disposed in any vessel suitable for collection. For example, the vessel may be a collection tube provided in the one or more test kits. In one embodiment, the vessel may include one or more indicators to indicate to the user when a sufficient volume of sample has been acquired. For example, the indicator may be a fill line on the lower portion of a collection tube that may indicate to a user that enough sample is collected. In another example, the fill line may be at the middle or near the top of the collection tube. Additionally, the collection tube may contain multiple fill lines corresponding to different volumes of the sample to be collected. For example, a first line on collection tube may indicate that enough sample has been collected for DNA extraction, and an additional line above the first line may indicate that an additional sample aliquot has been collected for a consecutive DNA extraction. Additionally, while a blood sample is described as being generated above, it is expressly contemplated that other samples may be generated by the user as well, as indicated by block 3124.

Operation 3100 proceeds at block 3130 where the sample is preserved and prepared for transit. Preservation of the sample may include sealing the sample in a vessel suitable for transportation. For example, a sample collected in a collection tube may be sealed with a collection tube cap. In another embodiment, preparation of the sample may include freezing the sample prior to transit. Additionally, it is expressly contemplated that other forms of sample preparation can occur as well.

Operation 3100 proceeds at block 3140 where the sample is sent for processing. The sample may be sent by a delivery or shipment, as indicated by block 3142, in which the user generates a sample and stores it and any other necessary contents in an appropriate container for transit. In one example, the container for shipment may be provided in the one or more test kits delivered to the user. However, it is expressly contemplated that other forms of sample transportation can occur as well, as indicated by block 3144. For example, the user may directly deliver the generated sample to the site where DNA extraction occurs. Alternatively, the user may deliver the sample to a facility that prepares it for preservation during long-term transit.

### Computer System

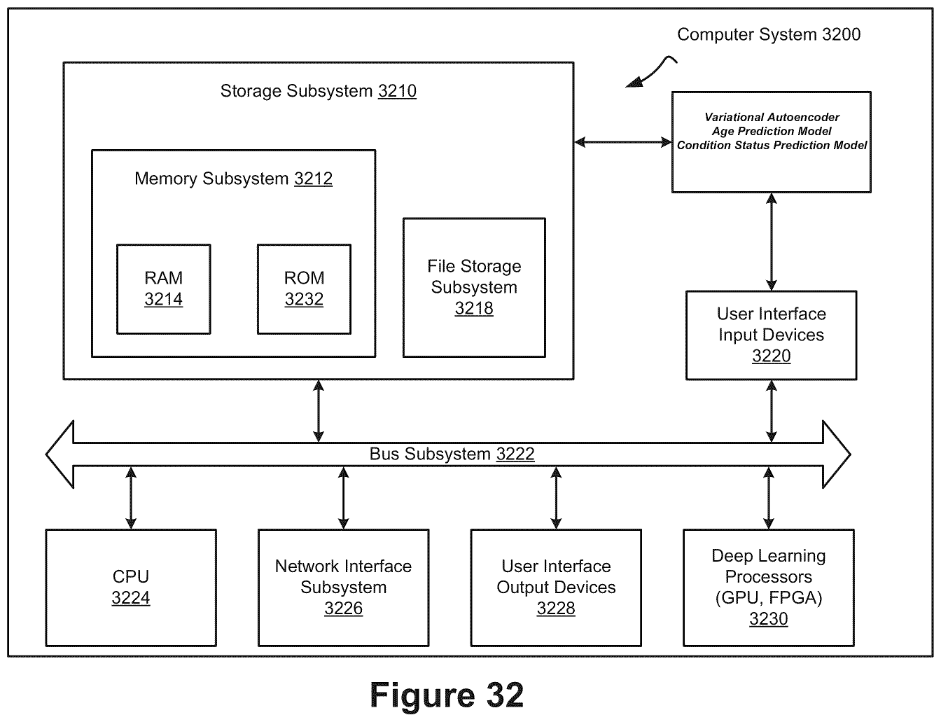

FIG. 32 shows an example computer system 3200 that can be used to implement the technology disclosed. Computer system 3200 includes at least one central processing unit (CPU) 3272 that communicates with a number of peripheral devices via bus subsystem 3255. These peripheral devices can include a storage subsystem 3210 including, for example, memory devices and a file storage subsystem 3236, user interface input devices 3238, user interface output devices 3276, and a network interface subsystem 3274. The input and output devices allow user interaction with computer system 3200. Network interface subsystem 3274 provides an interface to outside networks, including an interface to corresponding interface devices in other computer systems.

In one implementation, the VAE is communicably linked to the storage subsystem 3210 and the user interface input devices 3238.

User interface input devices 3238 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 3200.

User interface output devices 3276 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 3200 to the user or to another machine or computer system.

Storage subsystem 3210 stores programming and data constructs that provide the functionality of some or all of the modules and methods described herein. These software modules are generally executed by processors 3278.

Processors 3278 can be graphics processing units (GPUs), field-programmable gate arrays (FPGAs), application-specific integrated circuits (ASICs), and/or coarse-grained reconfigurable architectures (CGRAs). Processors 3278 can be hosted by a deep learning cloud platform such as Google Cloud Platform™, Xilinx™, and Cirrascale™. Examples of processors 3278 include Google's Tensor Processing Unit (TPU)™, rackmount solutions like GX4 Rackmount Series™, GX32 Rackmount Series™, NVIDIA DGX-1™, Microsoft' Stratix V FPGA™, Graphcore's Intelligent Processor Unit (IPU)™, Qualcomm's Zeroth Platform™ with Snapdragon Processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 3222 used in the storage subsystem 3210 can include a number of memories including a main random access memory (RAM) 3232 for storage of instructions and data during program execution and a read only memory (ROM) 3234 in which fixed instructions are stored. A file storage subsystem 3236 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 3236 in the storage subsystem 3210, or in other machines accessible by the processor.

Bus subsystem 3255 provides a mechanism for letting the various components and subsystems of computer system 3200 communicate with each other as intended. Although bus subsystem 3255 is shown schematically as a single bus, alternative implementations of the bus subsystem can use multiple busses.

Computer system 3200 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 3200 depicted in FIG. 32 is intended only as a specific example for purposes of illustrating the preferred implementations of the present invention. Many other configurations of computer system 3200 are possible having more or less components than the computer system depicted in FIG. 32.

### Clauses

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed or elements thereof can be implemented in the form of a computer product, including a non-transitory computer readable storage medium with computer usable program code for performing the method steps indicated. Furthermore, one or more implementations and clauses of the technology disclosed or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:

### Clause Set 1—“Non-Sequence Altering Change Latent Space”

1. A system, comprising:
a variational autoencoder comprising an encoder neural network and a decoder neural network,
   the encoder neural network configured to process a plurality of non-sequence altering change marker inputs, and to encode the plurality of non-sequence altering change marker inputs in a non-sequence altering change latent space,
      wherein the non-sequence altering change latent space comprises respective feature embeddings that compress respective non-sequence altering change marker inputs in the plurality of non-sequence altering change marker inputs into a jointly Gaussian distribution; and
   the decoder neural network configured to sample the respective feature embeddings from the non-sequence altering change latent space, and to decode the respective feature embeddings into respective outputs that are respective reconstructions of the respective non-sequence altering change marker inputs.

2. The system of clause 1, wherein each non-sequence altering change marker input in the plurality of non-sequence altering change marker inputs specifies a plurality of modification sites in a corresponding sample.

3. The system of clause 2, wherein each non-sequence altering change marker input identifies respective modification sites in the plurality of modification sites using respective modification values.

4. The system of clause 3, wherein the encoder neural network is configured to generate the non-sequence altering change latent space by combining the respective modification values in a weighted, nonlinear pattern, and thereby selecting signal features from the respective modification values, and not selecting noise features from the respective modification values.

5. The system of clause 1, wherein the decoder neural network is configured to randomly sample the feature embeddings from the non-sequence altering change latent space.

6. The system of clause 1, wherein the encoder neural network is a multi-layer neural network that is interspersed with batch normalization functions and nonlinear activation functions.

7. The system of clause 1, wherein the decoder neural network is a multi-layer neural network that is interspersed with batch normalization functions and nonlinear activation functions.

8. The system of clause 1, further configured to generate the non-sequence altering change latent space by repeatedly training the variational autoencoder on the plurality of non-sequence altering change marker inputs over multiple epochs.

9. The system of clause 8, wherein the variational autoencoder is trained using an ADAM optimizer with a Mean Squared Error (MSE) or Binary Cross Entropy (BCE) loss function and a Kullback-Leibler divergence (KLD) loss function.

10. The system of clause 9, wherein the MSE or BSE loss function measures a reconstruction loss based on comparing the respective outputs to the respective non-sequence altering change marker inputs.

11. The system of clause 9, wherein the KLD loss function measures a distance between a distribution of the non-sequence altering change latent space and a second distribution.

12. The system of clause 11, wherein the second distribution is a Gaussian distribution, thereby causing the KLD loss function to require the non-sequence altering change latent space to approximate a normal random variable.

13. The system of clause 11, further configured to use a Monte Carlo approximation of the KLD loss function.

14. The system of clause 13, wherein the Monte Carlo approximation is distribution agnostic and calculates the distance without making any prior assumptions about any underlying distribution.

15. The system of clause 9, wherein the variational autoencoder is trained using a loss function that is defined as the MSE or BSE loss function+w*the KLD loss function, where w is a KLD weight.

16. The system of clause 15, wherein the KLD weight is set to a specific value between zero and unity.

17. The system of clause 15, wherein the KLD weight varies between zero and unity, following a cyclical sigmoid annealing scheduling to mitigate KLD vanishing.

18. The system of clause 1, wherein the feature embeddings are biology-related feature embeddings.

19. The system of clause 18, wherein the feature embeddings are age-related feature embeddings.

20. The system of clause 1, wherein a non-sequence altering change age prediction model is configured to process the feature embeddings in the non-sequence altering change latent space through a plurality of fully connected layers and generate non-sequence altering change age predictions.

21. The system of clause 20, wherein the non-sequence altering change age prediction model is interspersed with dropout functions and nonlinear activation functions.

22. The system of clause 20, wherein the non-sequence altering change age prediction model is further configured to:
select, from the non-sequence altering change latent space, a particular feature embedding of a particular non-sequence altering change marker input;
process the particular feature embedding through the plurality of fully connected layers; and
generate a non-sequence altering change age prediction for a particular individual that sourced the particular non-sequence altering change marker input.

23. The system of clause 20, wherein the non-sequence altering change age prediction model is trained on the respective feature embeddings labelled with respective known non-sequence altering change ages of respective individuals that sourced the respective non-sequence altering change marker inputs.

24. The system of clause 23, wherein the non-sequence altering change age prediction model is trained using an ADAM optimizer along with a weighted MSE or BSE loss function, where an error on each sample is weighted according to its age class weight.

25. The system of clause 1, wherein a condition status prediction model is configured to process the feature embeddings in the non-sequence altering change latent space and generate condition status predictions.

26. The system of clause 25, wherein the condition status prediction model is a binary of multi-class classification model.

27. The system of clause 25, wherein the condition status prediction model is further configured to:
select, from the non-sequence altering change latent space, a particular feature embedding of a particular non-sequence altering change marker input;
process the particular feature embedding; and
generate a condition status prediction for a particular individual that sourced the particular non-sequence altering change marker input.

28. The system of clause 25, wherein the condition status prediction model is trained on the respective feature embeddings labelled with respective known condition statuses of respective individuals that sourced the respective non-sequence altering change marker inputs.

29. The system of clause 28, wherein the condition status prediction model is trained using an ADAM optimizer along with a weighted MSE or BSE loss function, where an error on each sample is weighted according to its age class weight.

30. A system, comprising:
a variational autoencoder comprising an encoder neural network and a decoder neural network,
   the encoder neural network configured to process a plurality of non-sequence altering change marker inputs, and to encode the plurality of non-sequence altering change marker inputs in a non-sequence altering change latent space,
      wherein the non-sequence altering change latent space comprises respective feature embeddings that compress respective non-sequence altering change marker inputs in the plurality of non-sequence altering change marker inputs into a jointly Gaussian distribution; and
   the decoder neural network configured to sample the respective feature embeddings from the non-sequence altering change latent space, and to decode the respective feature embeddings into respective outputs that are respective reconstructions of the respective non-sequence altering change marker inputs; and
a non-sequence altering change age prediction model configured to process the respective feature embeddings through a plurality of fully connected layers and generate respective non-sequence altering change age predictions for respective individuals that sourced the respective non-sequence altering change marker inputs.

31. A system, comprising:
a variational autoencoder comprising an encoder neural network and a decoder neural network,
   the encoder neural network configured to process a plurality of non-sequence altering change marker inputs, and to encode the plurality of non-sequence altering change marker inputs in a non-sequence altering change latent space,
      wherein the non-sequence altering change latent space comprises respective feature embeddings that compress respective non-sequence altering change marker inputs in the plurality of non-sequence altering change marker inputs into a jointly Gaussian distribution; and
   the decoder neural network configured to sample the respective feature embeddings from the non-sequence altering change latent space, and to decode the respective feature embeddings into respective outputs that are respective reconstructions of the respective non-sequence altering change marker inputs; and
a condition status prediction model configured to process the respective feature embeddings and generate respective condition status predictions for respective individuals that sourced the respective non-sequence altering change marker inputs.

32. A system, comprising:
memory storing a non-sequence altering change latent space that:
   comprises a plurality of feature embeddings that compress a plurality of non-sequence altering change marker inputs into a jointly Gaussian distribution; and
   is configured to model a non-sequence altering change clock.

### Clause Set 2—“Pixelated Non-Sequence Altering Change Marker”

1. A system, comprising:
memory storing a non-sequence altering change marker image tensor that:
   comprises respective images for respective non-sequence altering change samples,
      wherein each of the non-sequence altering change samples is represented by a plurality of modification values for a plurality of modification sites, and
      wherein each of the images uses a plurality of pixelated values to encode the plurality of modification values.

2. The system of clause 1, wherein pixelated values in the plurality of pixelated values are grayscale values.

3. The system of clause 2, wherein the pixelated values are red, green, and blue (RGB) values.

4. The system of clause 1, wherein, prior to being encoded as the plurality of pixelated values, the plurality of modification values is arranged in a matrix whose columns are sorted by chromosomal genomic location or spatial proximity in nucleus, and whose rows are sorted by genomic locations or spatial proximity of the plurality of modification sites.

5. The system of clause 4, wherein at least some elements of the matrix are empty.

6. The system of clause 1, wherein the plurality of modification values is preprocessed to remove low-variability modification values.

7. The system of clause 1, wherein the plurality of modification values is normalized using a Beta Mixture Quantile dilation (BMIQ) with an exponential-normal mixture (Enmix) signal intensity background correction.

8. The system of clause 1, wherein the images are N-bit images, where N ranges from 1 to 64.

9. A system, comprising:
memory storing a non-sequence altering change marker image tensor that:
   comprises respective images for respective non-sequence altering change samples,
      wherein each of the non-sequence altering change samples is represented by a plurality of non-sequence altering change marker values, and
      wherein each of the images uses a plurality of pixelated values to encode the plurality of non-sequence altering change marker values.

10. The system of clause 9, wherein pixelated values in the plurality of pixelated values are grayscale values.

11. The system of clause 9, the pixelated values are red, green, and blue (RGB) values.

12. The system of clause 9, wherein, prior to being encoded as the plurality of pixelated values, the plurality of non-sequence altering change marker values is arranged in a matrix whose columns are sorted by chromosomal genomic location or spatial proximity in nucleus, and whose rows are sorted by genomic locations or spatial proximity of the plurality of modification sites.

13. The system of clause 9, wherein the images are N-bit images, where N ranges from 1 to 64.

14. A system, comprising:
a variational autoencoder comprising an encoder neural network and a decoder neural network,
   the encoder neural network configured to process respective images for respective non-sequence altering change samples, and to encode the respective images in a non-sequence altering change latent space,
      wherein each of the non-sequence altering change samples is represented by a plurality of modification values for a plurality of modification sites,
      wherein each of the images uses a plurality of pixelated values to encode the plurality of modification values, and
      wherein the non-sequence altering change latent space comprises respective feature embeddings that compress the respective images into a jointly Gaussian distribution; and
   the decoder neural network configured to sample the respective feature embeddings from the non-sequence altering change latent space, and to decode the respective feature embeddings into respective outputs that are respective reconstructions of the respective images.

15. The system of clause 14, wherein pixelated values in the plurality of pixelated values are grayscale values.

16. The system of clause 14, the pixelated values are red, green, and blue (RGB) values.

17. The system of clause 14, wherein, prior to being encoded as the plurality of pixelated values, the plurality of modification values is arranged in a matrix whose columns are sorted by chromosomal genomic location or spatial proximity in nucleus, and whose rows are sorted by genomic locations of the plurality of modification sites.

18. The system of clause 14, wherein the encoder neural network is configured to generate the non-sequence altering change latent space by combining the plurality of pixelated values in a weighted, nonlinear pattern, and thereby selecting signal features from the images, and not selecting noise features from the images.

19. The system of clause 14, wherein the decoder neural network is configured to randomly sample the feature embeddings from the non-sequence altering change latent space.

20. The system of clause 14, wherein the encoder neural network is a multi-layer neural network that is interspersed with batch normalization functions and nonlinear activation functions.

21. The system of clause 14, wherein the decoder neural network is a multi-layer neural network that is interspersed with batch normalization functions and nonlinear activation functions.

22. The system of clause 14, further configured to generate the non-sequence altering change latent space by repeatedly training the variational autoencoder on the respective images over multiple epochs.

23. The system of clause 22, wherein the variational autoencoder is trained using an ADAM optimizer with a Mean Squared Error (MSE) or Binary Cross Entropy (BCE) loss function and a Kullback-Leibler divergence (KLD) loss function.

24. The system of clause 23, wherein the MSE or BSE loss function measures a reconstruction loss based on comparing the respective outputs to the respective images.

25. The system of clause 23, wherein the KLD loss function measures a distance between a distribution of the non-sequence altering change latent space and a second distribution.

26. The system of clause 25, wherein the second distribution is a Gaussian distribution, thereby causing the KLD loss function to require the non-sequence altering change latent space to approximate a normal random variable.

27. The system of clause 25, further configured to use a Monte Carlo approximation of the KLD loss function.

28. The system of clause 27, wherein the Monte Carlo approximation is distribution agnostic and calculates the distance without making any prior assumptions about any underlying distribution.

29. The system of clause 23, wherein the variational autoencoder is trained using a loss function that is defined as the MSE or BSE loss function+w*the KLD loss function, where w is a KLD weight.

30. The system of clause 29, wherein the KLD weight is set to a specific value between zero and unity.

31. The system of clause 29, wherein the KLD weight varies between zero and unity, following a cyclical sigmoid annealing scheduling to mitigate KLD vanishing.

32. The system of clause 14, wherein the feature embeddings are biology-related feature embeddings.

33. The system of clause 32, wherein the feature embeddings are age-related feature embeddings.

34. The system of clause 14, wherein a non-sequence altering change age prediction model is configured to process the feature embeddings in the non-sequence altering change latent space through a plurality of fully connected layers and generate non-sequence altering change age predictions.

35. The system of clause 34, wherein the non-sequence altering change age prediction model is interspersed with dropout functions and nonlinear activation functions.

36. The system of clause 34, wherein the non-sequence altering change age prediction model is further configured to:
select, from the non-sequence altering change latent space, a particular feature embedding of a particular image for a particular sample;
process the particular feature embedding through the plurality of fully connected layers; and
generate a non-sequence altering change age prediction for a particular individual that sourced the particular sample.

37. The system of clause 34, wherein the non-sequence altering change age prediction model is trained on the respective feature embeddings labelled with respective known non-sequence altering change ages of respective individuals that sourced the respective non-sequence altering change samples.

38. The system of clause 37, wherein the non-sequence altering change age prediction model is trained using an ADAM optimizer along with a weighted MSE or BSE loss function, where an error on each sample is weighted according to its age class weight.

39. The system of clause 1, wherein a condition status prediction model is configured to process the feature embeddings in the non-sequence altering change latent space and generate condition status predictions.

40. The system of clause 39, wherein the condition status prediction model is a binary of multi-class classification model.

41. The system of clause 39, wherein the condition status prediction model is further configured to:
select, from the non-sequence altering change latent space, a particular feature embedding of a particular image for a particular sample;
process the particular feature embedding; and
generate a condition status prediction for a particular individual that sourced the particular sample.

42. The system of clause 39, wherein the condition status prediction model is trained on the respective feature embeddings labelled with respective known condition statuses of respective individuals that sourced the respective non-sequence altering change samples.

43. The system of clause 42, wherein the condition status prediction model is trained using an ADAM optimizer along with a weighted MSE or BSE loss function, where an error on each sample is weighted according to its age class weight.

44. A system, comprising:
a variational autoencoder comprising an encoder neural network and a decoder neural network,
   the encoder neural network configured to process respective images for respective non-sequence altering change samples, and to encode the respective images in a non-sequence altering change latent space,
      wherein each of the non-sequence altering change samples is represented by a plurality of modification values for a plurality of modification sites,
      wherein each of the images uses a plurality of pixelated values to encode the plurality of modification values, and
      wherein the non-sequence altering change latent space comprises respective feature embeddings that compress the respective images into a jointly Gaussian distribution; and
   the decoder neural network configured to sample the respective feature embeddings from the non-sequence altering change latent space, and to decode the respective feature embeddings into respective outputs that are respective reconstructions of the respective images; and
a non-sequence altering change age prediction model configured to process the respective feature embeddings and generate respective non-sequence altering change age predictions for respective individuals that sourced the respective non-sequence altering change samples.

45. A system, comprising:
a variational autoencoder comprising an encoder neural network and a decoder neural network,
   the encoder neural network configured to process respective images for respective non-sequence altering change samples, and to encode the respective images in a non-sequence altering change latent space,
      wherein each of the non-sequence altering change samples is represented by a plurality of modification values for a plurality of modification sites,
      wherein each of the images uses a plurality of pixelated values to encode the plurality of modification values, and
      wherein the non-sequence altering change latent space comprises respective feature embeddings that compress the respective images into a jointly Gaussian distribution; and
   the decoder neural network configured to sample the respective feature embeddings from the non-sequence altering change latent space, and to decode the respective feature embeddings into respective outputs that are respective reconstructions of the respective images; and
a condition status prediction model configured to process the respective feature embeddings and generate respective condition status predictions for respective individuals that sourced the respective non-sequence altering change samples.
   Clause Set 3—Detecting Rheumatoid Arthritis with Marker Genes

1. A computer-implemented computer-implemented method of forming marker genes for predicting the likelihood of a patient contracting rheumatoid arthritis, the computer-implemented method comprising:
obtaining a first set of unique blood product samples from multiple subjects who have been diagnosed with rheumatoid arthritis;
obtaining a second set set of unique blood product samples from a control group of healthy subjects who are free of rheumatoid arthritis;
quantifying the DNA of each sample using fluorescence-based nucleic acid analysis;
performing bisulfite conversion a selected nanogram weight of each sample;
performing PCR to amplify the DNA to maximize the methylation signal;
maximizing DNA methylation on all samples using an assay kit that preserves methylated sites while non-methylated locations are replaced;
hybridizing the DNA fragments in a methylation bead-chip array, in which the DNA fragments bind to matching silica beads; and
using a DL classifier algorithm, classifying the DNA fragments to select CpG marker genes associated with CpG islands indicative of the presence of rheumatoid arthritis.

2. The computer-implemented method of clause 1, further including flanking primers for amplifying the region of DNA containing CpG for identifying rheumatoid arthritis sites.

3. The computer-implemented method clause 1, wherein the fluorescence-based nucleic acid analysis is performed using dsDNA (double strand) fluorometry.

4. The computer-implemented method of clause 1, wherein the classifier algorithm classifies genomic blood product data as positive for rheumatoid arthritis or negative for rheumatoid arthritis based on the presences of selected marker genes in the blood product sample.

5. The computer-implemented method of clause 1, further including assaying a blood product sample associated with a new patient to identify if any selected marker genes are present.

6. The computer-implemented method of clause 1, wherein a deep learning model is trained using a deep model architecture that includes a variational autoencoder.

7. The computer-implemented method of clause 1, wherein selected marker genes are formed for predicting diseases other than or in addition to rheumatoid arthritis in a patient.

8. The computer-implemented method of clause 1, wherein wherein the DNA segments are assayed to detect the RCAN3 gene for distinguishing rheumatoid patients from controls patents.

9. The computer-implemented method of clause 1, wherein wherein the DNA segments are assayed to detect the HDAC3 gene for distinguishing rheumatoid patients from healthy control patents.

10. The computer-implemented method of clause 1, wherein wherein the DNA segments are assayed to detect the SIPA1 gene for distinguishing rheumatoid patients from healthy control patients.

11. A computer-implemented method of assaying a patient's blood products to identify a marker gene selected from a group of marker genes consisting of RCAN3, HDAC4, and SIPA1 for distinguishing rheumatoid patients from healthy control patients.

12. A RCAN3 marker gene for distinguishing rheumatoid patents from healthy control patients.

13. A HDAC4 marker gene for distinguishing rheumatoid patents from healthy control patients.

14. A SIPA1 marker gene for distinguishing rheumatoid patients from healthy control patients.

What is claimed is:

What is claimed is:

1. A system, comprising:
a variational autoencoder comprising an encoder neural network and a decoder neural network,
the encoder neural network configured to process a plurality of non-sequence altering change marker inputs, and to encode the plurality of non-sequence altering change marker inputs in a non-sequence altering change latent space,
wherein the non-sequence altering change latent space comprises respective feature embeddings that compress respective non-sequence altering change marker inputs in the plurality of non-sequence altering change marker inputs into a jointly Gaussian distribution; and
the decoder neural network configured to sample the respective feature embeddings from the non-sequence altering change latent space, and to decode the respective feature embeddings into respective outputs that are respective reconstructions of the respective non-sequence altering change marker inputs.

2. The system of claim 1, wherein the variational autoencoder is trained using an ADAM optimizer with a Mean Squared Error (MSE) or Binary Cross Entropy (BCE) loss function and a Kullback-Leibler divergence (KLD) loss function.

3. A computer-implemented computer-implemented method of forming marker genes for predicting the likelihood of a patient contracting rheumatoid arthritis, the computer-implemented method comprising:
obtaining a first set of unique blood product samples from multiple subjects who have been diagnosed with rheumatoid arthritis;
obtaining a second set set of unique blood product samples from a control group of healthy subjects who are free of rheumatoid arthritis;
quantifying the DNA of each sample using fluorescence-based nucleic acid analysis;
performing bisulfite conversion a selected nanogram weight of each sample;
performing PCR to amplify the DNA to maximize the methylation signal;
maximizing DNA methylation on all samples using an assay kit that preserves methylated sites while non-methylated locations are replaced;
hybridizing the DNA fragments in a methylation bead-chip array, in which the DNA fragments bind to matching silica beads; and
using a DL classifier algorithm, classifying the DNA fragments to select CpG marker genes associated with CpG islands indicative of the presence of rheumatoid arthritis.

4. The computer-implemented method of claim 1, further including flanking primers for amplifying the region of DNA containing CpG for identifying rheumatoid arthritis sites.

5. The computer-implemented method claim 1, wherein the fluorescence-based nucleic acid analysis is performed using dsDNA (double strand) fluorometry.

6. The computer-implemented method of claim 1, wherein the classifier algorithm classifies genomic blood product data as positive for rheumatoid arthritis or negative for rheumatoid arthritis based on the presences of selected marker genes in the blood product sample.

7. The computer-implemented method of claim 1, further including assaying a blood product sample associated with a new patient to identify if any selected marker genes are present.

8. The computer-implemented method of claim 1, wherein a deep learning model is trained using a deep model architecture that includes a variational autoencoder.

9. The computer-implemented method of claim 1, wherein selected marker genes are formed for predicting diseases other than or in addition to rheumatoid arthritis in a patient.

10. The computer-implemented method of claim 1, wherein wherein the DNA segments are assayed to detect the RCAN3 gene for distinguishing rheumatoid patients from controls patents.

11. The computer-implemented method of claim 1, wherein wherein the DNA segments are assayed to detect the HDAC3 gene for distinguishing rheumatoid patients from healthy control patents.

12. The computer-implemented method of claim 1, wherein wherein the DNA segments are assayed to detect the SIPA1 gene for distinguishing rheumatoid patients from healthy control patients.

13. A computer-implemented method of assaying a patient's blood products to identify a marker gene selected from a group of marker genes consisting of RCAN3, HDAC4, and SIPA1 for distinguishing rheumatoid patients from healthy control patients.

14. A RCAN3 marker gene for distinguishing rheumatoid patents from healthy control patients.

15. A HDAC4 marker gene for distinguishing rheumatoid patents from healthy control patients.

16. A SIPA1 marker gene for distinguishing rheumatoid patients from healthy control patients.
